(19) United States
(12) Patent Application Publication
(10) Pub. No.: US 2006/0204974 A1
(43) Pub. Date: Sep. 14, 2006

(54) Methods and agents for regulating angiotensin activity

(75) Inventor: Kathryn Sandberg, Silver Spring, MD (US)

Correspondence Address: FISH & NEAVE IP GROUP;ROPES & GRAY LLP, ONE INTERNATIONAL PLACE, BOSTON, MA, 02110-2624, US

(73) Assignee: Georgetown University, Washington, DC (US)

(21) Appl. No.: 11/235,785

(22) Filed: Sep. 27, 2005

Related U.S. Application Data
(63) Continuation-in-part of application No. 10/951,091, filed on Sep. 27, 2004.
(60) Provisional application No. 60/506,191, filed on Sep. 26, 2003.
(60) Provisional application No. 60/613,735, filed on Sep. 27, 2004.

(51) Int. Cl. A61K 48/00; C12Q 1/68; C07H 21/02

(52) U.S. Cl. 435/6; 514/44; 435/320.1; 536/23.1

(57) ABSTRACT

In certain aspects, the present invention relates to methods and preparations for treating angiotensin II-mediated diseases, and in particular, methods of screening for agents that modulate post-transcriptional regulation of angiotensin II receptors and the use of such agents.

### RELATED APPLICATION

This application is a continuation-in-part of U.S. patent application Ser. No. 10/951,091 Methods and Agents for Regulating Angiotensin Activity filed Sep. 27, 2004, which claims the benefit of the filing date of U.S. Provisional application No. 60/506,191, entitled Methods and Agents for Regulating Angiotensin Activity filed Sep. 26, 2003; it also claims the benefit of U.S. Provisional Application No. 60/613,735, entitled Methods and Agents for Regulating Angiotensin Activity filed Sep. 27, 2004. The entire teachings and specification of each of the referenced applications are incorporated herein by reference.

### FUNDING

This invention was made with government support under Grant #RO1 HL57502 awarded by the National Institutes of Health. The United States government has certain rights in the invention.

### BACKGROUND OF THE INVENTION

The renin-angiotensin system (“RAS”) plays an integral role in maintaining vascular tone, optimal salt and water homeostasis, and cardiac function in humans. Angiotensin II is a peptide hormone produced mainly in the blood during the cleavage of angiotensin I by angiotensin-converting enzyme (“ACE”) which is localized on the endothelium of blood vessels of lung, kidney, and many other organs. Angiotensin II interacts with specific receptors on the surface of the target cells and is the effector of the RAS. Two receptor subtypes have been identified: AT1 receptors (“AT1Rs”) and AT2 receptors (“AT2Rs”). The type-1 angiotensin II receptor (“AT1R”) is a G protein-coupled receptor and mediates most of the biological actions of angiotensin II through activation of a phosphatidylinositol-calcium second messenger system (Murphy et al., Nature 1991; 351: 233-236). In recent years, it has been recognized that pathologic consequences may result from overactivity of angiotensin II-mediated signaling pathways. Examples of angiotensin II-mediated diseases include renal artery stenosis, hypertension, diabetic and nondiabetic nephropathies, left ventricular hypertrophy, coronary atherosclerosis, myocardial infarction, and congestive heart failure (Brewster et al., Am. J. Med. Sci. 2003; 326: 15-24).

Recently, considerable efforts have been made to identify substances that bind selectively to the AT1R. These active compounds are called angiotensin II antagonists or angiotensin II receptor blockers (“ARBs”). As a consequence of the inhibition of the ATLR, these antagonists can, for example, be employed as anti-hypertensives or for treating congestive heart failure (Ramahi, Postgrad. Med 2001; 109:115-122). The recently developed class of ARBs appear to be as effective as ACE inhibitors in delaying the progression of renal injury in animal models of diabetes (Barnett, Blood Press 2001; 10 Suppl 1:21-26). They act by selectively blocking the binding of angiotensin II to AT1R and may therefore offer a more complete blockade of RAS than ACE inhibitors, which inhibit the conversion of angiotensin I to angiotensin II. With the AT1R blocked, angiotensin II is available to activate the type-2 angiotensin receptor (“AT2R”), which mediates several potentially beneficial effects in the cardiovascular system, including vasodilation, antiproliferation, and apoptosis (Siragy, Am J Cardiol. 1999; 84:3S-8S).

Several polymorphisms in the human AGTR1 gene have been discovered, some of which have been reported to be associated with hypertension. For example, Bonnardeaux et al. (Hypertension 1994; 24:63-69) identified an adenine or cytosine polymorphism (A1166C) located in the 3-prime untranslated region of the AT1R gene. This variant was present at a significantly elevated frequency in 206 Caucasian patients with essential hypertension. Wang et al. (Clin. Genet. 1997; 51:31-34) did a case-control study of the A1166C variant in a well-characterized group of 108 Caucasian hypertensive subjects with a strong family history (two affected parents) and early onset disease. The frequency of the A1166C allele in this subject group was 0.40 in hypertensives compared to 0.29 in normotensives. Further characterization of the A1629C polymorphism has shown it is significantly more frequent in women who develop pregnancy-induced hypertension as compared to healthy controls (Nalogowska-Glosnicka et al., Med Sci. Monit. 2000; 6:523-529). These data further support the notion that AT1R is an important target for the control of angiotensin II-dependent hypertension.

Angiotensin II has also been implicated in the development of cardiac hypertrophy, because ACE inhibitors and ARBs prevent or regress ventricular hypertrophy in animal models and in humans. Herzig et al. studied AT1R promoter activity during cardiac hypertrophy, and discovered that AT1R expression is enhanced 160% in hypertrophied myocardium compared to normal myocardium (Proc. Natl. Acad. Sci. U.S.A 1997; 94:7543-7548).

The AT1R gene is located on chromosome 3q21-q25 and contains one exon that encodes a 359 amino acid protein. Two human AT1R subtypes have been identified, and recent evidence has indicated there may be as many four AT1R splice variants that are expressed in humans (Martin et al., Mol. Endocrinol. 2001; 15:281-293). Reference sequences for the AT1R gene (Genaissance Reference No. 2506603; SEQ ID NO:1), coding sequence (GenBank Accession No:NM000685.2).

In all angiotensin receptors thus far cloned, the coding region of the receptor is contained within one exon. Splicing of AT receptors occurs exclusively within the 5′ leader sequence (5′LS) Elton and Martin (Trends Endocrinol. Metabl. 2003; 14: 66-71). Genomic analysis of mouse, rat, and human angiotensin receptors, suggests that alternative splicing within the 5′ leader sequence (“5′LS”) is a common event in mammalian AT1Rs and AT2Rs. The rat AT1R gene has 3 exons, and two distinct alternatively spliced transcripts are expressed in rat tissues. In the rat, there are two subtypes of the AT1R (AT1aR and AT1bR). The AT1aR is widely distributed in Ang II target tissues including the aorta, heart, spleen, liver, brain and kidney. In contrast, the distribution of the AT1bR is more limited with greatest expression occurring in the pituitary and adrenal (Llorens-Cortes et. al., Hypertension 1994; 24: 538-548). The 5′LS of both the AT1aR and the AT1bR are encoded by exon 1 (E1), exon 2 (E2) and 52 and 47 nucleotides, respectively, of exon 3 (E3). The rest of E3 in both receptors harbors the entire open reading frame plus the 3′ untranslated region. Human AT1R has also been shown to have 4 exons, and 4 distinct alternatively spliced transcripts expressed in human tissues in varying abundance. Curnow (Clin. Exp. Pharmacol. Physiol. Suppl. 1996; 3:S67-73) and Curnow et al. (Mol. Endocrinol. 1995, 9:1250-62) showed that the human exon 2-containing transcript had a markedly lower translation of the downstream open reading frame compared to the transcripts lacking exon 2 and suggested that the inhibitory effect of exon 2 on translation was due to a minicistron commencing with an ATG in an optimal context for translation initiation. Warnecke et al. (J. Mol. Med. 1999, 77: 718-27) analyzed the alternatively spliced AT1R transcripts in endomyocardial biopsies and found that in failing hearts, the percentage of exon-containing transcripts (out of the total AT1R mRNA) was significantly reduced in atria and in the left ventricle. There is a need to develop agents and methods that can regulate the translation and expression of AT1R splicing variants.

### SUMMARY OF THE INVENTION

This invention relates to methods and preparations for treating angiotensin II-mediated diseases, and in particular, methods of screening for agents that modulate post-transcriptional regulation of angiotensin II receptors and the use of such agents. As described herein, it has been determined that alternative splicing of the AT1R gene plays an important role in regulating its translation and expression of AT1R proteins. For example, an alternatively spliced transcript lacking exon 2 shows enhanced translation and results in higher density of AT1R proteins expressed in the respective cell membrane, relative to the alternatively spliced transcript including exon2. Results showed higher AT1aR protein levels from E1,3, compared to E1,2,3 transcripts, suggesting that exon2 reduces functional AT1R expression by inhibiting translation. Also described herein is the finding that the angiogension II—mediated signaling can be modulated by altering cis or trans elements that control translational efficiency of splicing variants of an AT1R.

Deletion of 10 nucleotides in exon2 increased translation of the mutated E1,2,3 transcript to levels indistinguishable from E1,3, supporting the conclusion that this loop region of a predicted hairpin contributes to the inhibitory RNA cis element within exon2.

The present invention is directed to methods and preparations relating to angiotensin II-mediated diseases. Specifically, the invention relates to methods of screening for agents that regulate angiotensin receptor genes at the post-transcriptional level, pharmaceutical preparations comprising such agents, and uses of such agents in treating angiotensin II-mediated diseases.

In one aspect, the present invention provides methods of screening for agents that regulate alternative splicing of AT1R gene. In a specific embodiment, the screening method identifies agents that increase the ratio of exon 2-containing transcript (“E2” or “E 1,2,3 ”) to the transcript without exon 2 (“ΔE2” or “E1,3”), and in a further embodiment, the agents increase the ratio of E2/ΔE2 without changing the total amount of AT1R mRNA. In another embodiment, the screening method identifies agents that promote alternative splicing resulting in E2 or inhibit alternative splicing resulting in ΔE2 or both, and in a specific embodiment, the agents increase the ratio of E2/ΔE2 without changing the total amount of AT1R mRNA.

In certain aspects, the present invention provides methods of screening for an agent that reduces angiotensin II-mediated signaling in a cell, comprising identifying an agent that decreases translational efficiency of one or more splicing variant of an AT1R gene (e.g., human AT1R gene). In a specific embodiment, siRNAs are candidate agents to be screened. For example, siRNAs targeting the junction between two different exons are the candidate agents to be screened.

In a specific embodiment, siRNAs are candidate agents to be screened. In particular embodiments, siRNAs targeting the junction between exons 1 and 3, which is unique to ΔE2, are the candidate agents to be screened.

In a specific embodiment, small molecules such as small molecules are generated by combinatorial synthesis, are candidate agents to be screened.

Yet another aspect of the screening method identifies agents that selectively inhibit translation of the ΔE2 transcript. In particular embodiments, the agents are antisense polynucleotides that specifically attenuate the expression of ΔE2 transcript.

Another aspect of the present invention is directed to an expression vector comprising a nucleic acid encoding an antisense molecule, an siRNA molecule, or a peptide that regulates AT1R post-transcriptionally.

In certain aspects, the present invention provides expression vectors that comprise a nucleic acid encoding an siRNA or a hairpin RNA that specifically targets a region of an AT1R gene (e.g., human AT1R). In certain cases, the region is either an exon of the AT1R gene or a juncture between two different exons of the AT1R gene. Optionally, the siRNA or the hairpin RNA specifically targets a certain splice variant of the AT1R gene.

In a specific aspect, the present invention provides an isolated siRNA or a hairpin RNA that specifically targets a splice variant of an AT1R gene.

In certain aspects, the present invention provides expression vectors that comprise a nucleic acid encoding an antisense sequence that attenuates the expression of a splicing variant of an AT1R gene.

In a specific aspect, the present invention provides an isolated antisense nucleotide sequence that specifically targets a splice variant of an AT1R gene.

Another aspect of the invention is directed to transgenic animals. The subject transgenic animal may have attenuated expression of native AT1R gene due to a transgene encoding an antisense molecule, or an siRNA molecule of the present invention. Alternatively, the transgenic animal may express AT1R from a transgene encoding the E2 or ΔE2 transcript. In a particular embodiment, the native AT1R gene is replaced through homologous recombination by a transgene encoding the E2 or ΔE2 transcript.

Another aspect of the invention relates to the discovery that specific RNA-binding proteins (RNABPs) regulate the translation of AT1R mRNA. In one embodiment, the subject screening method identifies agents that modulate the activities of the RNABPs, and thereby regulate the translation of AT1R transcripts. In a further embodiment, the RNABPs that inhibit translation of ΔE2 serve as targets to screen for agents that facilitate the inhibitory effect of these RNABPs on translation. The agents that target the RNABPs can be small molecules, proteins, antibodies, nucleic acids encoding polypeptides that modulate the RNABPs, or generally agents that promote the function of these RNABPs.

A further aspect of the invention is directed to gene therapy utilizing a desired agent of the present invention. Examples without limitation include an RNAi construct of the present invention.

In certain embodiments, the candidate agents regulating AT1R mRNA translation and expression do not affect translation and/or expression of the AT2R gene. In other embodiments, the candidate agents regulating AT1R mRNA translation and expression may promote expression of the AT2R gene post-transcriptionally, (enhancing translation or facilitating expression or both).

Another aspect of the present invention provides pharmaceutical preparations comprising the agents having the recited property, for example, agents capable of increasing the ratio of E2/ΔE2 without changing the total amount of AT1R mRNA.

In a particular embodiment, the present invention provides a vector suitable for gene therapy comprising siRNAs having recited properties.

In one embodiment, the present invention provides a vector comprising nucleic acids encoding polypeptides that modulate the RNABP's activities.

Another aspect of the present invention relates to the use of the agents as discussed above to treat angiotensin II-mediated diseases. In one embodiment, the subject method comprises administering to a patient in need thereof a pharmaceutical preparation comprising such agents. In certain embodiments, the patient suffers from a condition such as hypertension or cardiac hypertrophy.

Still another aspect of the present invention provides a method of conducting a pharmaceutical business comprising:

a). identifying an agent that increases the ratio of E2/ΔE2, promotes the translation of E2, or inhibits the translation of ΔE2;

b). conducting therapeutic profiling of the agent identified in step (a) for efficacy and toxicity in animals; and

c). formulating a pharmaceutical preparation including one or more agents identified in step (b) as having an acceptable therapeutic profile.

Preferably, the method of conducting a pharmaceutical business further includes establishing a distribution system for distributing the pharmaceutical preparation for sale, and (optionally) establishing a sales group for marketing the pharmaceutical preparation.

Yet still another aspect of the present invention provides a method of conducting a pharmaceutical business comprising:

a). identifying an agent that increases the ratio of E2/ΔE2, promotes the translation of E2, or inhibits the translation of ΔE2;

b). (optionally) conducting therapeutic profiling of the agent identified in step (a) for efficacy and toxicity in animals; and

c) licensing, to a third party, the rights for further development of the agent.

### DESCRIPTION OF DRAWINGS

FIG. 1 is a schematic view of angiotensin II-mediated signaling leading to changes in blood pressure. FIG. 2 is a schematic view of two distinct rat angiotensin type 1a receptor (AT1aR): ΔE2 vs. E2. CR represents the coding region.

FIG. 3 shows that E2 is less efficiently translated in vitro than ΔE2. Lane 1: negative control; Lane 2: positive control; Lane 3: E2; Lane 4: ΔE2 for the in vitro translation assays.

FIGS. 4A-4B show that AT1aR density is higher in rat aortic smooth muscle cells (A10) transfected with ΔE2 versus E2 plasmid DNA. FIG. 4A: AT, specific binding and FIG. 4B: AT, B max. FIG. 5 shows that E2m (AT1aR E2 mutant with all four AUGS in the 5′ LS disrupted) is more efficiently translated in vitro than E2.

FIGS. 6A & 6B show that AT1aR density is significantly higher in A10 cells transfected with E2m compared to E2 plasmid DNA.

FIG. 6C shows that Angiotensin II-induced IP production is significantly higher in E2m plasmid DNA transfected cells than that in E2 plasmid DNA transfected cells.

FIG. 7 shows the presence of E2 (E1,2,3) and ΔE2 (E1,3) splicing variants in different tissues. RASMC: rat aortic smooth muscle cells.

FIG. 8 shows the ratio of renal cortex ΔE2/E2 in different strains of rats.

FIG. 9A presents results that show that two splicing variants of AT1aR exist in rat renal cortex as shown by real-time PCR using primers specific to E2 and ΔE2 variants with total RNA isolated from the renal cortex of Sprague Dawley (SD) rats.

FIG. 9B shows that the ratio of ΔE2 to total AT1aR mRNA is higher in normotensive DS rats than that in SD or Fischer 344/BN rats.

FIG. 10 shows that the ratio of ΔE2 to total AT1aR mRNA is higher in hypertensive DS rats maintained on HS diet than in hypertensive DS rats maintained on NS diet.

FIG. 11 shows representative 21-nucleotide siRNAs. T: AT1R ΔE2 target sequence. S: sense strand. AS: antisense strand. (SEQ ID NOS: 15-53)

FIG. 12 shows representative 22-, 23-, 24-, 25-nucleotide siRNAs. T: AT1R ΔE2 target sequence. S: sense strand. AS: antisense strand. (SEQ ID NOS: 54-77)

FIG. 13. Schematic representation of AT1aR gene and the two splice variants. The AT1aR gene is encoded by E1, E2 and 52 nucleotides of E3. Two splice variants could exist (E1,3 and E1,2,3), which differ only in the lengths of their 5′LS since E3 harbors the entire open reading frame for the AT1aR plus the 3′ untranslated region.

FIG. 14A-14D show AT1aR density in CHO cells stably transfected with E1,3 and E1,2,3. FIG. 14A: Saturation curves of 1251-[Sar1,Ile8]Ang II binding to membranes from CHO cells stably expressing E1,3 and E1,2,3 transcripts are shown. FIG. 14B: Scatchard plot of binding data. FIG. 14C: AT1R Bmax values obtained from Scatchard analysis of the saturation curves for E1,3 and E1,2,3. FIG. 14D: AT1R Bmax values normalized to E1,3 or E1,2,3 mRNA levels. The data are averaged from 3 experiments on 3 individual clones per transcript; each saturation binding curve is performed in triplicate.

FIG. 15A-15B show Ang II-induced inositol phosphate production in CHO cells stably transfected with E1,3 and E1,2,3 transcripts. FIG. 15A: Shown are the levels of Ang II-stimulated IP accumulation after a 20 min incubation with 100 nM Ang II, defined as total IP accumulated in the presence of Ang II minus the levels accumulated in the presence of vehicle. The data are expressed in cpm. FIG. 15B: Shown is the total IP accumulated (defined above) normalized to AT1R Bmax. The data are averaged from 3 experiments on 3 individual clones per transcript; each IP assay is performed in quadruplicate.

FIG. 16A-16B. RNA stability of the E1,3 and E1,2,3 transcripts stably expressed in CHO cells. FIG. 16A: CHO cells stably expressing E1,3 and E1,2,3 were treated with actinomycin D before E1,3 and E1,2,3 mRNA were determined by RNase protection assay as a function of time. The data are averaged from mRNA turnover assays performed in triplicate on 3 separate clones for each transcript; each assay is performed in triplicate. FIG. 16B: The levels of E1,3 and E1,2,3 mRNA in stably transfected CHO cells are shown. The data are averaged from 6 real-time PCR assays performed in duplicate.

FIG. 17A. In vitro translation of E1,3 and E1,2,3 capped RNA. Upper panel, Shown are phosphorimages of in vitro translated proteins run on an SDS gel. Lane 1, negative control (no RNA template); Lane 2, positive control (BMV, bovine mosaic virus); Lane 3, E1,2,3; Lane 4, Mut E1,2,3, Lane 5, E1,3. Left panel, Shown are the amounts of protein translated for the E1,3, E1,2,3 and Mut E1,2,3 transcripts. The data are expressed in arbitrary units (AU). The data are averaged from 3 experiments each performed in triplicate. FIG. 17B, Hairpin in E2 predicted by the Zuker algorithm and the predicted hairpin containing the 10 nucleotide deletion.

FIG. 18. Northern blot of AT1aR exons in rat adrenal cortex. Shown are three Northern blots of adrenal cortex total RNA hybridized with E1, E2 or E3 [32P]-dCTP probes. Each lane represents adrenal cortex total RNA from 2 animals.

FIG. 19A-19D. Correlation between AT1R Bmax and Splice variants. Shown is the relationship between AT1R Bmax and the levels of E1,2,3 FIG. 19A, E1,3 FIG. 19B and total AT1aR (E1,2,3+E1,3) FIG. 19C mRNA levels in heart (H), spleen (S), liver (L), and preglomerular (PG) tissues. FIG. 19D: Shown is the relationship between AT1R Bmax and the percentage of E1,3 in the total AT1aR mRNA population in these same tissues. The AT1R Bmax data are averaged from Scatchard plots from 3 radioligand binding curves per tissue. The E1,3, E1,2,3 and total AT1aR mRNA levels are determined by real-time PCR with an n of 6 per tissue; each real-time PCR reaction was performed in duplicate.

FIG. 20A-20B. Design of siRNA duplexes targeting AT1a receptor splice variants. There are two AT1a receptor splice variants. The first (E1,2,3) contains all three exons while the second contains only exons 1 and 3 (E1,3). B) S1E1,3 siRNA was designed to target the junction between exons 1 and 3 and thus was specific for the E1,3 splice variant. S2E3 siRNA was designed to target exon 3 and thus was directed against both E1,3 and E1,2,3 mRNA. Additionally two control siRNAs (which did not match any known rat or Chinese hamster sequences) were selected for use as controls. (SEQ ID NOS: 78-85)

FIG. 21A-21B. RNAi of AT1a receptor splice variants stably expressed in Chinese Hamster Ovary (CHO) cells. FIG. 21A: Treatment with both S1E1,3 and S2E3 siRNA significantly reduced AT1 receptor binding compared with the control in CHO cells stably expressing the E1,3 AT1a receptor splice variant. FIG. 21B: Treatment with S2E3siRNA, but not S1E1,3 siRNA, induced a significant reduction in binding compared with the control in CHO cells stably expressing the E1,2,3 splice variant. Data are expressed as the mean±SEM. ***, p<0.001.

FIG. 22: Predominance of E1,2,3 mRNA in RASMC. Quantitative real-time PCR of AT1a receptor transcripts demonstrated that E1,2,3 mRNA makes up 75.9±1.9% (n=4) of the total AT1a receptor mRNA in RASMC. This ratio was unaffected by treatment of cells with control siRNA. Data are expressed as the mean±SEM.

FIG. 23A-23B: Splice variant-specific RNAi of AT1a receptor transcripts in RASMC. 48 h following siRNA treatment, cells treated with S1E1,3 siRNA showed marked reductions in E1,3 mRNA but not E1,2,3 mRNA. Treatment with S2E3 siRNA caused significant reductions in levels of both E1,3 and E1,2,3 mRNA. Data are expressed as the mean±SEM. *, p<0.05; **, p<0.01.

FIG. 24A-24B: Knockdown of E1,3 mRNA causes a disproportionately large reduction in AT1 receptor binding. FIG. 24A: Although S1E1,3 siRNA treatment induced a precipitous reduction in E1,3 mRNA since E1,3 mRNA makes up only 24% of AT1a receptor mRNA, the effect on total AT1a receptor mRNA levels was not detectable. In contrast, S2E3siRNA (which targets both splice variants) induced a marked knockdown in total AT1a receptor mRNA. FIG. 24B: In contrast to its lack of effect on total AT1a receptor mRNA, S1E1,3 siRNA treatment induced a large and significant reduction in AT1 receptor binding. Data are mean±SEM. *, p<0.05; ***, p<0.001.

FIG. 25. Schematic representation of the rat AT1aR gene and the mutant constructs

FIG. 26A-26F: Comparison of WT and QM AT1R binding in transiently transfected A10 cells. FIG. 26A: Saturation isotherms of 125I-[Sar1,Ile8]Ang II binding to membranes from A10 cells transiently transfected with WT and QM plasmid DNA, using a computerized nonlinear regression analysis program, PRISM. Shown are representative data from 3 transfection experiments each performed in triplicate. FIG. 26B: Scatchard plot of the saturation isotherm data. FIG. 26C: AT1R Bmax values obtained from Scatchard analysis of the saturation curves for WT and QM expressing cells. FIG. 26D: Beta galactosidase (β-Gal) activity in A10 cells co-transfected with β-Gal and WT or β-Gal and QM plasmid DNA. FIG. 26E: AT1R mRNA levels determined by RNase protection assay in A10 cells transiently expressing WT and QM normalized to β-actin. The data are averaged from 3 transfection experiments performed in triplicate. FIG. 26F: AT1R Bmax values normalized to AT1R mRNA levels in WT and QM expressing cells.

FIG. 27A-27F. Comparison of WT and QM AT1R binding in stably transfected CHO cells. FIG. 27A: Saturation isotherms of 125I-[Sar1,Ile8]Ang II binding to membranes from CHO cells stably transfected with WT or QM plasmid DNA, using a computerized nonlinear regression analysis program, PRISM. Shown are representative data from 3 binding experiments each performed in triplicate on each clone. FIG. 27B: Scatchard plot of the saturation isotherm data in A. FIG. 27C: AT1R Bmax values obtained from Scatchard analysis from WT-1, WT-2, and WT-3 and from QM-1, QM-2 and QM-3. The data are averaged from 3 saturation isotherms performed in triplicate on each clone. FIG. 27D: AT1R Bmax values in WT and QM averaged from WT-1, WT-2 and WT-3 and from QM-1, QM-2 and QM-3. FIG. 27E: AT1R mRNA levels determined by real-time PCR and averaged from WT-1, WT-2 and WT-3 and from QM-1, QM-2 and QM-3. The data for each clone are averaged from three experiments performed in triplicate. FIG. 27F: AT1R Bmax values for all six clones normalized to their respective AT1R mRNA levels.

FIG. 28A-28D: Comparison of WT and QM AT1R signaling. FIG. 28A: Dose-response curves for Ang II-stimulated IP accumulation in CHO cells transiently transfected with QM and WT (Inset). The data are expressed as the amount of Ang II-stimulated IP accumulation defined as the total levels of IP accumulated in the presence of Arig II minus basal levels over a 20 min period as a function of Ang II concentration. The data are representative of 2 experiments each performed in triplicate. FIG. 28B: Ang II-stimulated IP accumulation in WT-1, WT-2, WT-3, QM-1, QM-2, and QM-3. Shown are the levels of Ang II-stimulated IP accumulation after a 20 min incubation with 100 nM Ang II. The data are averaged from 2 experiments on each clone performed in quadruplicate. FIG. 28C: Ang II-stimulated IP production averaged from WT-1, WT-2 and WT-3 and from QM-1, QM-2 and QM-3. FIG. 28D: Ang II-stimulated IP production normalized to their respective AT1R Bmax levels and averaged from WT-1, WT-2 and WT-3 and from QM-1, QM-2 and QM-3.

FIG. 29A-29B: Comparison of WT and QM AT1R translation. FIG. 29A: Polysome profile of WT (open squares) and QM (closed squares) expressed in A10 cells. Shown are the amounts of WT and QM mRNA in each fraction expressed as a percentage of the total AT1R mRNA recovered from all six fractions. *p<0.005, WT vs. QM; n=3. FIG. 29B: IVT of WT and QM capped RNA. Shown is the amount of protein translated for WT versus the QM transcript expressed in arbitrary units (AU). The data are averaged from 3 experiments each performed in triplicate. Inset, Shown are phosphorimages of IVT proteins run on an SDS gel. Lane 1, negative control (no RNA); Lane 2, bovine mosaic virus positive control; Lane 3, WT, and Lane 4, QM.

FIG. 30A-30B. Effect of disrupting both AUGs in exon 1 versus both AUGs in exon 2 on AT1R density and IVT. FIG. 30A: AT1R Bmax values obtained from Scatchard analysis of the saturation curves for A10 cells expressing WT, DM1, DM2, E1,3 and QM. The data are averaged from 3 binding experiments each performed in triplicate. FIG. 30B: Levels of AT1R protein translated from WT, DM1, DM2 and QM transcripts and expressed in arbitrary units (AU). The data are averaged from 3 experiments each performed in triplicate.

FIG. 31A-31B: Effect of disrupting individual upstream AUGs in exon 2 on AT1R density and IVT. FIG. 31A: AT1R Bmax values obtained from Scatchard analysis of the saturation curves for A10 cells expressing WT, M1, M2, and QM. The data are averaged from 3 experiments each performed in triplicate. FIG. 31B: Levels of AT1R protein translated from WT, M1, M2, and QM transcripts and expressed in arbitrary units (AU). The data are averaged from 3 experiments each performed in triplicate.

FIG. 32 shows human proteins from HEK 293 cells (human embryonic kidney cell line that expresses the human AT1R endogenously) bind to 32P-labeled human AT1R exon 2 RNA in a dose dependent manner.

FIG. 33 shows RNA binding protein complex in FIG. 32 is specific-unlabeled exon 2 RNA competes but other RNAs (poly A RNA and Transfer RNA) do not compete, indicating that the exon 2 in the human AT1R mRNA specifically binds to proteins in human cells.

FIG. 34 shows the effect of transfecting HEK293 cells with siRNAs targeted towards exon 4 (present in all hAT1R mRNA transcripts), exon 2 containing transcripts (E1,2,4 & E1,2,4) and the E1,3,4 transcript. The data are expressed as a percentage of the total E1,2,3,4 and E1,2,4 mRNA in the HEK293 cells normalized to the mRNA levels present in the non-silencing siRNA transfected cells. These data indicate that siRNA targeted towards exon 4 effectively knocks down all hAT1R transcript mRNA and that the siRNA targeted towards exon 2, effectively knocks down all variants containing exon 2 and that the siRNA targeted towards the E1,3,4 transcript does not target the E2 containing transcripts.

FIG. 35 shows that the siRNA targeted towards exon 4 effectively knocks down functional human AT I R protein expression in human cells and that the siRNA targeted towards exon 2 effectively knocks down functional AT1R protein expression. This data also shows that even though the E1,3,4 transcript is prevalent in HEK293 cells, it does not code for the majority of functional AT1R expression and thus does not knock down much functional AT1R protein expression.

### DETAILED DESCRIPTION OF THE INVENTION

I. Definitions

For convenience, certain terms employed in the specification, examples, and appended claims are collected here.

A “patient” or “subject” to be treated by a disclosed method can be a human or non-human animal.

The term “condition,” as used herein, is intended to include active disorders, e.g., disorders which have manifested their symptoms, and predisposition to a disorder (e.g., the genetic tendency toward a disorder which has not yet manifested itself symptomatically).

The term “expression” with respect to a gene sequence refers to transcription of the gene and, as appropriate, translation of the resulting mRNA transcript to a protein. Thus, as will be clear from the context, expression of a protein coding sequence results from transcription and translation of the coding sequence. A method that decreases the expression of a gene may do so in a variety of ways (none of which are mutually exclusive), including, for example, by inhibiting transcription of the gene, decreasing the stability of the mRNA, or decreasing translation of the mRNA. While not wishing to be bound to a particular mechanism, it is generally thought that siRNA techniques decrease gene expression by stimulating the degradation of targeted mRNA species.

As used herein, the term “nucleic acid” refers to polynucleotides such as deoxyribonucleic acid (DNA) and ribonucleic acid (RNA). The term should also be understood to include, as applicable to the embodiment being described, single-stranded (such as sense or antisense) and double-stranded polynucleotides. The “canonical” nucleotides are adenosine (A), guanosine (G), cytosine (C), thymidine (T), and uracil (U), and include a ribose-phosphate backbone, but the term nucleic acid is intended to include polynucleotides comprising only canonical nucleotides as well as polynucleotides including one or more modifications to the sugar phosphate backbone or the nucleoside. DNA and RNA are chemically different because of the absence or presence of a hydroxyl group at the 2′ position on the ribose. Modified nucleic acids that cannot be readily termed DNA or RNA (e.g., in which an entirely different moiety is positioned at the 2′ position) and nucleic acids that do not contain a ribose-based backbone may be referred to as XNAs. Examples of XNAs are peptide nucleic acids (PNAs) in which the backbone is a peptide backbone, and locked nucleic acids (LNAs) containing a methylene linkage between the 2′ and 4′ positions of the ribose. An “unmodified” nucleic acid is a nucleic acid that contains only canonical nucleotides and a DNA or RNA backbone.

“Small molecule” as used herein, is meant to refer to a compound that has a molecular weight of less than about 5 kD and most preferably less than about 2.5 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures comprising arrays of small molecules, often fungal, bacterial, or algal extracts, which can be screened with any of the assays of the invention.

The term “pharmaceutically acceptable salts” refers to physiologically and pharmaceutically acceptable salts of the compounds of the invention, i.e., salts that retain the desired biological activity of the parent compound and do not impart undesired toxicological effects thereto.

By “recombinant virus” is meant a virus that has been genetically altered, e.g., by the addition or insertion of a heterologous nucleic acid construct into the particle.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo. “RNAi expression vector” (also referred to herein as a “dsRNA-encoding plasmid”) refers to replicable nucleic acid constructs used to express (transcribe) RNA which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell. In general, expression vectors of utility in recombinant DNA techniques are often in the form of “plasmids” which refer to circular double stranded DNA loops which, in their vector form, are not bound to the chromosome. “Plasmid” and “vector” are used herein interchangeably as the plasmid is the most commonly used form of vector. However, the invention is intended to include other forms of expression vectors that serve equivalent functions and become known in the art subsequently hereto.

In the expression vectors, regulatory elements controlling transcription can be generally derived from mammalian, microbial, viral or insect genes. The ability to replicate in a host, usually conferred by an origin of replication, and a selection gene to facilitate recognition of transformants may additionally be incorporated. Vectors derived from viruses, such as retroviruses, adenoviruses, and the like, may be employed.

The term “small interfering RNAs” or “siRNAs” refers to nucleic acids around 19-30 nucleotides in length, and more preferably 21-23 nucleotides in length. The siRNAs are double-stranded, and may include short overhangs at each end. Preferably, the overhangs are 1-6 nucleotides in length at the 3′ end. It is known in the art that the siRNAs can be chemically synthesized, or derive from a longer double-stranded RNA or a hairpin RNA. The siRNAs have significant sequence similarity to a target RNA so that the siRNAs can pair to the target RNA and result in sequence-specific degradation of the target RNA through an RNA interference mechanism. Optionally, the siRNA molecules comprise a 3′ hydroxyl group.

“Hybridization” refers to any process by which a strand of nucleic acid binds with a complementary strand through base pairing. Two single-stranded nucleic acids “hybridize” when they form a double-stranded duplex. The region of double-strandedness can include the full-length of one or both of the single-stranded nucleic acids, or all of one single stranded nucleic acid and a subsequence of the other single stranded nucleic acid, or the region of double-strandedness can include a subsequence of each nucleic acid. Hybridization also includes the formation of duplexes that contain certain mismatches, provided that the two strands are still forming a double stranded helix. “Stringent hybridization conditions” refers to hybridization conditions resulting in essentially specific hybridization.

The term “antisense oligonucleotides” means a sequence of nucleic acids constructed so as to bind to the mRNA encoding a certain protein and thereby prevent translation of the mRNA into protein. Antisense oligonucleotides corresponding to regions of mRNA were synthesized by standard chemical techniques.

As used herein, the terms “transduction” and “transfection” are art-recognized and mean the introduction of a nucleic acid, e.g., an expression vector, into a recipient cell by nucleic acid-mediated gene transfer. “Transformation,” as used herein, refers to a process in which a cell's genotype is changed as a result of the cellular uptake of exogenous DNA or RNA, and, for example, the transformed cell expresses an RNAi construct. A cell has been “stably transfected” with a nucleic acid construct when the nucleic acid construct is capable of being inherited by daughter cells. “Transient transfection” refers to cases where exogenous DNA does not integrate into the genome of a transfected cell, e.g., where episomal DNA is transcribed into mRNA and translated into protein.

II. Screening for Candidate Agents

The candidate agents used in the invention may be pharmacologic agents already known in the art or may be agents not previously known to have pharmacological activity. The agents may be naturally occurring or designed or prepared in the laboratory. They may be isolated from microorganisms, animals, or plants, or may be produced recombinantly, or synthesized by chemical methods known in the art. In some embodiments, candidate agents are identified from small chemical libraries, peptide libraries, or collections of natural products using the methods of the present invention. Tan et al. described a library with over two million synthetic compounds that is compatible with miniaturized cell-based assays (J. Am. Chem. Soc. 120, 8565-8566, 1998). It is within the scope of the present invention that such a library may be used to screen for agents conferring posttranscriptional regulation on AT1R gene using the methods of the invention. There are numerous commercially available compound libraries, such as the Chembridge DIVERSet. Libraries are also available from academic investigators, such as the Diversity set from the NCI developmental therapeutics program.

One basic approach to search for a subject agent is screening of compound libraries. One may simply acquire, from various commercial sources, small molecule libraries that are believed to meet the basic criteria for useful drugs in an effort to identify useful compounds by “brute force.” Screening of such libraries, including combinatorially generated libraries, is a rapid and efficient way to screen a large number of related (and unrelated) compounds for activity. Combinatorial approaches also lend themselves to rapid evolution of potential drugs by the creation of second, third, and fourth generation compounds modeled on active but otherwise undesirable compounds. It will be understood that undesirable compounds include compounds that are typically toxic, but have been modified to reduce the toxicity or compounds that typically have little effect with minimal toxicity and are used in combination with another compound to produce the desired effect.

On the other hand, many useful pharmacological compounds are compounds structurally related to compounds that interact naturally with the targets, which may be the pre-splicing AT1R mRNA, the E2 or ΔE2 transcript, or the RNABPs. Creating and examining the action of such molecules is known as “rational drug design,” and include making predictions relating to the structure of the targets. Thus, it is understood that a subject agent identified by the present invention may be a small molecule inhibitor or any other compound (e.g., polypeptide or polynucleotide) that may be designed through rational drug design starting from known inhibitors of the targets.

The goal of rational drug design is to produce structural analogs of biologically active target compounds. By creating such analogs, it is possible to fashion drugs that are more active or stable than the natural molecules, have different susceptibility to alteration or may affect the function of various other molecules. In one approach, one can generate a three-dimensional structure for molecules like the targets, and then design a molecule for its ability to interact with the targets. Alternatively, one could design a partially functional fragment of the targets (for example, binding, but not affecting the activity of the RNABPs), thereby creating a competitive inhibitor. This could be accomplished by X-ray crystallography, computer modeling, or by a combination of both approaches.

In a particularly preferred embodiment, rational drug design is directed to synthesizing siRNAs that selectively inhibit the expression of ΔE2. The selective inhibition can be based on the unique juncture of RNA present in the ΔE2 transcript but absent from E2.

It is also possible to use antibodies to inform the structure of a target compound or inhibitor. In principle, this approach yields a pharmacore upon which subsequent drug design can be based. It is possible to bypass protein crystallography altogether by generating anti-idiotype antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Other suitable inhibitors include antisense molecules, ribozymes, and antibodies (including single chain antibodies).

It will, of course, be understood that all of the screening methods of the present invention are useful in themselves nonwithstanding the fact that effective candidates may not be found in a particular iteration of the screen. The invention provides methods of screening for such candidates, whether or not an active compound is actually identified.

a. siRNA Technology

RNA interference (RNAi) is a phenomenon describing double-stranded (ds)RNA-dependent gene specific posttranscriptional silencing. Initial attempts to harness this phenomenon for experimental manipulation of mammalian cells were foiled by a robust and nonspecific antiviral defense mechanism activated in response to long dsRNA molecules. Gil et al. Apoptosis 2000, 5:107-114. The field was significantly advanced upon the demonstration that synthetic duplexes of 21 nucleotide RNAs could mediate gene specific RNAi in mammalian cells, without invoking generic antiviral defense mechanisms. Elbashir et al. Nature 2001, 411:494-498; Caplen et al. Proc Natl Acad Sci 2001, 98:9742-9747. As a result, small-interfering RNAs (siRNAs) have become powerful tools to dissect gene function. The chemical synthesis of small RNAs is one avenue that has produced promising results. Numerous groups have also sought the development of DNA-based vectors capable of generating such siRNA within cells. Several groups have recently attained this goal and published similar strategies that, in general, involve transcription of short hairpin (sh)RNAs that are efficiently processed to form siRNAs within cells. Paddison et al. PNAS 2002, 99:1443-1448; Paddison et al. Genes & Dev 2002, 16:948-958; Sui et al. PNAS 2002, 8:5515-5520; and Brummelkamp et al. Science 2002, 296:550-553. These reports describe methods to generate siRNAs capable of specifically targeting numerous endogenously and exogenously expressed genes.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration). Additional modified nucleotides are as follows (this list contains forms that are modified on either the backbone or the nucleoside or both, and is not intended to be all-inclusive): 2′-O-Methyl-2-aminoadenosine; 2′-O-Methyl-5-methyluridine; 2′-O-Methyladenosine; 2′-O-Methylcytidine; 2′-O-Methylguanosine; 2′-O-Methyluridine; 2-Amino-2′-deoxyadenosine; 2-Aminoadenosine; 2-Aminopurine-2′-deoxyriboside; 4-Thiothymidine; 4-Thiouridine; 5-Methyl-2′-deoxycytidine; 5-Methylcytidine; 5-Methyluridine; 5-Propynyl-2′-deoxycytidine; 5-Propynyl-2′-deoxyuridine; N1-Methyladenosine; N1-Methylguanosine; N2-Methyl-2′-deoxyguanosine; N6-Methyl-2′-deoxyadenosine; N6-Methyladenosine; O6-Methyl-2′-deoxyguanosine; and O6-Methylguanosine.

The double-stranded structure may be formed by a single self-complementary nucleic acid strand or two complementary nucleic acid strands. Duplex formation may be initiated either inside or outside the cell. The RNAi construct may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Given the greater uptake of the modified RNAi nucleic acids disclosed herein, it is understood that lower dosing may be employed than is generally used with traditional RNAi constructs. Inhibition is sequence-specific in that nucleotide sequences corresponding to the duplex region of the RNA are targeted for genetic inhibition.

In certain embodiments, the subject RNAi constructs are “small interfering RNAs” or “siRNAs.” These nucleic acids include an antisense RNA strand that is around 19-30 nucleotides in length, and even more preferably 21-23 nucleotides in length, e.g., corresponding in length to the fragments generated by nuclease “dicing” of long double-stranded RNAs. siRNAs may include a sense strand that is RNA, DNA or XNA. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA antisense molecules comprise a 3′ hydroxyl group. Optionally, the sense strand comprises at least 50%, 60%, 70%, 80%, 90% or 100% modified nucleic acids, while the antisense strand is unmodified RNA. Optionally, the sense strand comprises 100% modified nucleic acids (e.g. DNA or RNA with a phosphorothioate modification at every possible position) while the antisense strand is an RNA strand comprising no modified nucleic acids or no more than 10%, 20%, 30%, 40% or 50% modified RNA nucleic acids.

The siRNA molecules of the present invention can be obtained using a number of techniques known to those of skill in the art. For example, the siRNA can be chemically synthesized or recombinantly produced using methods known in the art. For example, short sense and antisense RNA, DNA or XNA oligomers can be synthesized and annealed to form double-stranded structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be introduced into cells, either by passive uptake or a delivery system of choice.

In certain embodiments, an RNAi construct is in the form of a hairpin structure. The hairpin can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci U S A, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell. A hairpin may be chemically synthesized such that a sense strand comprises RNA, DNA or XNA, while the antisense strand comprises RNA. In such an embodiment, the single strand portion connecting the sense and antisense portions should be designed so as to be cleavable by nucleases in vivo, and any duplex portion should be susceptible to processing by nucleases such as Dicer.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, or affinity purification with antibody can be used to purify siRNAs.

In certain preferred embodiments, at least one strand of the siRNA molecules has a 3′ overhang from about 1 to about 6 nucleotides in length, or from 2 to 4 nucleotides in length. More preferably, the 3′ overhangs are 1-3 nucleotides in length. In certain embodiments, one strand has a 3′ overhang and the other strand is blunt-ended or also has an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

An example of siRNAs targeting the RNA juncture between exonsI and 3 unique to the rat ΔE2 has the nucleotide sequence of 5′-CUGGUCAAGUGGAUUUCGAUU-3′ (SEQ ID NO: 1) (with the 3′ overhang of UU) and its complementary strand has the nucleotide sequence of 3′-GGGACCAGUUCACCUAAAGCU-5′ (SEQ ID NO: 2) (with the 3′ overhang of GG). As is known in the art, siRNAs having different nucleotide sequences can be made, and these siRNAs can be screened based on their ability to inhibit the target gene expression. In preferred embodiments, however, the RNA juncture between exons1 and 3 unique to ΔE2 naturally limit the number of possible desired siRNAs. As is known in the art, siRNAs targeting the RNA juncture between exons 1 and 3 unique to other mammalian ΔE2s, e.g., human ΔE2, can be readily made, based on the published E2 and ΔE2 nucleic acid sequences.

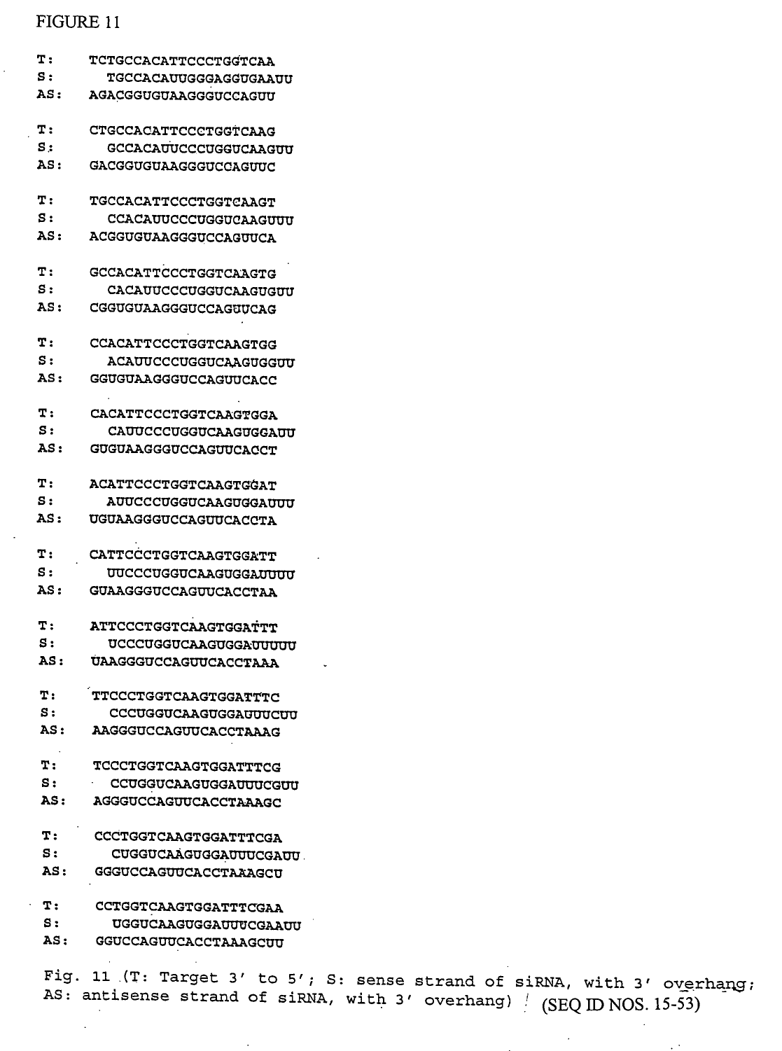

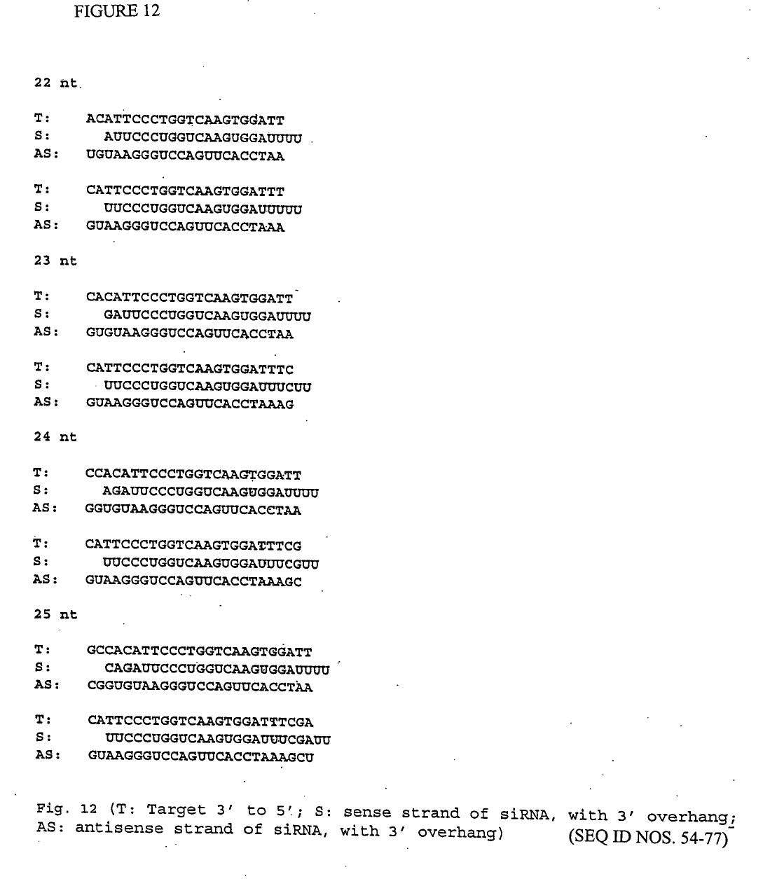

Examples of siRNAs designed to target the RNA juncture between exonsl and 3 unique to the rat ΔE2 are also shown in FIG. 11 and FIG. 12. As is known in the art, siRNAs of nucleotide sequences homologous to these representative sequences can also be used to attenuate the expression of ΔE2 transcript. Alternatively, siRNAs that can specifically hybridize with the target sequences are also contemplated in the present invention. Hybridization can be carried out under wash conditions of 2×SSC at 22° C., and more preferably 0.2 x SSC at 65 ° C, to a target sequence.

As is known in the art, siRNAs generally tolerate mutations in the 5′ end, while the 3′ end exhibited low tolerance (Amarzguioui et al., Nucleic Acids Res. 31(2): 589-95, January 2003). Accordingly, the preferred siRNAs in the present invention are designed to complement the 5′ end of a target sequence, whereas mismatches to the 3′ end of a target sequence can be tolerated. The tolerance to mismatches at the 5′ end of siRNAs, i.e., the 3′ end of target sequences, is especially useful, as single nucleotide polymorphism of AT1R gene is known in the art to be present.

The RNAi constructs of the invention may also be admixed, encapsulated, conjugated or otherwise associated with other molecules, molecule structures or mixtures of compounds, as for example, liposomes, polymers, receptor targeted molecules, oral, rectal, topical or other formulations, for assisting in uptake, distribution and/or absorption. The subject RNAi constructs can be provided in formulations also including penetration enhancers, carrier compounds and/or transfection agents.

Representative United States patents that teach the preparation of such uptake, distribution and/or absorption assisting formulations which can be adapted for delivery of RNAi constructs, particularly siRNA molecules, include, but are not limited to, U.S. Pat. Nos. 5,108,921; 5,354,844; 5,416,016; 5,459,127; 5,521,291;51543,158; 5,547,932; 5,583,020; 5,591,721; 4,426,330;4,534,899; 5,013,556; 5,108,921; 5,213,804; 5,227,170;5,264,221; 5,356,633; 5,395,619; 5,416,016; 5,417,978;5,462,854; 5,469,854; 5,512,295; 5,527,528; 5,534,259; 5,543,152; 5,556,948; 5,580,575; and 5,595,756.

The RNAi constructs of the invention also encompass any pharmaceutically acceptable salts, esters or salts of such esters, or any other compound which, upon administration to an animal including a human, is capable of providing (directly or indirectly) the biologically active metabolite or residue thereof. Accordingly, for example, the disclosure is also drawn to RNAi constructs and pharmaceutically acceptable salts of the siRNAs, pharmaceutically acceptable salts of such RNAi constructs, and other bioequivalents.

b. Antisense Technology

Another aspect of the invention relates to the use of the isolated nucleic acid in “antisense” therapy. As used herein, “antisense” therapy refers to administration or in situ generation of oligonucleotide probes or their derivatives which specifically hybridize (e.g. bind) under cellular conditions, with the cellular mRNA and/or genomic DNA encoding a target splicing variant of AT1R, preferably ΔE2, so as to inhibit translation of the target splicing variant. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding to oligonucleotide sequences.

An antisense molecule of the present invention can be delivered, for example, as an expression plasmid which, when transcribed in the cell, produces RNA which is complementary to at least a unique portion of a target splicing variant of AT1R, preferably ΔE2. Alternatively, the antisense molecule is an oligonucleotide probe which is generated ex vivo and which, when introduced into the cell, causes inhibition of expression by hybridizing with a target splicing variant of AT1R and/or genomic sequences of an AT1R gene. Such oligonucleotide probes are preferably modified oligonucleotides which are resistant to endogenous nucleases, e.g., exonucleases and/or endonucleases, and are therefore stable in vivo. Exemplary nucleic acid molecules for use as antisense oligonucleotides are phosphoramidite, phosphorothioate, and methylphosphonate analogs of DNA (see also U.S. Pat. Nos. 5,176,996; 5,264,564; and 5,256,775), or peptide nucleic acids (PNAs). Additionally, general approaches to constructing oligomers useful in antisense therapy have been reviewed, for example, by Van der Krol et al. (1988) Biotechniques 6:958-976; and Stein et al. (1988) Cancer Res 48:2659-2668.

Accordingly, the agents, e.g., RNAi molecules, of the invention are useful in therapeutic, diagnostic, and research contexts. In therapeutic applications, the agents are utilized in a manner appropriate for antisense therapy in general. For such therapy, the agents of the invention can be formulated for a variety of routes of administration, including systemic and topical or localized administration. Techniques and formulations generally may be found in Remington's Pharmaceutical Sciences, Meade Publishing Co., Easton, Pa. For systemic administration, injection is preferred, including intramuscular, intravenous, intraperitoneal, and subcutaneous. For injection, the agents of the invention can be formulated in liquid solutions, preferably in physiologically compatible buffers such as Hank's solution or Ringer's solution. In addition, the agents may be formulated in solid form and redissolved or suspended immediately prior to use. Lyophilized forms are also included.

Systemic administration can also be by transmucosal or transdermal means, or the compounds can be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For oral administration, the agents are formulated into conventional oral administration forms such as capsules, tablets, and tonics. For topical administration, the agents of the invention are formulated into ointments, salves, gels, or creams as is generally known in the art.

In addition to use in therapy, the agents of the invention may be used as diagnostic reagents to detect the presence or absence of the target splicing variant (E2 or ΔE2) sequences to which they specifically bind.

Likewise, the antisense molecules of the present invention, by antagonizing the normal biological activity of a target splicing variant (E2 or ΔE2), e.g., by reducing the level of its translation and/or expression, can be used in the manipulation of tissue, e.g. tissue maintenance, differentiation or growth, both in vivo and ex vivo.

Furthermore, the anti-sense techniques (e.g., microinjection of antisense molecules, or transfection with plasmids whose transcripts are anti-sense with regard to a target splicing variant or gene sequence) can be used to investigate the role of alternative splicing in regulating functions of cells where such alternative splicing exists, e.g., aorta, renal cortex, aortic smooth muscles. Such techniques can be utilized in cell culture, but can also be used in the creation of transgenic animals (described infra).

c. Combinatorial Libraries

Compounds suitable for use in the present invention, particularly libraries of variants having various representative classes of substituents, are amenable to combinatorial chemistry and other parallel synthesis schemes (see, for example, PCT WO 94/08051). The result is that large libraries of related compounds, e.g., a variegated library of compounds represented above, can be screened rapidly in high throughput assays in order to identify potential lead compounds, as well as to refine the specificity, toxicity, and/or cytotoxic-kinetic profile of a lead compound. For instance, in vitro translation assays using E2 and ΔE2 can be used to screen a library of the subject compounds for those inhibit the translation of ΔE2 or promote the translation of E2 or both.

Simply for illustration, a combinatorial library for the purposes of the present invention is a mixture of chemically related compounds which may be screened together for a desired property. The preparation of many related compounds in a single reaction greatly reduces and simplifies the number of screening processes which need to be carried out. Screening for the appropriate physical properties can be done by conventional methods.

Diversity in the library can be created at a variety of different levels. For instance, the substrate aryl groups used in the combinatorial reactions can be diverse in terms of the core aryl moiety, e.g., a variegation in terms of the ring structure, and/or can be varied with respect to the other substituents.

A variety of techniques are available in the art for generating combinatorial libraries of small organic molecules to be screened by the methods of the present invention. (See, for example, Blondelle et al. (1995) Trends Anal. Chem. 14:83; the Affymax U.S. Pat. Nos. 5,359,115 and 5,362,899; the Ellman U.S. Pat. No. 5,288,514; the Still et al. PCT publication WO 94/08051; the ArQule U.S. Pat. Nos. 5,736,412 and 5,712,171; Chen et al. (1994) JACS 116:2661; Kerr et al. (1993) JACS 115:252; PCT publications WO92/10092, WO93/09668 and WO91/07087; and the Lerner et al. PCT publication WO93/20242, all incorporated herein by reference in full). Accordingly, a variety of libraries on the order of about 100 to 1,000,000 or more diversomers of the candidate agents can be synthesized and screened for particular activity or property.

In one embodiment, a library of candidate agent diversomers can be synthesized utilizing a scheme adapted to the techniques described in the Still et al., PCT publication WO 94/08051 (incorporated herein in fill), e.g., being linked to a polymer bead by a hydrolyzable or photolyzable group, optionally located at one of the positions of the candidate antagonists or a substituent of a synthetic intermediate. According to the Still et al. technique, the library is synthesized on a set of beads, each bead including a set of tags identifying the particular diversomer on that bead. The bead library can then be mixed with translation reaction mixtures, e.g., in an in vitro translation assay.

Many variations on the above and related pathways permit the synthesis of widely diverse libraries of compounds which may be tested as the subject agents.

d. Screening Assays

There are a variety of assays available for determining the ability of a compound to regulate AT1R mRNA post-transcriptionally that can be performed in high-throughput formats. A compound may inhibit the alternative splicing that specifically results in ΔE2, or promote the alternative splicing that specifically results in E2, or do both. Another type of compound of the present invention selectively inhibits translation of ΔE2, or promotes translation initiation from E2 without affecting the cis-regulatory effect of E2 itself, or does both. A third type of compound of the present invention modulates the activities of RNABPs that interact with the 5′ LS of AT1R transcripts.

A quick, inexpensive and easy assay to run is a binding assay. Binding of a molecule to a target (e.g., AT1R pre-spliced mRNA, E2 or ΔE2 transcripts) may, in and of itself, be inhibitory, due to steric, allosteric or charge-charge interactions. This can be performed in solution or on a solid phase and can be utilized as a first round screen to rapidly eliminate certain compounds before moving into more sophisticated assays. In one embodiment of this kind, the screening of compounds that bind to AT1R pre-spliced mRNA, E2 or ΔE2 transcripts, or RNABPs, or biologically active peptide fragment(s) of RNABPs is provided. In certain embodiments, a biologically active peptide fragment can be a domain that is responsible for RNABPs' interaction with the 5′ LS.

The target AT1R pre-spliced mRNA, E2 or ΔE2 transcripts, or RNABPs (“the targets”) may be free in solution, fixed to a support, expressed in a cell. Either the targets or the compounds subject to screening may be labeled, thereby permitting determining of binding. In another embodiment, the assay may measure the inhibition of binding of the targets to a natural or artificial substrate or binding partner. Competitive binding assays can be performed in which one of the agents included in the assay is labeled. Usually, the targets will be the labeled species, decreasing the chance that the labeling will interfere with the binding moiety's function. One may measure the amount of free label versus bound label to determine binding or inhibition of binding.

In certain embodiments, an in vitro translation assay may be followed by the binding assay as described above to further test the selected compound's ability to regulate AT1R mRNA post-transcriptionally. Many in vitro translation assay techniques are well known in the art. General aspects of in vitro translation assays are described in U.S. Pat. Nos. 4,668,624 and 5,434,079, which are incorporated by reference herein in full. These references describe in vitro translation assays conducted for producing proteins other than AT1Rs in the present invention. Many of the components of the in vitro translation assays can nevertheless be modified according to principles well understood in the art for use in the assays for identifying the candidate agents of the present invention.

In certain embodiments, in vitro splicing assays are conducted. AT1R gene is used as a template in combination with a reporter gene, for example, luciferase, and candidate agents will be screened for their ability to decrease the amount of exon 2 removed by splicing, i.e., resulting in an increase of E2 variants or decrease of ΔE2 variant or both. Alternatively, part of the AT1R gene can be used as a template in the in vitro splicing assay.

In addition to cell-free assays, compounds can also be tested in cell-based assays. Various cell lines can be utilized for screening of the candidate agents, e.g., the rat aortic smooth muscle cell line A10. As is known in the art, cell lines expressing AT1Rs can be created via transfections with nucleic acids encoding AT1R.

Depending on the assay, culture may be required. The cell may then be examined by virtue of a number of different physiologic assays, e.g., reporter gene activities, assays indicating AT1R expression levels such as measuring angiotensin II-induced inositol phosphate production and/or AT1R-specific binding by angiotensin II.

In one embodiment, cells which express AT1R can be contacted with a test compound/agent of interest appropriately formulated based on its biochemical nature, with the assay scoring for, e.g., the density of AT1R expressed on the surface of the cells in the presence of the test compound/agent.

The present invention also contemplates the use of various animal models. Transgenic animals may be created with constructs that permit ectopic AT1R expression, and activity of the AT1R to be controlled and monitored. The generation of these animals can be based on techniques well known in the art. In a preferred embodiment, hypertensive animal models such as DS rats can be used, to screen for test compounds that can maintain or decrease glomerular AT1R density in response to high salt diet.

e. Transgenic Animals:

The present invention is further directed to a transgenic non-human eukaryotic animal, preferably a rodent, such as a mouse or other animal capable of developing detectable characteristics from the expression of an RNAi molecule of the present invention. The RNAi molecule is introduced into the animal, or an ancestor of the animal, at an embryonic stage, preferably the one cell, or fertilized oocyte stage and generally not later than about the 8-cell stage. The zygote or embryo is then developed to term in a pseudo-pregnant foster female. The plasmid DNA is introduced into an animal embryo so as to be chromosomally incorporated in a state which results in super-endogenous expression of its corresponding RNA molecule, e.g., an siRNA targeting ΔE2, an antisenese RNA targeting ΔE2, an RNA encoding E2 or ΔE2.

Transgenic mammals are prepared in a number of ways. A transgenic organism is one that has an extra or exogenous fragment of DNA in its genome. In order to achieve stable inheritance of the extra or exogenous DNA fragment, the integration event must occur in a cell type that can give rise to functional germ cells, either sperm or oocytes. Two animal cell types that can form germ cells and into which DNA can be introduced readily are fertilized egg cells and embryonic stem cells. Embryonic stem (ES) cells can be returned from in vitro culture to a “host” embryo where they become incorporated into the developing animal and can give rise to transgenic cells in all tissues, including germ cells. The ES cells are transfected in culture and then the mutation is transmitted into the germline by injecting the cells into an embryo. The animals carrying mutated germ cells are then bred to produce transgenic offspring.

A preferred method for making the subject transgenic animals is by zygote injection. This method is described, for example in U.S. Pat. No. 4,736,866. The method involves injecting DNA into a fertilized egg, or zygote, and then allowing the egg to develop in a pseudo-pregnant mother. The zygote can be obtained using male and female animals of the same strain or from male and female animals of different strains. The transgenic animal that is born is called a founder, and it is bred to produce more animals with the same DNA insertion. In this method of making transgenic animals, the new DNA typically randomly integrates into the genome by a non-homologous recombination event. One to many thousands of copies of the DNA may integrate at one site in the genome.

In a preferred embodiment, a vector (e.g., a retroviral vector) comprising a desired transgene (e.g., encoding an RNAi molecule of the present invention) is introduced to an oocyte by microinjection. As is known in the art, the retroviral vector comprising the transgene will be integrated into the oocyte genome and the transgene will be expressed.

In a preferred embodiment, the transgene comprises a hairpin RNA of the present invention. In a preferred embodiment, the hairpin RNA targets the juncture between exons 1 and 3 of AT1R. In a preferred embodiment, the hairpin RNA expression is driven by a single promoter.

In another preferred embodiment, the transgene comprises a double-strand siRNA. In a preferred embodiment, the siRNA targets the juncture between exons 1 and 3 of AT1R. In a preferred embodiment, the siRNA expression is driven by two promoters from opposite directions such that both strands of the siRNA will be expressed.

Generally, the DNA comprising a transgene is injected into one of the pronuclei, usually the larger male pronucleus. The zygotes are then either transferred the same day, or cultured overnight to form 2-cell embryos and then transferred into the oviducts of the pseudo-pregnant females. The animals born are screened for the presence of the desired integrated DNA. By a pseudo-pregnant female is intended a female in estrous who has mated with a vasectomized male; she is competent to receive embryos but does not contain any fertilized eggs. Pseudo-pregnant females are important for making transgenic animals since they serve as the surrogate mothers for embryos that have been injected with DNA or embryonic stem cells.

Putative founders are screened for the presence of the transgene by PCR analysis of tail DNA as described in the Example 6. Transgene expression can be initially evaluated by RNA analysis using the Northern blot technique. Preferably, the ratio of E2 to ΔE2 transcripts or total AT1R mRNA is increased in renal tissue of transgenic but not control mice. To ascertain the expression of AT1R at the cell surface, binding experiments can be performed using transgenic and control glomeruli. To this end, labeled angiotensin II radioligand can be employed.

The founder animals can be used to produce stable lines of transgenic animals that superexpress the RNAi as desired. For ease of propagation, male founder mice are preferred. The animals are observed clinically. Analyses of transgene copy number (to exclude multiple transgene insertion sites), total mRNA expression, ratio of the splicing variants and protein expression in these animals are also performed. These studies may provide information about the age of onset of illness, the duration of illness, the penetrance of the phenotype, the range of pathologic findings and the dependence of phenotype upon levels of protein expression.

The present invention also contemplates creating transgenic animals by homologous recombination. The term “homologous recombination” refers to the process of DNA recombination based on sequence homology of nucleic acid sequences in a construct with those of a target sequence, such as a target allele, in a genome or DNA preparation. Accordingly, the nucleic acid sequences present in the construct are identical or highly homologous, that is, they are more than 60%, preferably more than 70%, highly preferably more than 80%, and most preferably more than 90% sequence identity to a target sequence located within a cell genome. In a particular embodiment, the homologous recombination vector has 95%-98% sequence identity to a target sequence located within a cell genome.

In a preferred embodiment, the subject invention provides a construct which, by homologous recombination with a genomic DNA, alters the level of AT1R splicing variants present in the cells.

In preferred embodiments, the nucleotide sequence used as the construct can be comprised of (1) DNA from some portion of the endogenous AT1R gene (exon sequence, intron sequence, promoter sequences, etc.) which direct recombination and (2) heterologous transcriptional regulatory sequence(s) which is to be operably linked to the coding sequence for the genomic AT1R gene upon recombination of the construct. For use in generating cultures of AT1R producing cells, the construct may further include a reporter gene to detect the presence of the knockout construct in the cell.

The construct is inserted into a cell, and integrates with the genomic DNA of the cell in such a position so as to provide the heterologous regulatory sequences in operative association with the native AT1R gene. Such insertion occurs by homologous recombination, i.e., recombination regions of the construct that are homologous to the endogenous AT1R gene sequence hybridize to the genomic DNA and recombine with the genomic sequences so that the construct is incorporated into the corresponding position of the genomic DNA.

The terms “recombination region” or “targeting sequence” refer to a segment (i.e., a portion) of a construct having a sequence that is substantially identical to or substantially complementary to a genomic gene sequence, e.g., including 5′ flanking sequences of the genomic gene, and can facilitate homologous recombination between the genomic sequence and the targeting transgene construct.

As used herein, the term “replacement region” refers to a portion of a construct which becomes integrated into an endogenous chromosomal location following homologous recombination between a recombination region and a genomic sequence. In a preferred embodiment, the replacement region of the present invention encodes E2. In a particularly preferred embodiment, the E2 of the replacement region is not subject to alternative splicing that removes exon 2 in the transgenic animal. In another aspect of the invention, the replacement region encodes ΔE2, in situations, e.g. where higher expression of AT1R may be desirable.

The heterologous regulatory sequences, e.g., which are provided in the replacement region, can include one or more of a variety elements, including: promoters (such as constitutive or inducible promoters), enhancers, negative regulatory elements, locus control regions, transcription factor binding sites, or combinations thereof. Promoters/enhancers which may be used to control the expression of the targeted gene in vivo include, but are not limited to, the cytomegalovirus (CMV) promoter/enhancer (Karasuyama et al., 1989, J. Exp. Med., 169:13), the human P-actin promoter (Gunning et al. (1987) PNAS 84:4831-4835), the glucocorticoid-inducible promoter present in the mouse mammary tumor virus long terminal repeat (MMTV LTR) (Klessig et al. (1984) Mol. Cell Biol. 4:1354-1362), the long terminal repeat sequences of Moloney murine leukemia virus (MuLV LTR) (Weiss et al. (1985) RNA Tumor Viruses, Cold Spring Harbor Laboratory, Cold Spring Harbor, N.Y.), the SV40 early or late region promoter (Bemoist et al. (1981) Nature 290:304-310; Templeton et al. (1984) Mol. Cell Biol., 4:817; and Sprague et al. (1983) J. Virol., 45:773), the promoter contained in the 3′ long terminal repeat of Rous sarcoma virus (RSV) (Yamamoto et al., 1980, Cell, 22:787-797), the herpes simplex virus (HSV) thymidine kinase promoter/enhancer (Wagner et al. (1981) PNAS 82:3567-71), and the herpes simplex virus LAT promoter (Wolfe et al. (1992) Nature Genetics, 1:379-384).

In still other embodiments, the replacement region merely deletes a negative transcriptional control element of the native AT1R gene, e.g., to activate expression, or ablates a positive control element, e.g., to inhibit expression of the targeted gene.

The animals of the present invention can be used as tester animals for materials of interest, e.g. agents to prevent and/or treat angiotensin II-related diseases. An animal is treated with the material of interest, and a reduced incidence or delayed onset of such a disease, e.g., hypertension, as compared to untreated animals, is detected as an indication of protection and/or response to treatment.

The animals of the invention may also be used as models for the molecular mechanism of angiotensin 11-related diseases.

f. Gene Therapy:

This invention also provides expression vectors containing a nucleic acid encoding an RNAi of the present invention, e.g., siRNAs targeting the juncture between exons 1 and 3 of AT1R, operably linked to at least one transcriptional regulatory sequence. Operably linked is intended to mean that the nucleotide sequence is linked to a regulatory sequence in a manner which allows expression of the nucleotide sequence. Regulatory sequences are art-recognized and are selected to direct expression of the subject proteins. Accordingly, the term transcriptional regulatory sequence includes promoters, enhancers and other expression control elements. Such regulatory sequences are described in Goeddel; Gene Expression Technology: Methods in Enzymology 185, Academic Press, San Diego, Calif. (1990). For instance, any of a wide variety of expression control sequences, sequences that control the expression of a DNA sequence when operatively linked to it, may be used in these vectors to express DNA sequences encoding the polypeptides of this invention. Such useful expression control sequences, include, for example, a viral LTR, such as the LTR of the Moloney murine leukemia virus, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage lambda., the control regions for fd coat protein, the promoter for 3-phosphoglycerate kinase or other glycolytic enzymes, the promoters of acid phosphatase, e.g., PhoS, the promoters of the yeast a-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other proteins encoded by the vector, such as antibiotic markers, should also be considered.

Moreover, the gene constructs of the present invention can also be used to deliver nucleic acids encoding an RNAi molecule of the present invention, or a target splicing variant, E2 or ΔE2. Thus, another aspect of the invention features expression vectors for in vivo or in vitro transfection and translation of AT1R splicing variants in particular cell types.

Expression constructs of a target splicing variant (E2 or ΔE2) may be administered in any biologically effective carrier, e.g. any formulation or composition capable of effectively delivering the recombinant gene to cells in vivo or in vitro. Approaches include insertion of the subject gene in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g., antibody-conjugated), polylysine conjugates, gramacidin S, artificial viral envelopes or other such intracellular carriers, as well as direct injection of the gene construct or CaPO₄ precipitation. One of skill in the art can readily select from amongst available vectors and methods of delivery in order to optimize transfection into a particular cell type or under particular conditions.

A preferred approach for introduction of nucleic acid into a cell is by use of a viral vector containing nucleic acid, e.g., a cDNA, encoding an RNAi molecule of the present invention, or a target splicing variant (E2 or ΔE2). Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up the viral vector.

Retrovirus vectors and adeno-associated virus vectors are generally understood to be the recombinant gene delivery system of choice for the transfer of exogenous genes. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76: 271). Thus, recombinant retrovirus can be constructed in which part of the retroviral coding sequence (gag, pol, env) has been replaced by nucleic acid encoding one of the subject proteins rendering the retrovirus replication-defective. The replication-defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include ψCrip, ψCre, ψ2 and ψAm. Retroviruses have been used to introduce a variety of genes into many different cell types, including neuronal cells, in vitro and/or in vivo (see for example Eglitis, et al. (1985) Science 230: 1395-1398; Danos and Mulligan (1988) Proc. Natl. Acad. Sci. USA 85: 6460-6464; Wilson et al. (1988) Proc. Natl. Acad. Sci. USA 85: 3014-3018; Armentano et al. (1990) Proc. Natl. Acad. Sci. USA 87: 6141-6145; Huber et al. (1991) Proc. Natl. Acad. Sci. USA 88: 8039-8043; Ferry et al. (1991) Proc. Natl. Acad. Sci. USA 88: 8377-8381; Chowdhury et al. (1991) Science 254: 1802-1805; van Beusechem et al. (1992) Proc. Natl. Acad. Sci. USA 89: 7640-7644; Kay et al. (1992) Human Gene Therapy 3: 641-647; Dai et al. (1992) Proc. Natl. Acad. Sci. USA 89: 10892-10895; Hwu et al. (1993) J. Immunol. 150: 4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications W093/25234 and W094/06920). For instance, strategies for the modification of the infection spectrum of retroviral vectors include: coupling antibodies specific for cell surface antigens to the viral env protein (Roux et al. (1989) PNAS 86: 9079-9083; Julan et al. (1992) J. Gen Virol 73: 3251-3255; and Goud et al. (1983) Virology 163: 251-254); or coupling cell surface receptor ligands to the viral env proteins (Neda et al. (1991) J Biol Chem 266: 14143-14146). Coupling can be in the form of the chemical cross-linking with a protein or other variety (e.g., lactose to convert the env protein to an asialoglycoprotein), as well as by generating fusion proteins (e.g., single-chain antibody/env fusion proteins). This technique, while useful to limit or otherwise direct the infection to certain tissue types, can also be used to convert an ecotropic vector in to an amphotropic vector.

Moreover, use of retroviral gene delivery can be further enhanced by the use of tissue- or cell-specific transcriptional regulatory sequences which control expression of the gene of the retroviral vector.

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See for example Berkner et al. (1988) BioTechniques 6: 616; Rosenfeld et al. (1991) Science 252: 431-434; and Rosenfeld et al. (1992) Cell 68: 143-155. Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses can be advantageous in certain circumstances in that they can be used to infect a wide variety of cell types, including airway epithelium (Rosenfeld et al.(1992) cited supra), endothelial cells (Lemarchand et al. (1992) Proc. Natl. Acad. Sci. USA 89: 6482-6486), hepatocytes (Herz and Gerard (1993) Proc. Natl. Acad. Sci. USA 90: 2812-2816) and muscle cells (Quantin et al. (1992) Proc. Natl. Acad. Sci. USA 89: 2581-2584). Furthermore, the virus particle is relatively stable and amenable to purification and concentration, and as above, can be modified so as to affect the spectrum of infectivity.

Yet another viral vector system useful for delivery a desired RNAi molecule or nucleic acid encoding a target splicing variant (E2 or ΔE2) in the present invention is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. Curr. Topics in Micro. and Imnunol. (1992) 158: 97-129). It is also one of the few viruses that may integrate its DNA into non-dividing cells, and exhibits a high frequency of stable integration (see for example Flotte et al. (1992) Am. J. Respir. Cell. Mol. Biol. 7: 349-356; Samulski et al. (1989) J. Virol. 63: 3822-3828; and McLaughlin et al. (1989) J.Virol. 62: 1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5: 3251-3260 can be used to introduce DNA into cells. A variety of nucleic acids have been introduced into different cell types using AAV vectors (see for example Hermonat et al. (1984) Proc. Natl. Acad. Sci. USA 81: 6466-6470; Tratschin et al. (1985) Mol. Cell. Biol. 4: 2072-2081; Wondisford et al. (1988) Mol. Endocrinol. 2: 32-39; Tratschin et al. (1984) J. Virol. 51: 611-619; and Flotte et al. (1993) J. Biol. Chem. 268: 3781-3790).

The above cited examples of viral vectors are by no means exhaustive. Herpes-simplex viral vectors and lentiviral vectors are just two additional types of viral vectors which can be used in the present invention.

In addition to viral transfer methods, such as those illustrated above, non-viral methods can also be employed to cause expression of an exogenous nucleic acid of the present invention in cells or animals. Most nonviral methods of gene transfer rely on normal mechanisms used by cells for the uptake and intracellular transport of macromolecules. In preferred embodiments, non-viral gene delivery systems of the present invention rely on endocytic pathways for the uptake of an exogenous nucleic acid, e.g., an RNAi molecule of the present invention, by the targeted cell. Exemplary gene delivery systems of this type include liposomal derived systems, poly-lysine conjugates, and artificial viral envelopes.

III. Pharmaceutical Compositions and Uses Thereof

In another aspect, the present invention provides pharmaceutical preparations comprising, as an active ingredient, an agent capable of regulating AT1R posttranscriptionally such as described herein, formulated in an amount sufficient to alleviate, in vivo, high blood pressure or other biological consequences of aberrant angiotensin-mediated signaling.

The present invention also pertains to pharmaceutical compositions comprising the therapeutic agents identified by methods described herein. A therapeutic agent of the present invention can be formulated with a physiologically acceptable medium to prepare a pharmaceutical composition. The particular physiological medium may include, but is not limited to, water, buffered saline, polyols (e.g., glycerol, propylene glycol, liquid polyethylene glycol) and dextrose solutions. The optimum concentration of the active ingredient(s) in the chosen medium can be determined empirically, according to well known procedures, and will depend on the ultimate pharmaceutical formulation desired. Preferably, the composition is non-pyrogenic, i.e., does not substantially elevate the body temperature of a patient to whom it is administered.

One aspect of the invention is drawn to RNAi constructs and siRNAs, pharmaceutically acceptable salts of such constructs, and other bioequivalents. Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,NI-dibenzylethylenediamine, chloroprocaine, choline, diethanolamine, dicyclohexylamine, ethylenediamine, N-methylglucamine, and procaine (see, for example, Berge et al., “Pharmaceutical Salts,“J. of Pharma Sci., 1977, 66,1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the preparations of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids.

For siRNA oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalene disulfonic acid, polygalacturonic acid, and the like; and (d) salts formed from elemental anions such as chlorine, bromine, and iodine.

Methods of introducing therapeutic agents at the site of treatment include, but are not limited to, intradermal, intramuscular, intraperitoneal, intravenous, subcutaneous, oral and intranasal. Other suitable methods of introduction can also include rechargeable or biodegradable devices and slow release polymeric devices. The pharmaceutical compositions of this invention can also be administered as part of a combinatorial therapy with other agents, or with other treatment methods.

The agents for use in the subject method may be conveniently formulated for administration with a biologically acceptable medium, such as water, buffered saline, polyol (for example, glycerol, propylene glycol, liquid polyethylene glycol and the like) or suitable mixtures thereof. The optimum concentration of the active ingredient(s) in the chosen medium can be determined empirically, according to procedures well known to medicinal chemists. As used herein, “biologically acceptable medium” includes any and all solvents, dispersion media, and the like which may be appropriate for the desired route of administration of the pharmaceutical preparation. The use of such media for pharmaceutically active substances is known in the art. Except insofar as any conventional media or agent is incompatible with the activity of the subject inhibitor agents, its use in the pharmaceutical preparation of the invention is contemplated. Suitable vehicles and their formulation inclusive of other proteins are described, for example, in the book Remington's Pharmaceutical Sciences (“Remington's Pharmaceutical Sciences,“Mack Publishing Company, Easton, Pa., U.S.A., 1985). These vehicles include injectable “deposit formulations.”

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of an active ingredient at a particular target site.

The preparations of the present invention may be given orally, parenterally, topically, or rectally. They are of course given by forms suitable for each administration route. For example, they are administered in tablets or capsule form, by injection, inhalation, eye lotion, ointment, suppository, controlled release patch, etc., administration by injection, infusion or inhalation; topical by lotion or ointment; and rectal by suppositories.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrastemal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

Regardless of the route of administration selected, the compounds of the present invention, which may be used in a suitable hydrated form, and/or the pharmaceutical compositions of the present invention, are formulated into pharmaceutically acceptable dosage forms such as described below or by other conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular inhibitor agent employed, the age, sex, weight, condition, general health and prior medical history of the patient being treated, and like factors well known in the medical arts.

The term “treatment” is intended to encompass also prophylaxis, therapy, and cure.

Treatment of these animals with test compounds will involve administration of the compound, in an appropriate form, to the animal. Administration will be by a route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated are systemic intravenous injection and regional administration via blood or lymph supply.

The patient receiving this treatment is any animal in need, including primates, in particular humans.

Accordingly, the present invention relates to a new method of preventing and/or treating cardiovascular diseases and complications by pharmacological interference with the RAS using an agent that regulate AT1R post-transcriptionally.

In one embodiment, the present invention relates to use of an agent capable of post-transcriptional regulation of AT1R in the manufacture of a medicament for the prophylactic and/or therapeutic treatment of angiotensin II-related diseases. Such diseases may include, without limitation, hypertension, cardiac hypertrophy, myocardial infarction, normal tension glaucoma, disorders on account of neurological pathogenesis, other cardiovascular complications including stroke, vascular access dysfunction and amputations, and cardiovascular complications encountered during or between dialysis of a patient in need of such dialysis.

A further embodiment of the invention provides a method for prophylactic and/or therapeutic treatment of a patient having or being at high risk for an angiotensin II-related disease, comprising administering to the patient having such a disease or being at such a risk a therapeutically effective amount of the medicament or pharmaceutical preparation of the present invention. Such diseases may include, without limitation, hypertension, cardiac hypertrophy, myocardial infarction, normal tension glaucoma, disorders on account of neurological pathogenesis, other cardiovascular complications including stroke, vascular access dysfunction and amputations, and cardiovascular complications encountered during or between dialysis of a patient in need of such dialysis.

The following materials and methods were used in carrying out the work described herein. The invention now being generally described, it will be more readily understood by reference to the following examples, which are included merely for purposes of illustration of certain aspects and embodiments of the present invention, and are not intended to limit the invention.

### EXAMPLES

### Example 1

### In Vitro Translation

ΔE2 (ΔE2) and E2 (E2) cDNA were subcloned into the pcDNA5/FRT expression vector (Invitrogen). ΔE2 and E2 plasmid DNA was linearized by Xho I digestion. After confirming the digestion was complete on an agarose gel, linearized DNA was purified by the PCR purification system (Qiagen), then in vitro transcribed into capped RNA (T7 mMessage mMachine, Ambion). Transcribed RNAs were quantified using the Ribogreen RNA quantitation kit (Molecular Probes). RNA (0.5-2 [g) was translated in wheat germ extracts in the presence of [35S] Methionine (Promega). The translated protein was analyzed by SDS-gel electrophoresis. Protein size was determined by comparison with broad-range prestained SDS-PAGE markers. Autoradiograms of SDS gels were quantified by phosphorimaging and expressed in arbitrary units.

### Example 2

### A10 Cell Culture and Transient Transfections

A10 cells were cultured in Dulbecco's Modified Essential Medium (DMEM) supplemented with 2 mM L-glutamine, 10% fetal bovine serum and antibiotics (100 IU/ml penicillin, 100 μg/ml streptomycin) (DMEM media). When cells were 60-75% confluent, 20 μg of ΔE2- or E2-plasmid DNA per 100 mm dish was transiently transfected by the calcium phosphate method (Calcium phosphate transfection system, Invitrogen).

### Example 3

### AT1R Radioligand-Binding

After washing and pelleted the cells in phosphate buffered saline at 4° C., A10 cell membranes were prepared as described previously. After being re-suspended in Homogenization buffer [10 mM Tris buffer (pH 7.2) containing 0.32 M sucrose, 2 mM EDTA, and 3 mM MgCl₂], the cells were briefly homogenized with a polytron (PT-10, Brinkmann Instruments). The homogenate was centrifuged at 1000× g for 5 min and the resulting supernatant, at 44,000× g for 65 min. The final pellet was re-suspended in Binding buffer [10 mM Tris (pH 7.2) containing 3 mM MgCl₂] and the protein content determined using the Bio-Rad DC protein assay. Membrane protein was adjusted to the appropriate concentration in Binding buffer supplemented with 0.2% bovine serum albumin and stored at −80° C. until use in the radioligand binding assay. Glomeruli were isolated as described previously18. Both A10 membranes (15 μg protein/tube) and isolated glomeruli (5 μg protein/tube) were incubated for 1-2 h at room temperature with increasing concentrations of ¹²⁵I-[Sar1,Ile8] angiotensin II (Peptide Radioiodination Ctr, Pullman, Wash.) in the presence of 1 μM PD-123319, an AT2R antagonist (so only rAT1R expression was measured). Binding reactions were terminated by rapid filtration through a Brandel cell harvester. Quantitation: Bound radioligand was measured in a γ-spectrophotometer. Specific AT1 receptor binding was defined as the total amount of radioligand bound minus the nonspecific binding, defined as the amount bound in the presence of 200 nM Angiotensin II (100× Kd for angiotensin II). Data points were obtained in triplicate. Kd and Bmax values from Scatchard plots were determined using the nonlinear regression analysis program, PRISM.

### Example 4

### Inositol Phosphate Assay

A10 cells were cultured to 65% confluence in 24-well plates in DMEM media before being transiently transfected with rAT1a E2 or ΔE2 plasmid DNA (using the same ratio of DNA/cells for each transcript). After 2 days, the cells were treated for 16 hours with DMEM media containing 3 μCi/ml myo-[³H]-inositol (Amersham). After washing twice, the cells were preincubated with 500 μl of DMEM media supplemented with 10 mM LiCl for 15 min before stimulation with Angiotensin II (10⁻¹⁰ to 10⁻⁶ M) for 20 min at 37 ° C. The incubation was terminated by adding 750 μl/well of ice-cold 10 mM formic acid, followed by a 30 min incubation on ice. [³H]Inositol phosphates were eluted from ion exchange columns (AG-1-X8 resin, 200-400 mesh, BioRad) using 0.1 M formic acid/0.8 M ammonium formate. Radioactive IP fractions were counted in a Beckman scintillation counter.

### Example 5

### Animal Models

Adult female DS and DR rats (175-200 g) were maintained on a phytoestrogen-free, high sodium diet (NaCl =7.6%; Harlan) with free access to water for 4 weeks before mean arterial pressure (MAP), glomerular rAT1R density and expression levels of rAT1aR splice variants were determined.

Mean Arterial Pressure: Animals were anesthetized with Inactin (100 mg/kg, ip) and catheters were placed in the carotid artery for MAP measurements essentially.

### Example 6

### Real-Time PCR

Total RNA was extracted using TRIzol reagent (Life Technologies). First strand cDNA was made from total RNA using the AMV reverse transcriptase system (Promega) with random hexamers. Quantitations of specific mRNAs and 18S rRNA (for control) were performed by real-time PCR using the ABI Prism 7700 Sequence Detection System (Perkin Elmer Applied Biosystems). The PCR reaction mixture consisted of RNase free water, TaqMan Universal PCR Master Mix (Perkin Elmer Applied Biosystems) and 300 nM specific primers and 10 μM probe (Forward primers: 119F (E2), 5′-CCA CAT TCC CTG AGT TAA CAT ATG A-3′ (SEQ ID NO: 3) and 114F (ΔE2), 5′-CTC TGC CAC ATT CCC TGG TC-3′ (SEQ ID NO: 4); Reverse primer: 310R (E2 & ΔE2), 5′-TCT TTT GAT ACC ATC TTC AGC AGA A-3′) (SEQ ID NO: 5); and Probe: 232T (E2 & ΔE2), 6 FAM-TCG AAT AGT GTC TGA GAC CAA CTC AAC CCA-TAMRA) (SEQ ID NO: 6), and cDNA samples. PCR conditions were optimized for the probe (232T) and both sets of primers (119F & 310R and 114F & 310R) using control cDNAs. The expression of mRNA and 18S rRNA in each sample was quantitated using respective primers. PCR reactions without reverse transcription were included to control for contamination by genomic DNA. The standard curves for 18S rRNA and mRNA were made by a series of ten times dilutions (5³, 5⁴, 5⁵, 5⁶, 5⁷, and 58) of the cDNA. The standard curves were calculated based on the control values.

### Example 7

### ΔE2 RNA is Translated into rAT1R Protein More Efficiently than E2

ΔE2 and E2 plasmid DNA (pcDNA5/FRT) was linearized by Xho I digestion then in vitro transcribed into capped RNA. RNA (0.5-2 μg) was translated in wheat germ extracts in the presence of [35S] methionine and analyzed by phosphorimaging of autoradiograms of SDS gels. These in vitro studies revealed that 1.8-fold more rAT1R protein (40 KDa) was synthesized by the ΔE2 transcript compared to the E2 variant.

### Example 8

### The Density of rAT1aRs is Markedly Higher in A10 Cells Transfected with ΔE2 Compared to E2 Plasmid DNA

The A10 rat aortic smooth muscle cell line was transiently transfected by calcium phosphate with ΔE2 or E2 plasmid DNA for 2 days before membranes were isolated for radioligand binding analysis. These transfection experiments revealed that the rAT1R density (Bmax) was 40% higher in A10 cells transfected with the ΔE2 plasmid compared to the E2.

### Example 9

### rAT1aR signaling is markedly higher in A10 cells transfected with ΔE2 compared to E2 plasmid DNA

A10 cells were transiently transfected with ΔE2 or E2 plasmid cDNA for two days before loading cells with [³H]-inositol. Cells transfected with the ΔE2 plasmid accumulated 33% more inositol phosphates than E2 transfected cells after stimulation with 100 nM angiotensin II for 20 min (angiotensin II/basal: ΔE2, 1.8±0.2; E2, 1.35±0.2, n=4, p<0.05).

### Example 10

### AT1R Densities are Elevated in the Hypertensive DS Rat Kidney

Female DS and DR rats were maintained on a normal salt (NS, 0.4% NaCl) and high salt (HS, 7.6% NaCl) diet for 4 weeks. The HS diet significantly increased the MAP in the DS rats compared to the NS diet but had no significant effects on MAP in the DR rats. No significant differences in MAP were found between the DS and DR animals maintained on the NS diet. On the HS diet, glomerular AT1R density was significantly increased (by 50%) in the DS rats maintained on the HS diet compared to the DR animals and by 15% compared to the DS animals maintained on the NS diet. No significant differences in AT1R expression were found between the DR rats maintained on the NS and HS diets. Furthermore, on the NS diet, the DS rats had a significantly higher level of glomerular AT1R density compared to the DR animal maintained on either the HS or NS diets. These findings were similar to AT1R binding to membranes prepared from renal cortex, although AT1R densities were 10-fold less in whole renal cortex preparations compared to isolated glomeruli preparations.

### Example 11

### The Hypertensive DS Rat Expresses a Higher Percentage of Renal ΔE2/Total AT1aR mRNA Compared to Normotensive DR and DS Rats

Total RNA was isolated from the renal cortex of DR and DS animals maintained on NS and HS diets and reverse transcribed before real-time PCR was performed using primers specific to the ΔE2 and E2 splice variants. Two rAT1aR alternative splice transcripts (ΔE2 and E2) were amplified. Although no significant differences were observed in the total levels of AT1aR mRNA among all four animal groups (data not shown), the ratio of ΔE2/E2 was 57% higher in the hypertensive DS rat compared to the normotensive DR animal maintained on the HS diet. This increase in the ratio of the splice variant lacking E2 was not due to increased ΔE2 expression but rather a decrease in the E2 transcript.

### Example 12

### Investigation of Functional Differences Between Two Possible Splice Variants of the AT1aR (E1,3 and E1,2,3) and Examination of the Exonic Composition of the AT1aR in Ang II Target Tissues

Described in this example is an investigation of the functional differences between two possible splice variants of the AT1aR (E1,3 and E1,2,3) in stably transfected Chinese Hamster Ovary (CHO) cells and examination of the exonic composition of the AT1aR in Ang II target tissues.

This example describes the results of an investigation of the effect of E2 on AT1R expression and signaling in Chinese Hamster Ovary (CHO) cells stably transfected with E1,3 or E1,2,3 DNA. AT1R membrane densities (Bmax) were 1.8-fold higher in CHO cells stably expressing the E1,3 variant compared to those expressing E1,2,3 [Bmax (fmol/mg protein): E1,3, 97.6±13 vs E1,2,3, 53.7±1.2; n=3, p<0.05] and Ang II (100 nM) stimulated inositol phosphate production was 5-fold higher [Ang II-basal (cpm): E1,3, 1320±100 vs E1,2,3, 260±16, n=3, p<0.001] in E1,3 compared to E1,2,3 expressing cells. No differences were observed between E1,3 and E1,2,3 mRNA stability. In vitro translation assays revealed that 1.8-fold higher levels of AT1aR protein were obtained from the E1,3 transcript compared to the E1,2,3 variant (Arbitrary units: E1,3, 1.8±0.13 vs E1,2,3, 1.0±0.06; n=3, p<0.001). These results suggest that the presence of exon 2 in the E1,2,3 transcript reduces the expression of functional receptors in the cell membrane by inhibiting the translational efficiency of the AT1aR.

Methods

CHO cell culture and stable transfection: CHO cells were cultured in Dulbecco's Modified Essential Medium (DMEM) supplemented with 2 mM L-glutamine, 10% fetal bovine serum and antibiotics (100 IU/ml penicillin, 100 μg/ml streptomycin) (DMEM media). When cells were 60-75% confluent, 20 μg of E1,3- or E1,2,3-pcDNA/5FRT-expression vector per 100 mm dish was transfected by the Lipofection transfection system (Invitrogen). Individual clones were isolated as described previously (Tian, 1993) using hygromycin for clone selection. AT1R radioligand binding: Cell and tissue membranes were prepared as described previously (Ji et al., 1994; Owonikoko et al., 2004). Membranes prepared from CHO cells (40 μg), heart (100 μg), spleen (100 μg), liver (50 μg) and preglomerular tissue (10 μg) were incubated for 1-2 h at room temperature with increasing concentrations of 125I-[Sar1,Ile8]Ang II (Peptide Radioiodination Ctr, Pullman, Wash.) in the presence of 1 μM PD-123319, an AT2R antagonist (so only AT1R expression was measured) as described (Zheng et al., 2001). Inositol phosphate (IP) assay: CHO cells stably expressing E1,2,3 or E1,3 were cultured to 70% confluence in 24-well plates for 24 h in DMEM media. The cells were then treated for 16 hours with DMEM media containing 3 μCi/ml myo-[3H]-inositol (Amersham). After washing twice, the cells were preincubated with 500 μl of DMEM media supplemented with 10 mM LiCl for 15 min before stimulation with Ang II (100 nM) for 20 min at 37° C. In initial experiments, dose response curves revealed that incubation with 100 nM Ang II yielded maximum levels of Ang II-stimulated IP accumulation. The incubation was terminated by adding 750 μl/well of ice-cold 10 mM formic acid, followed by a 30 min incubation on ice. [3H]Inositol phosphates were eluted from ion exchange columns (AG-1-X8 resin, 200-400 mesh, BioRad) using 0.1 M formic acid/0.8 M ammonium formate. Radioactive IP fractions were counted in a Beckman scintillation counter (Vanderheyden et al., 1999)

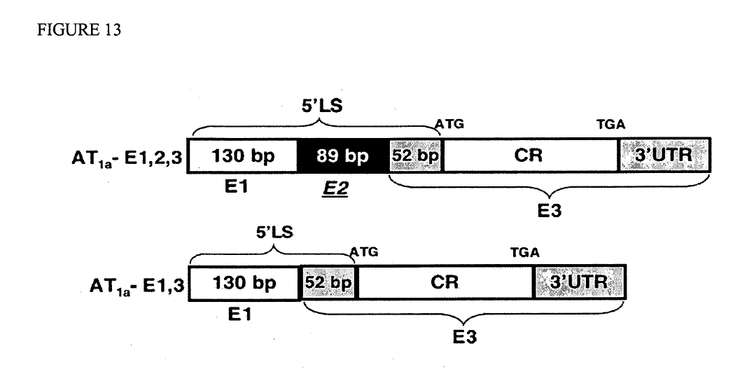

mRNA stability assay: CHO cells expressing E1,2,3 or E1,3 were cultured at 80% confluency in 100 mm dishes in DMEM media. After 24-h culture, cells were treated with the transcription inhibitor, actinomycin D (20 μg/ml) for another 24 h (Ouali et al., 1997). In control studies, Applicants confirmed that gene transcription was blocked under these conditions by measuring the incorporation of [3H]uridine during a 4-hr chase into poly(A)+ RNA fractions (Nickenig and Murphy, 1996). After 0, 2, 6, 12 and 24 h time points, total RNA was isolated and E1,3 and E1,2,3 mRNA were measured by RNA protection assay as previously described (Wu et al., 2003) using a probe based on the Exon 3coding region and thus common to both transcripts (FIG. 13). In brief, the cDNA encoding the rat AT1aR coding region in the pBluescript II vector (Stratagene) was linearized with Acc IEcoR1 and transcribed in vitro with T7 RNA polymerase to yield a 90 380 bp protected cRNA fragment after hybridization with 5 3 μg of total RNA followed by RNase digestion according to the RPA III protocol (Ambion). The probe for actin was generated from pTRI-Actin-Mouse with T7 RNA polymerase and yielded a 245 bp cRNA fragment. Radioactive signals were detected by a phosphoimager after electrophoresis on a 5% acrylamide gel.

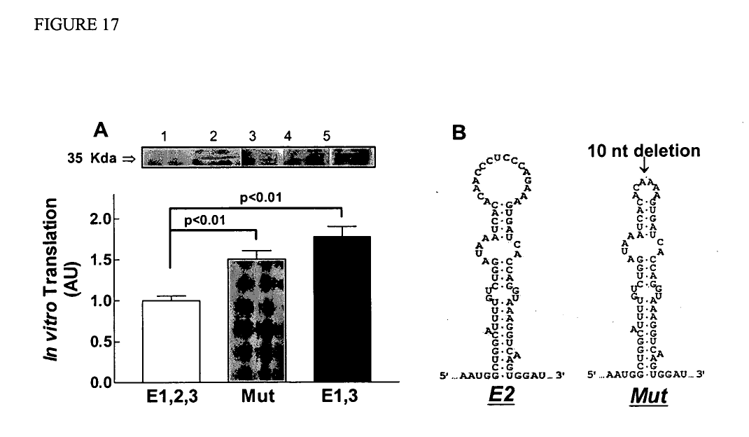

Site directed mutagenesis: Ten nucleotides within the loop of the E2 hairpin predicted by the Zuker algorithm (Zuker, 2000), were deleted by site-directed mutagenesis using the Quick Change site-directed mutagenesis system (Stratagene) (see FIG. 17).

In vitro translation: E1,3, E1,2,3 and mutant E1,2,3 cDNAs were subcloned into the pcDNA5/FRT expression vector (Invitrogen). These plasmid DNAs were linearized by Xho I digestion. After confirming that the digestion was complete on an agarose gel, linearized DNA was purified by the PCR purification system (Qiagen), then in vitro transcribed into capped RNA (T7 mMessage mMachine, Ambion). Transcribed RNAs were quantified using the Ribogreen RNA quantitation method (Molecular Probes). RNA (1.5 μg) was translated in wheat germ extracts in the presence of [35S]-methionine (Promega), as described (Ji et al., 2000). Dose response curves of E1,3 and E1,2,3 RNA revealed that 1.5 μg yielded maximum levels of AT1R protein under the experimental conditions. The translated protein was analyzed by SDS-gel electrophoresis. Protein size was determined by comparison with broad-range prestained SDS-PAGE markers. Autoradiograms of SDS gels were quantified by phosphorimaging and expressed in arbitrary units.

Northern analysis: Total RNA (20 □g) from Sprague Dawley rat adrenals was isolated by Tri Reagent (Molecular Research Center, Cincinnati, Ohio) and then subjected to formaldehyde gel electrophoresis (1%) followed by transfer of the denatured RNA to positively charged nylon membranes (Micron Separations, Westborough, Mass.) as described in the manufacturer's protocol. The membrane was prehybridized in hybridization buffer (5×SSC, 5× Denhardt's, 0.5% SDS, 100 μg/ml salmon sperm DNA) at 65° C. for 3 h. Hybridization was performed with [32P]-dCTP labeled probes generated by PCR (E1: 124 bp, nucleotide position, −271 to −147; E2: 87 bp, nucleotide position, −140 to −53; and, E3: 485 bp, nucleotide position, −1492 to −1978) at 65 ° C. for 20 h. After hybridization, the membranes were washed successively in: 2×SSC, 0.1% SDS at room temperature for 15 min; 1×SSC, 0.1% SDS at 42° C. for 30 min; and, 0.1×SSC, 0.1% SDS at 42° C. for 30 min. The membranes were then exposed to Kodak Biomax MS film for 5 days at −80° C.

Real-time PCR: Total RNA was extracted using TRIzol reagent (Life Technologies). First strand cDNA was made from total RNA using iScript cDNA synthesis kit (BioRad) with MMLV RNase H+ reverse transcriptase, oligo(dT) and random hexamers. Quantitations of specific mRNAs and 18S rRNA (for control) were performed by real-time PCR using the ABI Prism 7700 Sequence Detection System (Perkin Elmer Applied Biosystems). The PCR reaction mixture consisted of RNase free water, TaqMan Universal PCR Master Mix (Perkin Elmer Applied Biosystems) and 300 nM specific primers and 10 μM probe (Forward primers: 119F (E1,2,3), 5′-CCA CAT TCC CTG AGT TAA CAT ATG A-3′(SEQ ID NO: 7) and 114F (E1,3),5′-CTC TGC CAC ATT CCC TGG TC-3′(SEQ ID NO: 8); Reverse primer: 310R (E1,2,3 & E1,3), 5′-TCT TTT GAT ACC ATC TTC AGC AGA A-3′) (SEQ ID NO: 9); and Probe: 232T (E1,2,3 & E1,3), 6 FAM-TCG AAT AGT GTC TGA GAC CAA CTC AAC CCA-TAMRA) (SEQ ID NO: 10), and cDNA samples. PCR conditions were optimized for the probe (232T) and both sets of primers (119F & 310R and 114F & 310R) using control cDNAs. The specificity of these primers were confirmed in CHO cells stably expressing E1,3 or E1,2,3. That is, Applicants did not detect any amplified products using E1,3 specific primers in the E1,2,3 expressing cells and vice versa. The expression of 18S rRNA, E1,3 and E1,2,3 mRNA in each sample was quantitated using respective primers. PCR reactions without reverse transcription were included to control for contamination by genomic DNA. The standard curves for 18S rRNA, E1,3 and E1,2,3 mRNA were made from a series of ten times dilutions (53, 54, 55, 56, 57, and 58) for each cDNA. The cell and tissue levels of these cDNAs were calculated based on the standard curves.

Statistics: Data were expressed as means±SEM. Statistical significance of the differences between groups were assessed by Student's t-test. Statistical differences between time courses were assessed by one-way ANOVA. Differences were considered significant at p<0.05

Results

The membrane density of AT1aRs is markedly higher in CHO cells stably expressing the E1,3 transcript compared to the E1,2,3 variant.

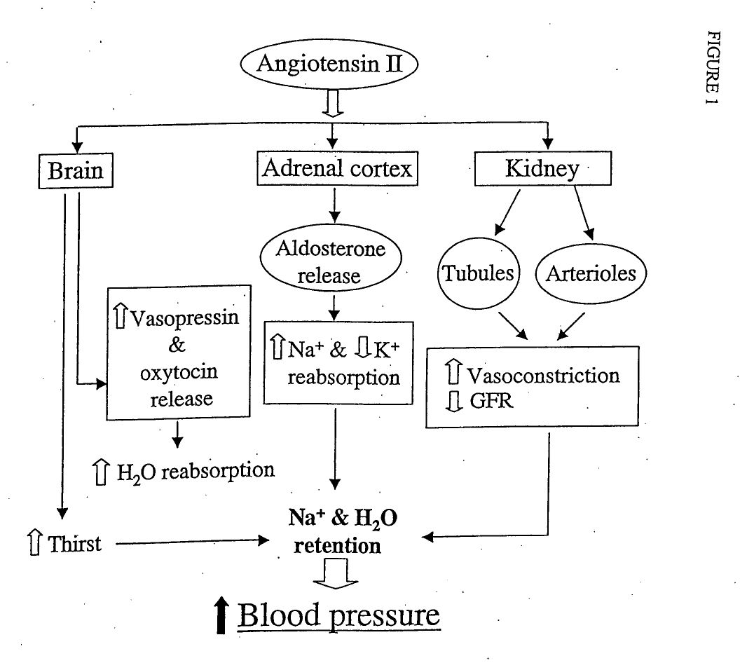

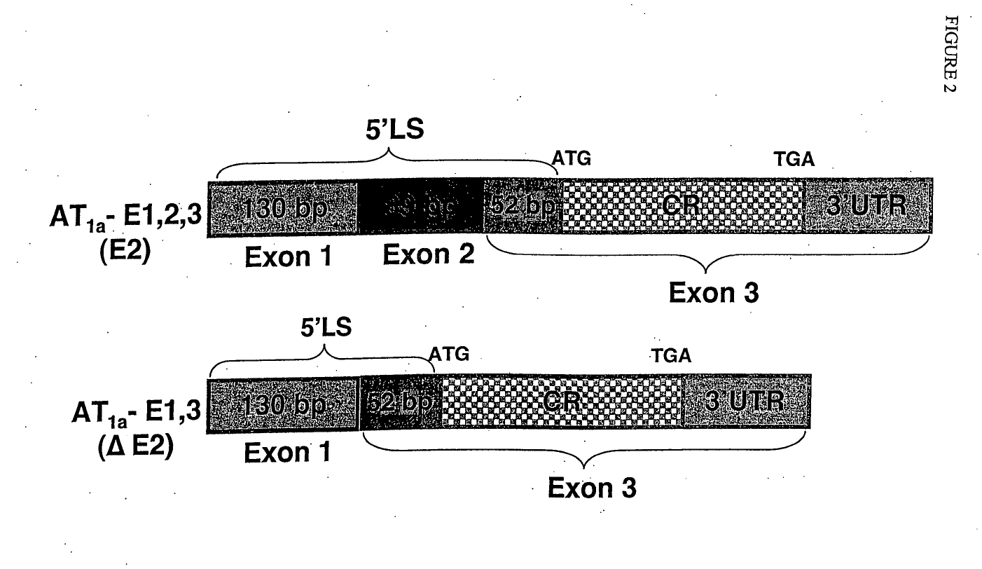

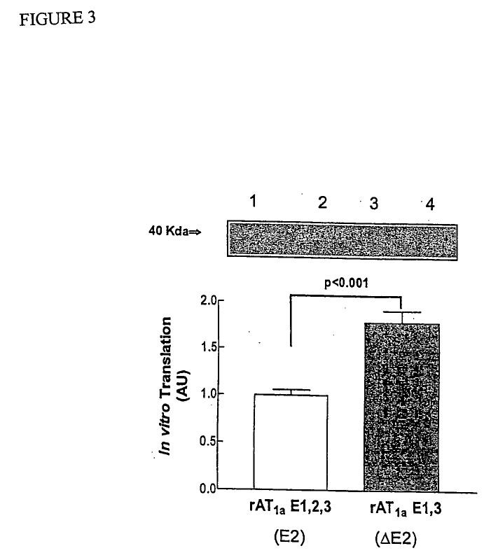

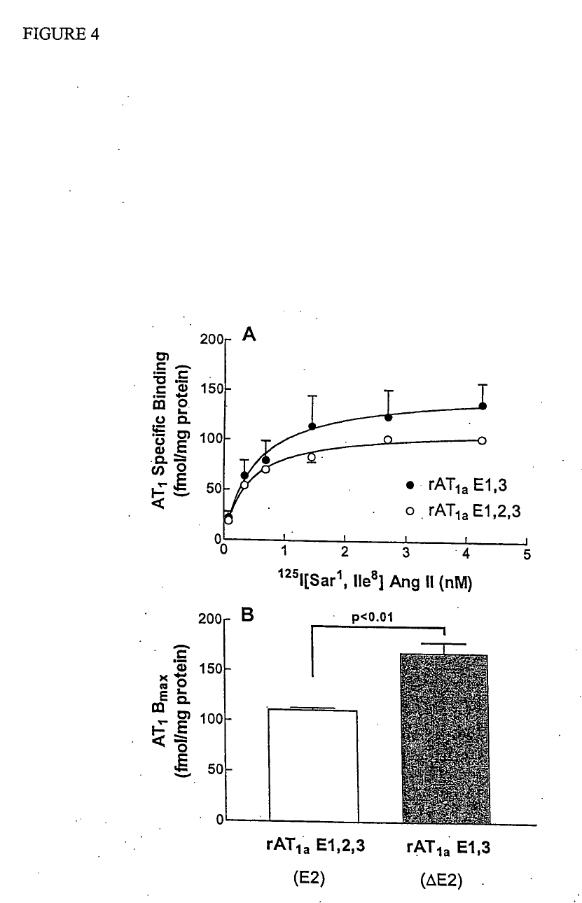

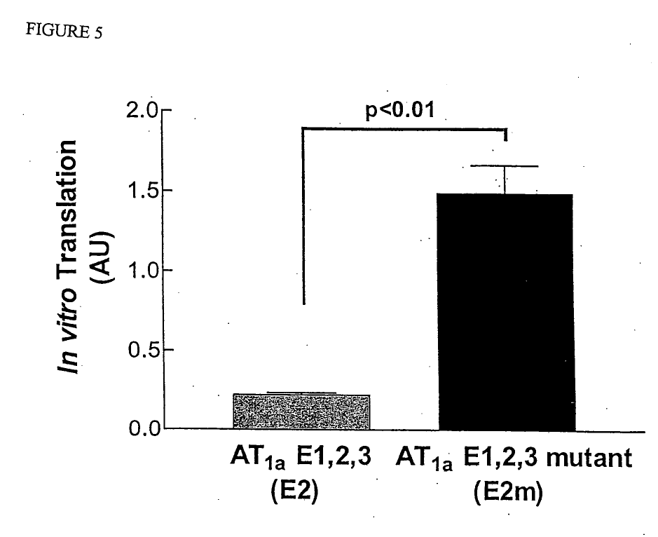

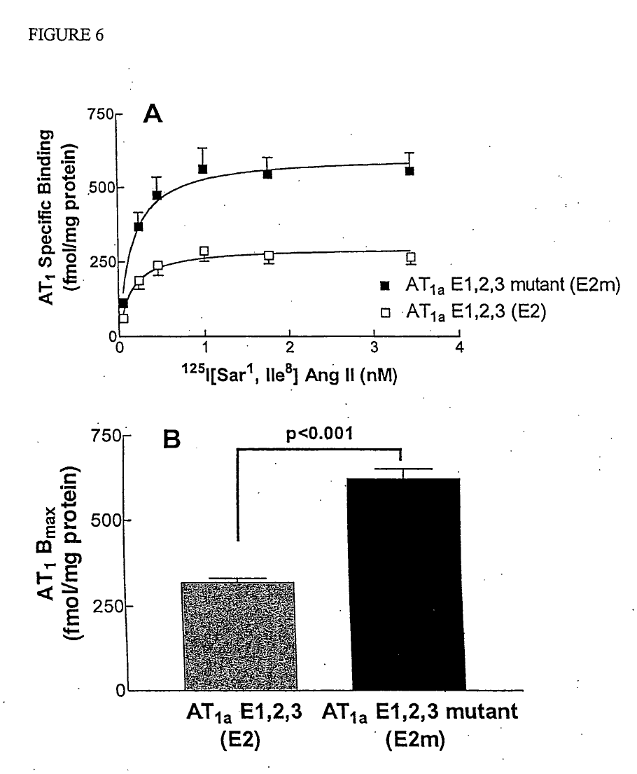

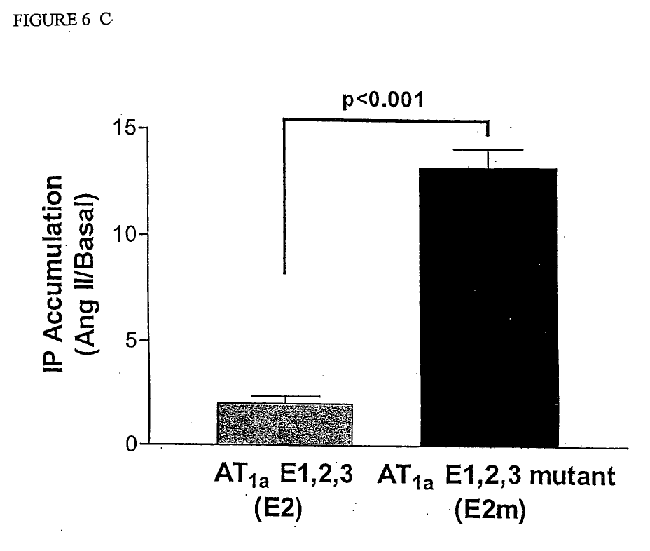

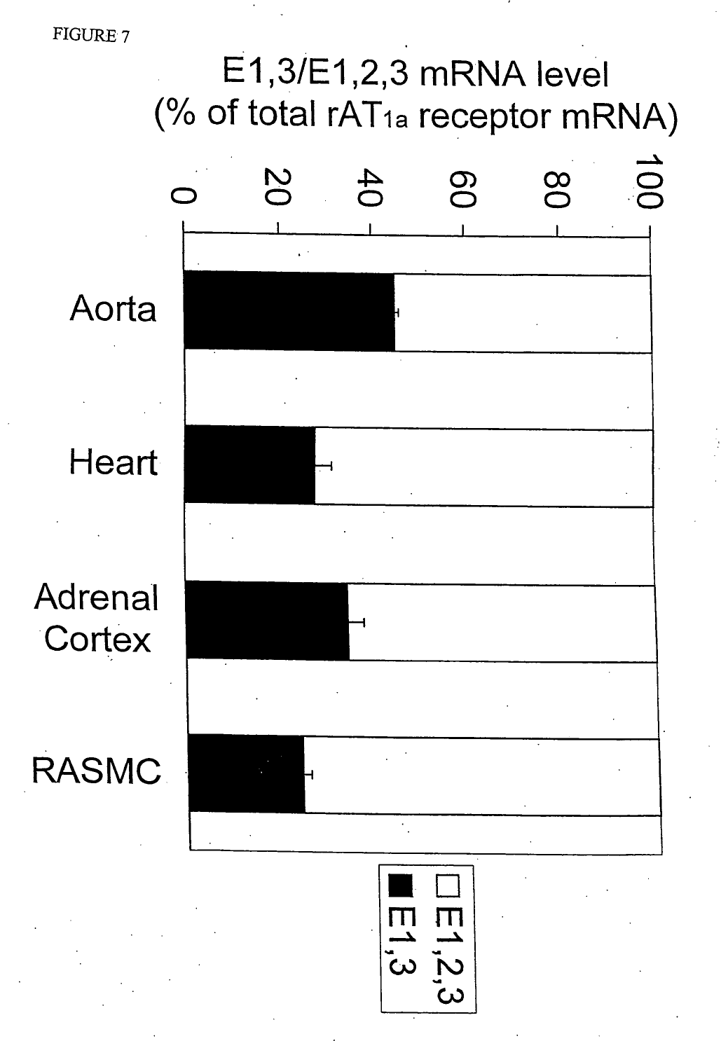

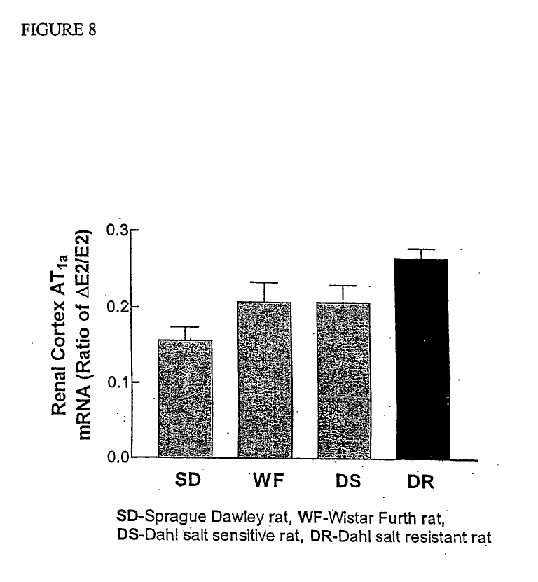

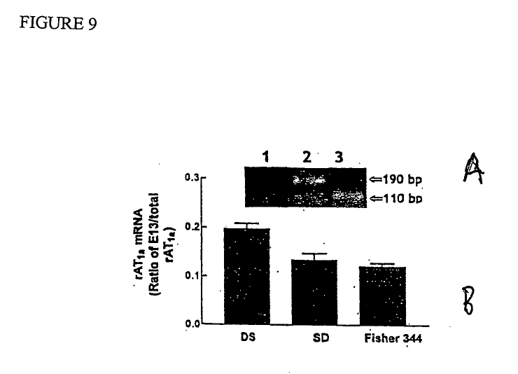

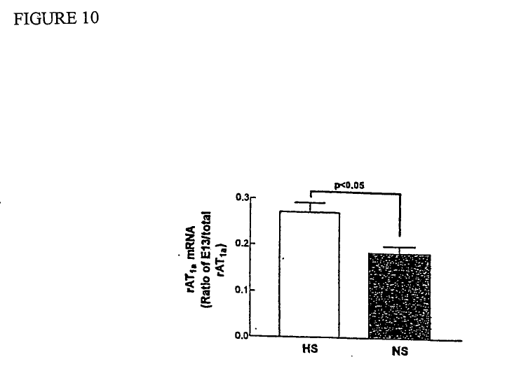

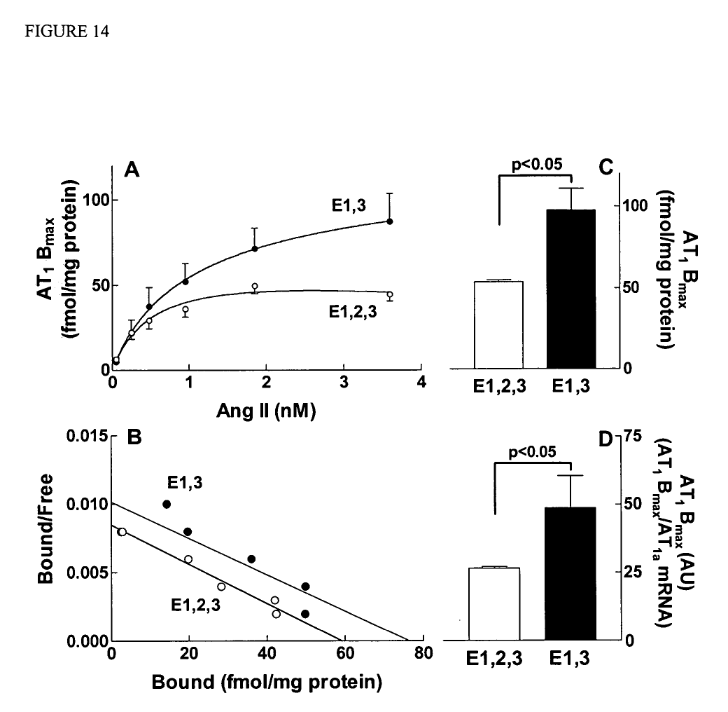

Saturation isotherms using 125I[Sar1,Ile8]Ang II were performed on membranes isolated from stably transfected CHO cells expressing the E1,3 or the E1,2,3 transcript (FIG. 14A). Scatchard analysis (FIG. 14B) revealed that AT1R density (Bmax) was 1.8-fold higher in CHO cells stably expressing the E1,3 transcript compared to the E1,2,3 variant (FIG. 14C) and 1.8-fold higher when normalized to AT1R mRNA levels (FIG. 14D); no significant differences in receptor affinity (Kd) were observed (E1,3, 0.73±0.4 nM vs E1,2,3, 0.39±0.10 nM, n=3).

Ang II-stimulated IP production is markedly higher in CHO cells stably expressing the E1,3 transcript compared to the E1,2,3 variant.

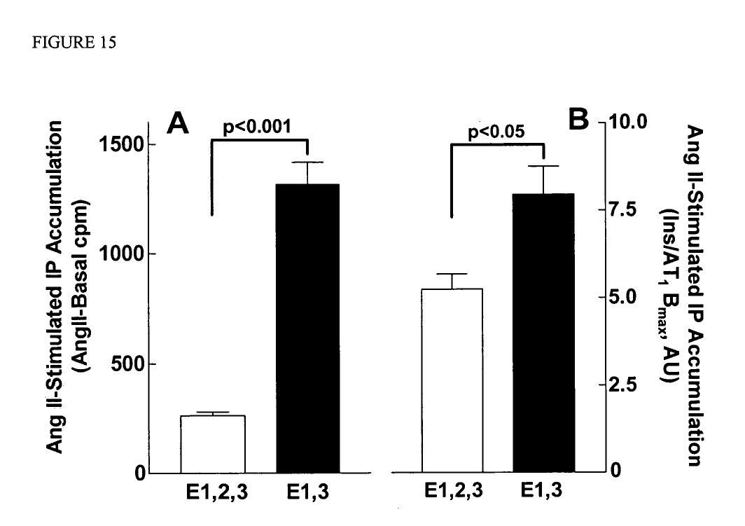

Stably transfected CHO cells expressing the E1,3 or the E1,2,3 transcript were incubated with [3H]inositol for 16 hours before incubation with Ang II. CHO cells expressing the E1,3 transcript accumulated 5-fold higher levels of IP compared to the E1,2,3 transfected cells after stimulation with 100 nM Ang II for 20 min (FIG. 15A) and 1.5-fold higher levels when normalized to AT1R Bmax (FIG. 15B).

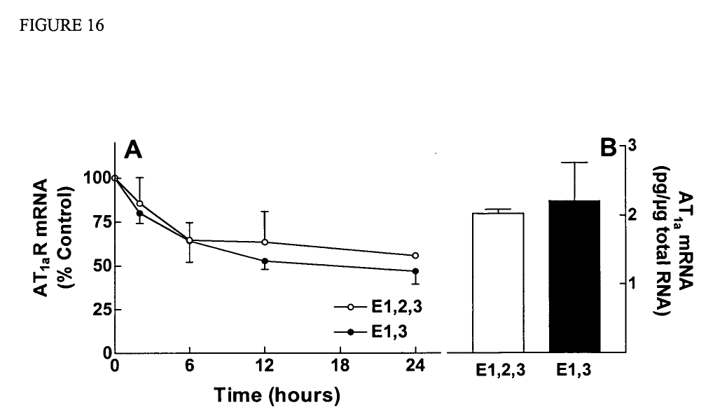

No differences in E1,3 and E1,2,3 mRNA levels or mRNA turnover were found To assess mRNA turnover, stably transfected CHO cells expressing the E1,3 or the E1,2,3 transcript were treated with the transcription inhibitor, actinomycin D (20 μg/ml) for 24 hours before cells were collected at 0, 2, 6, 12 and 24 hour time points. No differences in AT1aR mRNA stability were observed between cells expressing E1,3 and those expressing E1,2,3 transcripts (FIG. 16A). Moreover, no differences in AT1R mRNA levels were observed in CHO cells stably expressing E1,3 and E1,2,3 (FIG. 16B).

E1,3 and E1,2,3 plasmid DNAs were linearized then in vitro transcribed into capped RNA. RNA (1.5 μg) was translated in wheat germ extracts in the presence of [35S]methionine and analyzed by phosphorimaging of autoradiograms of SDS gels (FIG. 17, Upper panel). These in vitro translation studies revealed that 1.8-fold more AT1R protein (40 KDa) was synthesized by the E1,3 transcript compared to the E1,2,3 variant (FIG. 17, left panel). Based on a stable hairpin within E2, which was predicted by the Zuker algorithm (Zuker, 2000), Applicants deleted 10 nucleotides that comprised the loop by site-directed mutagenesis (FIG. 17, right panel). Deletion of this loop at the top of the hairpin increased in vitro translation to levels that were indistinguishable from E1,3 (FIG. 17, left panel).

All Three AT1aR Exons are Expressed in Rat Tissue

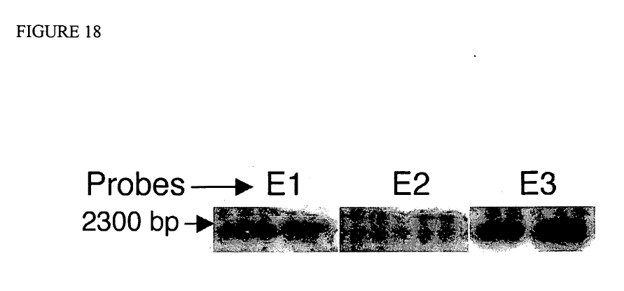

Total RNA was isolated from the adrenal cortex from 4 Sprague Dawley rats for Northern blot analysis using E1, E2 and E3 specific probes. All three probes hybridized to a 2.2 kb band, indicating that all three exons are expressed in this tissue (FIG. 18).

Tissue Specific Regulation of Alternative Splicing

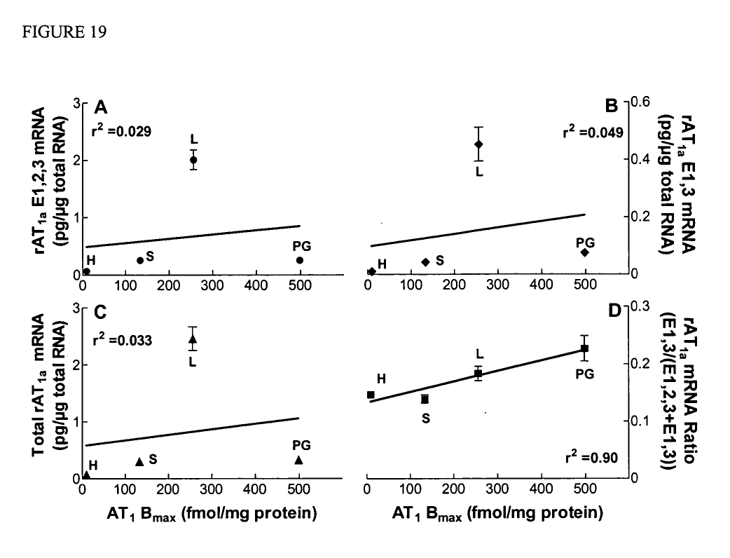

The AT1aR splice variant composition and AT1R densities were determined in rat heart, spleen, liver, and preglomerular tissues. These tissues were chosen because the levels of AT1bR mRNA were less than 20% of the total AT1R (AT1aR+AT1bR) mRNA population (data not shown). In all four tissues, E1,2,3 was the predominant splice variant; however, tissue specific differences existed in the ratio of the E1,3 and E1,2,3 splice variants. No correlation was found between AT1R densities and the mRNA levels of E1,2,3 (r2<0.03, FIG. 19A), E1,3 (r2<0.05, FIG. 19B), total AT1aR (r2<0.04, FIG. 19C) or total AT1aR+AT1bR (r2<0.05, data not shown). In contrast, there was an excellent correlation between AT1R densities and the percentage of E1,3 in the AT1aR mRNA population (r2=0.90, FIG. 19D).

Discussion

This study suggests that E2 within the 5′LS of the AT1aR harbors a translational inhibitory element that results in reduced AT1aR expression and signaling. Results show that CHO cells stably expressing the E1,3 transcript expressed significantly higher levels of AT1Rs in their membranes compared to the cells expressing the E1,2,3 variant. This difference in receptor expression between these two variants correlated with the differences in Ang II-induced inositol phosphate signaling; the cells expressing the E1,3 transcript produced significantly more inositol phosphates in response to Ang II stimulation than the cells expressing the E1,2,3 variant. Not only do these data indicate that E2 inhibits receptor expression, these findings support previous studies in vascular smooth muscle cells that show a tight correlation between AT1R number and the magnitude of AT1R signal transduction (Lassegue et al., 1995). Therefore, changes in AT1R number due to splice variant expression could have profound physiological effects due to changes in the magnitude of AT1R signaling. That is, even though the E1,3 and E1,2,3 variants code for identical AT1aR proteins, different ratios of E1,3 to E1,2,3 mRNA levels could alter AT1aR activity.

The finding that no differences were observed in mRNA turnover or mRNA levels suggests the increased receptor expression in cells expressing the E1,3 variant is not due to increased mRNA stability. The data showing that the E1,3 transcript is translated in vitro with greater efficiency than the E1,2,3 variant suggests that E2 is inhibitory to translation and that this inhibition of translation contributes to the differences in AT1R expression in cells. Applicants results are similar to a recent report demonstrating that two 5′LS splice variants of neuronal nitric oxide synthase exhibit markedly different translational efficiencies even though they code for identical proteins (Newton et al., 2003).

Studies in transiently transfected cells in which the 5′LS was subcloned in front of a reporter gene suggests that the human AT1 and AT2 receptors contain an inhibitory exon in the 5′LS (Curnow et al., 1995; Warnecke et al., 1999). Findings presented herein extend these previous reports by showing that exon 2 in the 5′LS of the rat AT1aR actually decreases AT1R number and signaling capability in cells stably expressing the full length transcripts. This is an important point because RNA elements taken out of context may behave differently due to differences in RNA folding or to interactions from downstream elements. In addition, they show that reduced receptor number is likely the result of decreased mRNA translation rather than reduced mRNA stability.

Applicants data showing that in vitro translation was markedly increased after deletion of 10 nucleotides in a loop within a predicted hairpin in E2 suggests that secondary structures such as stem loops could influence protein expression by serving as recognition sites for RNA binding proteins that inhibit translation (Krishnamurthi et al., 1999; Wu et al., 2003). Internal ribosome entry (IRE) sites could also influence the efficiency of translation. Martin et al. (Martin et al., 2003) has recently found that translation of the human AT1R is mediated by a highly efficient IRE within E1. Thus, alternative splicing in the 5′LS provides a mechanism for introducing or removing regulatory elements in both the rat and human AT1R that control receptor expression post-transcriptionally.

The finding presented herein that the percentage of E1,3 in the total AT1aR mRNA population varies in different rat tissues indicates that alternative splicing of the AT1aR is regulated in a tissue-specific manner. Furthermore, the tight correlation between the percentage of E1,3 and AT1R densities strongly suggests that tissue-specific regulation of alternative splicing contributes to tissue specific differences in AT1R densities. The Northern blot showing that all three exons are expressed in rat tissue not only confirms our real-time PCR data indicating the presence of E1,2,3 but also demonstrates that the PCR data is not a cDNA artifact of incomplete splicing. This point is further made by the fact that E1,2,3 is the predominant splice variant in the AT1aR mRNA population. The possibility that dysregulation of alternative splicing could contribute to pathophysiology is raised by a previous report showing that the exon composition of the human AT1R in the atria and left ventricle were altered by heart failure (Warnecke et al., 1999).

The observation that no correlation exists between AT1R density and the E1,3, E1,2,3 or total AT1R mRNA levels in rat tissues supports previous studies demonstrating that the AT I aR is post-transcriptionally regulated (Wu et al., 2003). This lack of correlation is also consistent with post-translational regulatory mechanisms such as receptor desensitization, internalization and recycling (Thomas, 1999). Much evidence indicates that the cell employs multiple regulatory mechanisms at the transcriptional, post-transcriptional and post-translational levels so that tight control over the expression and function of the AT1R is obtained. AT1R regulation is further complicated in the rat and mouse because of the presence of two subtypes of the AT1 receptor and because 5′LS splice variants of the AT1bR are also likely to exist.

In summary, these data indicate that even though the E1,3 and E1,2,3 transcripts code for identical proteins, stable expression of these transcripts results in marked differences in AT1R densities and receptor signaling. The differences in AT1R densities and activity are likely the result of differences in translational efficiency rather than differences in mRNA stability. Furthermore, the data suggests that the loop in a predicted hairpin in E2 contributes to the RNA inhibitory cis element within this exon. Lastly, the data indicate that regulation of AT1R splicing is tissue-specific and may contribute to tissue-specific differences in AT1R densities. Elucidating the molecular mechanisms regulating AT1R alternative splicing will facilitate endeavors to define the genetic, physiological, cellular and biochemical mechanisms that underlie changes in the renin angiotensin system that could contribute to the pathology of hypertension and associated cardiovascular and renal disease.

### REFERENCES

Curnow, K. M., et. al. Mol. Endocrinol. 9 (1995) 1250-1262.
Elton, T. S. and Martin, M. M.: Trends Endocrinol Metab 14 (2003) 66-71.
Ji, H., et. al. Meth Mol Med 51 (2000) 171-192.
Ji, H., et. al. J. Biol. Chem. 269 (1994) 16533-16536.
Krishnamurthi, K., et. al. Endocrinology 140 (1999) 5431-5434.
Lander, E. S., et. al. Nature 409 (2001) 860-921.
Lassegue, B., et. al. Mol. Pharmacol. 48 (1995) 601-609.
Llorens-Cortes, C., et. al. Hypertension 24 (1994) 538-48.
Martin, M. M., et. al. Mol Cell Endocrinol 212 (2003) 51-61.
Newton, D. C., et. al. Journal of Biological Chemistry 278 (2003) 636-44.
Nickenig, G. and Murphy, T. J.: Mol. Pharmacol. 50 (1996) 743-751.
Ouali, R., Berthelon, M.-C. and Begeot, M.: Endocrinology 138 (1997) 725-733.
Owonikoko, T. K., et. al. J Nucl Med 45 (2004) 94-100.
Smith, C. W. and Valcarcel, J.: Trends in Biochemical Sciences 25 (2000) 381-8.
Thomas, W. G.: Regul Pept 79 (1999) 9-23.
Tian, Y. a. K. S.: J Tiss. Cult. Meth. 15 (1993) 87-92.
Vanderheyden, P. M., et. al. British Journal of Pharmacology 126 (1999) 1057-65.
Warnecke, C., et. al. J Mol Med 77 (1999) 718-27.
Wu, Z., et. al. Endocrinology 144 (2003) 3251-61.
Zheng, W., et. al. Kidney Int 59 (2001) 1881-90.
Zuker, M.: Curr Opin Struct Biol 10 (2000) 303-10.

### Example 13

### Assessment of Translation Efficiency of E1,3 Splice Variant in Cells that Endogenously Express Both AT1a Receptor Splice Variants

The differences in the composition of the 5′UTR of E1,3 and E 1,2,3 rat AT1a receptor mRNAs suggest that the two transcripts may be differentially regulated. Applicants have found that the E1,3 splice-variant is translated more efficiently than E1,2,3 mRNA in transfected cells and in vitro translation assays (Example 12). Described in this example are assessment of whether the E1,3 splice variant is also translated more efficiently in cells that endogenously express both AT1a receptor splice variants and the resulting findings. Furthermore, Applicants previous finding that the E1,2,3 splice variant is the predominant subtype led them to ask a second question: given that E1,3 mRNA is less abundant but more efficiently translated than the E1,2,3 mRNA, what is its relative contribution to synthesis of the AT1a receptor? To answer these two questions, Applicants used splice variant-specific small interfering RNA (siRNA)-mediated RNA interference (RNAi) (Caplen et al., 2001; Elbashir et al., 2002) to selectively silence the E1,3 splice variant in rat aortic smooth muscle cells (RASMC) while leaving the E1,2,3 splice variant intact. They then measured the effect of this siRNA treatment on functional AT1 receptor protein by measuring AT1 receptor radioligand binding.

Materials and Methods

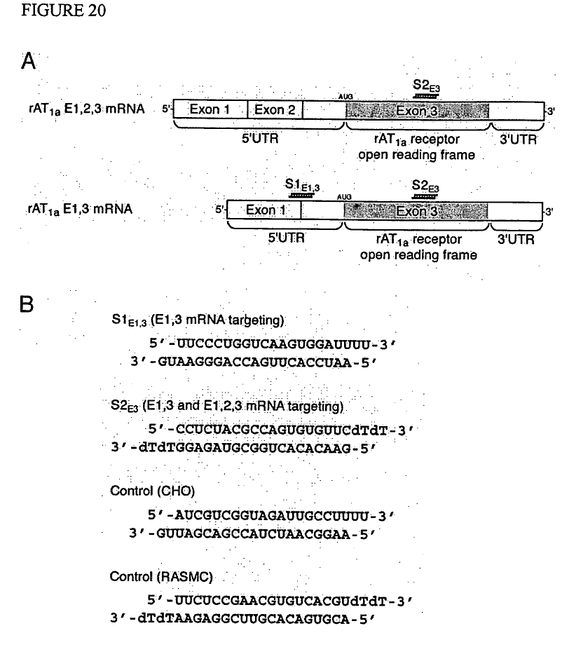

Small Interfering RNA (siRNA) Constructs. RNAi was induced by transfection of cells with chemically synthesized siRNA. To selectively silence the E1,3 splice variant, an siRNA duplex (S1E1,3) was selected which targeted the junction between exons 1 and 3. A second siRNA (S2E3), which targeted a sequence in exon 3 was selected for use as a postive control (FIG. 20A). BLAST searching showed that neither siRNA selected sequence had any significant matches to any other known mammalian genes. Two siRNA sequences were selected for use as non-silencing controls in RASMC and Chinese Hamster Ovary cells. All siRNA sequences are listed in FIG. 20B. All siRNAs were chemically synthesized by Qiagen Inc. (Valencia, Calif.). Each siRNA was dissolved in suspension buffer (100 mM KOAc, 30 mM HEPES KOH and 2 mM MgOAc, pH 7.4) at a concentration of 20 μM and stored in aliquots at −20° C. until use. siRNA Transfections. Chinese Hamster Ovary (CHO) cells stably expressing either E1,3 (CHO-E1,3) or E1,2,3 (CHO-E1,2,3) AT1a receptor mRNA were generated and cultured as previously described (Example 12). For transfection, cells were sub-cultured with Trypsin[Versene and plated at a density of either 2.5×104 cells/cm2 (CHO-E1,3) or 5×104 cells/cm2 (CHO-E1,2,3) in Dulbecco's Modified Eagle's Medium (DMEM) supplemented with 10% fetal bovine serum (FBS) and 2 mM 1-glutamine. 24 h after plating, the medium was replaced and the cells were transfected with siRNA complexed with the cationic liposomal transfection reagent, lipofectamine 2000 (Invitrogen, Carlsbad Calif.), according to the Invitrogen protocol. Immediately before complexing with lipofectamine, the siRNA was annealed by heating for 1 min at 90° C. and then incubated for 1 h incubation at 37° C. The final siRNA and lipofectamine 2000 concentrations were 100 nM and 1,667 ng/μl, respectively.

RASMC (Cell Applications, San Diego, Calif.) from male Sprague Dawley rats were cultured in DMEM/F-12 supplemented with 10% FBS and 2 mM 1-glutamine. For transfection, cells were sub-cultured with Trypsin/Versene and plated at a density of 1×104 cells/cm2 in DMEM supplemented with 10% FBS and 2 mM 1-glutamine. 48 h after plating, the medium was replaced and the cells were transfected with siRNA/lipofectamine 2000 as described above. The final siRNA and lipofectamine 2000 concentrations were 200 nM and 3,333 ng/μl, respectively. Real-time PCR. Total RNA was extracted from RASMC using Trizol reagent (Molecular Research Center, Cincinnati, Ohio). The total RNA was quantitated by the RiboGreen RNA Quantitation system (Molecular Probes, Eugene, Oreg.). First strand cDNA was prepared from total RNA using the iScript cDNA Synthesis method (BioRad) with random hexamer primers. Quantitation of AT1a receptor splice variant mRNA and 18S rRNA (for control) were performed by real-time PCR using the ABI Prism 7700 Sequence Detection System (Applied Biosystems Inc., Foster City, Calif.). The AT1a receptor splice variant-specific primers (300 nM) sequences were as follows: Forward primers: 119F (AT1a receptor E1,2,3), 5′-CCA CAT TCC CTG AGT TAA CAT ATG A-3′(SEQ ID NO: 11) and 114F (AT1a receptor E1,3), 5′-CTC TGC CAC ATT CCC TGG TC-3′ (SEQ ID NO: 12); Reverse primer: 310R (for both AT1a receptor E1,2,3 & AT1a receptor E1,3), 5′-TCT TTT GAT ACC ATC TTC AGC AGA A-3′) (SEQ ID NO: 13); and Probe (10 μM): 232T (AT1a receptor E1,2,3 & AT1a receptor E1,3), 6 FAM-TCG AAT AGT GTC TGA GAC CAA CTC AAC CCA-TAMRA) (SEQ ID NO: 14). PCR conditions were optimized for the probe (232T) and both sets of primers (119F & 310R and 114F & 310R) using control cDNAs. PCR reactions without reverse transcription were included to control for contamination by genomic DNA. The standard curves for 18S rRNA and AT1a receptor mRNA were made by a series of five-times dilutions (53, 54, 55, 56, 57, and 58) of the cDNA. The standard curves were calculated based on the control values.

AT1 receptor radioligand binding assay. Whole cell AT1 receptor binding was measured as previously described (Ji et al., 1995). Briefly, 48 h after transfection, the cell medium was aspirated and replaced with monoiodinated 125I[Sar1Ile8]Ang II (2-3×105 cpm; Peptide Radioiodination Service, Pullman, Wash.) in DMEM/F-12+0.1% BSA. After incubation at room temperature for 90 min, unbound ligand was removed by washing each well twice with 1 ml ice-cold phosphate buffered saline (PBS). Bound ligand was recovered by dissolving the protein in each well with 1 ml 0.01 M NaOH. The quantity of 125I[Sar1Ile8]Ang II present in each sample was determined using a Cobra γ-spectrophotometer (Packard Bell, Palo Alto, Calif.).

Statistical Analysis. All data are reported as the mean±SEM. When comparisons were made between two different groups, statistical significance was determined using Student's t-test. When multiple comparisons were made, statistical significance was determined using one-way ANOVA followed by Newman-Keuls' post-test.

Results

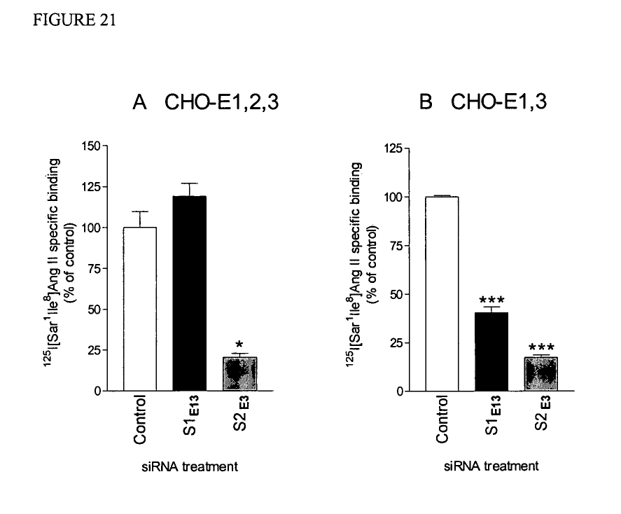

Specificity of AT1a Receptor Splice Variant-Targeting siRNA Designs. To evaluate the specificity of the siRNA constructs, CHO cells stably expressing either the E1,3 or E1,2,3 splice variant of the rat AT1a receptor mRNA were transfected with the siRNA constructs targeting either the E1,3 variant (S1E 1,3), the coding region of the receptor (S2E3) or with non-silencing control siRNA. 48 h after transfection, 125I[Sar1Ile8]Ang II binding to the cells was measured. Transfection of CHO cells expressing the E1,3 splice variant (CHO-E1,3) S1E1,3 siRNA induced a 59.5±2.9% (n=3, p<0.001 [Newman-Keuls]) reduction in AT1 receptor binding compared to the control siRNA. In contrast, S1E1,3 siRNA treatment had no effect on AT1 receptor binding in cells expressing the E1,2,3 splice variant (CHO-E1,2,3)(n=4, NS). Transfection with S2E3 siRNA caused marked reductions in binding compared with control in both CHO-E1,3 (82.8±1.3%; n=3, p<0.001) and CHO-E1,2,3 (79.3±2.3%; n=4, p<0.001) cells (FIG. 21).

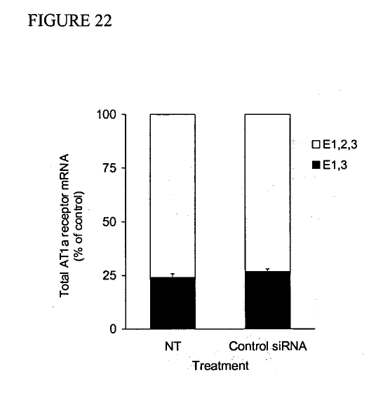

E1,2,3 mRNA is the Predominant AT1a Receptor Splice Variant in RASMC. Measurement of E1,2,3 and E1,3 mRNA levels by quantitative real-time PCR in RASMC not treated (NT) with siRNA revealed that the major AT1a receptor splice variant expressed in RASMC is E1,2,3; E1,2,3 mRNA accounted for 75.9±1.9% (n=4) of the total AT1a receptor mRNA population, while the E1,3 transcript accounted for the remaining 24.1 % of the total AT1a receptor mRNA (FIG. 22). Treatment with non-silencing control siRNA had no effect on the relative proportions of E1,3 and E1,2,3 mRNA in RASMC; following 48 h treatment with control siRNA, the E1,2,3 transcript remained at 73.2±1.2% of the total AT1a receptor mRNA population (FIG. 22).

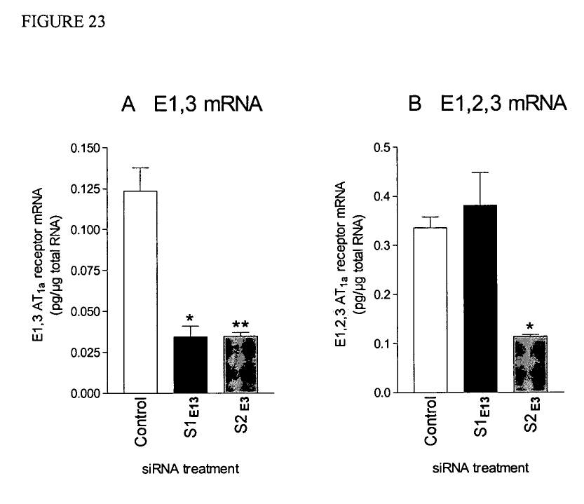

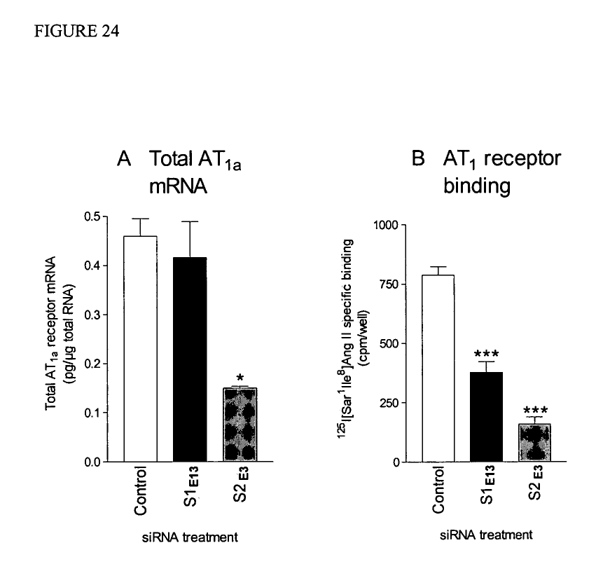

AT1a Receptor Splice Variant-Specific RNAi in RASMC. 48 h after siRNA treatment, RNA was extracted from cells and assayed by quantitative real-time RT-PCR. FIG. 23A shows the effect of siRNA treatment on E1,3 mRNA levels. Treatment with S1E1,3 siRNA induced a 72.1±5.3% reduction in E1,3 mRNA (n=2, p<0.05). Similarly, treatment with S2E3 siRNA resulted in a 71.7±1.6% reduction in E1,3 mRNA (n=2, p<0.01). In contrast, treatment with S1E1,3 siRNA had no effect on E1,2,3 mRNA, while treatment with S2E3 siRNA caused a 65.7±0.6% reduction in E1,2,3 mRNA (n=2, p<0.05, FIG. 23B). Although S1E1,3 siRNA treatment induced a marked reduction in E1,3 mRNA in RASMC (FIG. 23), this reduction amounted to only a small reduction in total AT1a receptor mRNA, since the E1,3 splice variant accounts for only a small proportion of total AT1a receptor mRNA. Indeed, when Applicants calculated total AT1a receptor mRNA by adding E1,3 and E1,2,3 mRNA levels together, Applicants were unable to detect any significant reduction in total AT1a receptor mRNA following S1E1,3 siRNA treatment compared with the control (FIG. 24A). In contrast, treatment with S2E3 siRNA caused a reduction in the total AT1a receptor mRNA of 67.3±0.9% (FIG. 24A).

Disproportionate Inhibition of AT1 Receptor Binding by E1,3 Splice Variant-specific RNAi in RASMC. In order to assess the effects of splice variant-specific siRNA on functional AT1 receptor protein expression, Applicants measured AT1 receptor binding after siRNA treatment. Treatment with both S1E1,3 and S2E3 siRNA induced large reductions in AT1 receptor binding of 52±5.8% (n=4, p<0.001) and 79.8±3.6% (n=4, p<0.001), resp compared to treatment with control siRNA (FIG. 23B). While treatment with both S1E1,3 and S2E3 siRNA induced reductions in AT1a receptor mRNA, the inhibitory effect of S1E1,3 siRNA treatment on AT1 receptor binding was disproportionately large (FIG. 23B) given its minimal effect on the total AT1a receptor mRNA level (FIG. 23A). Data are summarized in Table 1. Note that these cells have undetectable mRNA levels of the AT1b receptor subtype and therefore all AT1 receptor protein arises from the AT1 a subtype (data not shown).

TABLE 1
Table 1. Selective knockdown of AT1a receptor mRNA induces a
disproportionately large reduction in binding relative to the reduction
in total AT1a receptor mRNA. When compared using Student's t-test,
the reduction in binding induced by S1E1,3 siRNA is revealed
to be significantly greater than its effect on AT1a receptor mRNA.
In contrast, there was no significant difference between the
effect of S2E3 siRNA on total AT1a receptor mRNA and AT1
receptor binding.

| | | AT₁ receptor | |
|---|---|---|---|
| | Total AT₁ₐ receptor | binding | |
| Treatment | mRNA (% of control) | (% of control) | P (Student's t-test) |
| S1_{E1,3} | 90.6 ± 15.9 | 47.9 ± 5.8 | p < 0.05 |
| S2_{E3} | 32.7 ± 0.9 | 20.2 ± 3.6 | NS |

Discussion

The aim of this study was to use selective silencing of rat AT1a receptor splice variants by RNAi to investigate the role splicing plays in regulation of receptor expression. The first step was to design siRNA sequences which could selectively mediate the degradation of the E1,3 transcript (i.e. S1E1,3) or both the E1,3 and E1,2,3 transcripts (S2E3). Testing of these siRNA designs in CHO cells expressing either the E1,3 or E1,2,3 AT1a receptor splice variants demonstrated that the chosen siRNA designs induced selective degradation of their intended targets in this cell type.

Given these results, Applicants proceeded to use these siRNA constructs to investigate the role of splicing in the regulation of AT1a receptor expression in RASMC, a primary cell line that endogenously expresses both AT1a receptor splice variants. Analysis of AT1a receptor mRNA levels following S1E1,3 and S2E3 siRNA treatment of RASMC confirmed that the siRNA designs mediated highly specific degradation of their intended targets in cells that endogenously express both transcripts. It is important to note that the E1,2,3 splice variant is predominant in RASMC, making up 76% of the total AT1a receptor mRNA pool in these cells, with E1,3 mRNA accounting for the remainder (FIG. 22). Thus, although S1E1,3 siRNA treatment induced a marked and significant reduction in E1,3 mRNA, this reduction amounted to only a small reduction in total AT1a receptor mRNA. Indeed, when Applicants calculated total AT1a receptor mRNA by adding E1,3 and E1,2,3 mRNA levels together, Applicants were unable to detect any significant reduction in total AT1a receptor mRNA following S1E1,3 treatment compared with the control. However, treatment with S1E1,3 induced a marked reduction in AT1 receptor binding 48 h after transfection. This effect on binding was disproportionately large given the marginal effect of this treatment on levels of AT1a receptor mRNA. In contrast, treatment with S2E3 siRNA caused a reduction in total AT1a receptor mRNA of 67.3±0.9% (FIG. 24A), and a reduction in AT1 receptor binding of equivalent magnitude.

Taken together, these results suggest that E1,3 mRNA is considerably more efficiently translated than E1,2,3 mRNA in cells in which both splice variants are endogenously expressed. Thus, these findings extend Applicants' previous in vitro observation that E1,3 AT1a receptor mRNA is more efficiently translated than the E1,2,3 transcript (Example 12). Furthermore, this data demonstrates that, in RASMC, the majority of AT1a receptor protein is translated from a splice variant that makes up less than a quarter of the total AT1a receptor mRNA population. Given that E1,2,3 mRNA appears to be predominant in all tissues, it is probable that, in most instances, translation of the E1,3 mRNA contributes to the majority of AT1a receptor protein. As described in the Introduction, studies have demonstrated that alterations in splicing of the human AT1 receptor are associated with significant cardiovascular pathophysiology (Martin et al., 2001; Warnecke et al., 1999). These studies suggest that alternative splicing plays an important regulatory role in control of angiotensin receptor gene expression and contribute, directly or indirectly, to heart disease. However, while alternative splicing may play a critical role in regulation of the RAS, the mechanisms by which alternative splicing might regulate AT1 receptor expression remain unclear. There are two potential mechanisms by which splicing could regulate AT1a receptor expression. Firstly, AT1a receptor expression could be regulated by alterations in the relative proportion of E1,3 and E1,2,3 mRNA. Since the E1,3 mRNA is translated more efficiently than E1,2,3 mRNA, any increase in the proportion of the E1,3 variant in the AT1a receptor mRNA pool (even if total AT1a receptor mRNA levels do not change) could result in up-regulation of the receptor. Conversely, a decrease in the proportion of E1,3 mRNA could result in down-regulation. Applicants have previously shown that this first mechanism may account for tissue specific expression of AT1a receptors since Applicants found a tight correlation between E1,3 splice ratio [E1,3:(E1,3+E1,2,3)] and AT1 receptor number (Bmax) in Sprague Dawley rat tissues that predominantly express the AT1a receptor (Zhang et al., in press).

The second possibility is that the rate of translation of E1,3 and E1,2,3 mRNA may be differentially regulated. Translation of E1,2,3 mRNA has previously been shown to be regulated by cytosolic proteins which bind to the 5′UTR of the mRNA and inhibit its translation (Krishnamurthi et al., 1999; Wu et al., 2003b). In studies in the rat, a group of cytosolic proteins were shown to bind the E1,2,3 AT1a receptor 5′UTR by ultraviolet crosslinking assays. An inverse relationship was observed between changes in AT1 receptor density and the activity of these 5′UTR binding proteins in cytosolic extracts. This inverse relationship was observed after regulation of AT1 receptor by both dietary sodium (Krishnamurthi et al., 1998) and estrogen (Krishnamurthi et al., 1999; Wu et al., 2003a). In vitro translation assays of the AT1a receptor in wheat germ lysates showed that addition of these 5′UTR binding proteins reduced the rate of translation of the E1,2,3 AT1a receptor splice variant (Krishnamurthi et al., 1999). At present, the regulation of the E1,3 mRNA by these binding proteins has not been investigated, although preliminary data suggests that the affinity of these proteins for E1,3 5′UTR is significantly less than for the E1,2,3 5′UTR. Thus, it is possible that as a consequence of the differences in its' 5′ UTR, these cytosolic binding proteins are unable to regulate translation of the E1,3 AT1a receptor splice variant. Potentially, increases in the activity of the binding proteins could reduce the rate of translation of the E1,2,3 splice variant while leaving translation of the E1,3 splice variant unaffected.

It is important to note that these two potential mechanisms for regulation of AT1a receptor expression are not mutually exclusive. It is quite possible that both mechanisms occur simultaneously, and that there are complex interactions between the two processes. At present there is little direct evidence to support the involvement of either mechanism in regulation of AT1a receptor expression. However, it should be noted that expression of AT1a receptor mRNA is commonly measured using assays that are not splice variant-specific (e.g. Northern Blot, or RT-PCR with a single pair of primers). Thus, it is possible that in many instances an important contribution of splicing to AT1a receptor expression has been missed.

These data demonstrate that it is possible to reduce expression of the AT1a receptor using RNAi. While evidence from in vitro and cell culture systems suggests that any mRNA can be silenced using RNAi techniques (Elbashir et al., 2002), the ability to induce a reduction in protein expression may be limited by the rate of turnover of the specific mRNA and protein. Since the half-life of AT1 receptors, like most G protein coupled receptors, appears to be relatively long (14.8 h for the bovine AT1 receptor in adrenal cells (Ouali et al., 1997)), it was uncertain whether sufficient silencing of the AT1a receptor could be achieved by RNA interference. As shown in FIG. 24, treatment with S2E3 siRNA induced a marked reduction in both AT1a receptor mRNA levels and AT1a receptor protein, as measured by AT1 receptor binding. These data represent the first demonstration of the use of RNAi to reduce expression of a G protein-coupled receptor in mammalian cells.

The present data also demonstrate that, because of the highly sequence-specific nature of the interaction between the siRNA guide and its target sequence, it is possible to use RNAi to selectively reduce the expression of individual mRNA splice variants while leaving other splice variants intact in mammalian cells. RNAi of transcripts containing particular exons has previously been demonstrated in cultured Drosophila cells (Celotto and Graveley, 2002) and MDA231 breast cancer cells (Ge et al., 2003) respectively. The data described in this study extend these findings by demonstrating that, by targeting the junction between two exons, it is possible it is possible to selectively silence transcripts which do not contain a particular exon. Since alternative splicing makes a highly important contribution to the complexity of the proteome, and is regulated developmentally and in response to cellular stimuli (Rao and Howells, 1993; Xie and Black, 2001), investigation of the role of alternative splicing in many physiological processes is likely to become increasingly important. Use of splice variant-specific RNAi is likely to be an invaluable tool in these investigations.

In conclusion, Applicants have demonstrated that E1,3 AT1a receptor mRNA is more efficiently translated than E1,2,3 mRNA in RASMC. In these cells most AT1a recpetor protein was found to be transcribed from the E1,3 splice variant. Together, these data are the first demonstration of splice variant-specific RNAi in mammalian cells and the first use of RNAi technology to reduce expression of a G protein coupled receptor in mammalian cells.

### References

Caplen N J, et. al. Proc Natl Acad Sci USA 98:9742-7.
Cazzola M and Skoda R C (2000) Blood 95:3280-8.
Celotto A M and Graveley B R (2002) Rna 8:718-24.
Corvol P, et. al. Recent Prog Horm Res 50:287-308.
Elbashir S M, et. al. (2002) Methods 26:199-213.
Elton T S and Martin M M (2003) Trends Endocrinol Metab 14:66-71.
Ge B, et. al. (2003) J Biol Chem 278:2286-93.
Griendling K K, et. al. (1996) Annu Rev Pharmacol Toxicol 36:281-306.
Ji H, et. al. (1995) Proc Natl Acad Sci USA 92:9240-4.
Kakar S S, et. al. (1992a) Biochem Biophys Res Commun 185:688-92.
Kakar S S, et. al. (1992b) Biochem Biophys Res Commun 183:1090-6.
Kitami Y, et. al. (1992) Biochem Biophys Res Commun 188:446-52.
Klausner R D, et. al. (1993) Cell 72:19-28.
Krishnamurthi K, et. al. (1999) Endocrinology 140:5435-8.
Krishnamurthi K, et. al. (1998) Biochem Biophys Res Commun 245:865-70.
Langford K, et. al. (1992) Biochem Biophys Res Commun 183:1025-32.
Martin, M M, et. al. (2001) Mol Endocrinol 15:281-93.
Martin N P, et. al. (2003) J Biol Chem 278:45954-9.
Murphy T J, et. al. (1991) Nature 351:233-6.
Nickenig G and Harrison D G (2002) Circulation 105:530-6.
Ouali R, et. al. (1997) Endocrinology 138:725-33.
Rao S M and Howells R D (1993) J Biol Chem 268:22164-9.
Rouault T and Klausner R (1997) Curr Top Cell Regul 35:1-19.
Sandberg K and Ji H (2000) Semin Nephrol 20:402-16.
Sandberg K and Ji H (2001) Comp Biochem Physiol A Mol Integr Physiol 128:53-75.
Sandberg K (1992) J Biol Chem 267:9455-8.
Stroth U and Unger T (1999) J Cardiovasc Pharmacol 33 Suppl 1:S21-8; discussion S41-3.
Teerlink J R (1996) J Cardiovasc Pharmacol 27 Suppl 2:S 1-8.
Touyz R M and Schiffrin E L (2000) Pharmacol Rev 52:639-72.
Warnecke C, et. al. (1999) J Mol Med 77:718-27.
Wu Z, et. al. (2003a) Endocrinology 144:3251-61.
Wu Z, Zheng W and Sandberg K (2003b) Endocrinology 144:1350-6.
Xie J and Black D L (2001) Nature 410:936-9.
Zhang Y et. al. Example 12 (and in press, Gene).

### Example 14

### Translational Regulation of Angiotensin Type 1 a Receptor Expression and Signaling by Upstream AUGs in the 5′ Leader Sequence

The references cited in this example (by number) are those specified at the end of this example.

In this example, Applicants show that expression of the rat angiotensin type I a receptor (AT1aR) is regulated by 4 upstream AUGs present in the 5′ leader sequence (5′LS). Disruption of all 4 upstream AUGs (QM) results in 2-3-fold higher levels of AT1R densities in transiently transfected rat aortic smooth muscle cells (A10) and stably transfected Chinese Hamster Ovary (CHO) cells. Cells expressing QM have 5-fold higher levels of Ang II-induced inositol phosphate production than wild type (WT). Polysome analysis showed that QM mRNA is present in heavier fractions than the WT transcript and 5.7-fold more AT1R protein is produced by in vitro translation (IVT) from QM transcripts compared to WT. The AT1aR is comprised of 3 exons. Exon 3 (E3) encodes the entire open reading frame and 3′ untranslated region. Exons 1 and 2 (E1 and E2) and 52 nucleotides of E3 encode the 5′LS. The AUGs in both exons contribute to the inhibitory effect on AT1R expression but not to the same degree. Disruption of the AUGs in exon 2 (DM2) relieves half of the inhibition while disruption of the AUGs in exon 1 (DM1) is without effect. DM2 results in levels of receptor expression and translation that are indistinguishable from the alternative splice variant, E1,3, which Applicants previously showed was more efficiently translated than the E1,2,3 transcript. Individual mutations revealed that only the 4th AUG increased AT1R translation. In conclusion, all 4 AUGs present in the 5′LS function cumulatively to suppress AT1aR expression and signaling by inhibiting translation. These data also show that both AUGs in E2 contribute to the inhibitory cis element present in this alternatively spliced exon.

In the majority of eukaryotic mRNAs, the first AUG downstream from the 5′ cap site is the start of translation (1). However, in mRNAs that code for key regulatory proteins such as transcription factors, protooncogenes, and key signaling molecules, AUGs are commonly found upstream of the start of translation. Upstream AUGs can play a critical role in control of gene expression by causing ribosomal pausing or by forming a translation-competent ribosome that can initiate, terminate and re-initiate. Both these mechanisms can lead to reduced translation of the downstream open reading frame. Alternatively, an N-terminally extended protein can be synthesized from initiation at the upstream AUG thereby competing with translation at the down stream open reading frame (2).

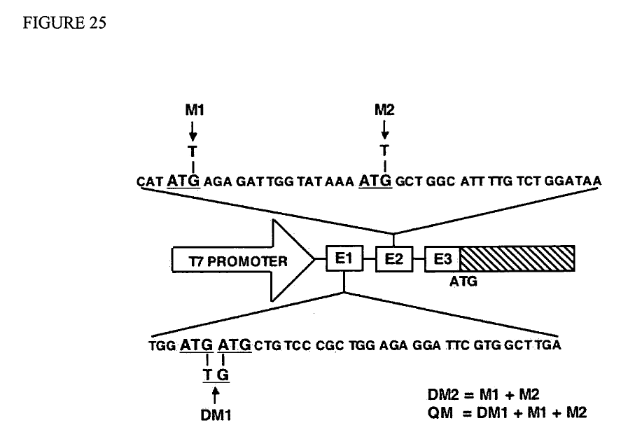

Applicants have been studying the post-transcriptional regulation of the type 1 angiotensin receptor (AT1R) (3), which is a G protein coupled receptor that plays a critical role in regulating blood pressure and fluid homeostasis and antagonists of this receptor are widely used to control hypertension and reduce the rate of progression of cardiovascular and renal disease (4). The rat AT1aR is comprised of 3 exons (FIG. 25). E3 harbors the entire open reading frame and the 3′ untranslated region while the 5′LS is comprised of exons 1, 2 (E1 and E2) and 52 nucleotides of E3. There are 4 upstream AUGs present in the 5′LS, 2 in E1 and 2 in E2. E2 is alternatively spliced in a tissue specific manner and contains an unidentified cis element that is inhibitory to receptor expression (5). In this study, Applicants investigated the function of the upstream AUGs present in the 5′LS on AT1R expression and signal transduction in transiently transfected A10 rat aortic smooth muscle cells and in stably transfected Chinese Hamster Ovary (CHO) cells. Applicants also studied the role of upstream AUGs on receptor translation by polysome analysis in transfected cells and by in vitro translation assays.

Experimental Procedures

Site-directed mutagenesis: The AUGs in the 5′LS of the AT1aR cloned into the pcDNA5/FRT vector (Invitrogen) were subjected to site-directed mutagenesis using the Quick Change site-directed mutagenesis system (Stratagene).

A10 cell culture and transient transfections: A10 cells were cultured in Dulbecco's Modified Essential Medium supplemented with 4 mM L-glutamine, 10% fetal bovine serum (FBS) and antibiotics (100 IU/ml penicillin, 100 μg/ml streptomycin). When cells were 60-75% confluent, 20 μg of plasmid DNA per 100 mm dish was transiently transfected by the calcium phosphate method (Invitrogen).

CHO cell culture and stable transfections: CHO cells were cultured in Ham's F-12K with 1.5 g/L sodium bicarbonate, 2 mM L-glutamine, 10% FBS and antibiotics (as above). When cells were 60-75% confluent, 20 μg of plasmid DNA per 100 mm dish was transfected by the calcium phosphate method (Invitrogen). Individual clones were cultivated as described previously (6).

AT1R radioligand binding: A10 and CHO cell membranes were used in radioligand binding assays using 125I-[Sar1,Ile8]Ang II and a Brandel cell harvester as described (7). Kd and Bmax values from Scatchard plots were determined using the nonlinear regression analysis program, PRISM.

RNase protection assay: Total RNA was isolated and E1,3 and E1,2,3 mRNA were measured by RNA protection assay as previously described (3) using a probe based on the coding region and thus common to both transcripts (FIG. 25). In brief, the cDNA encoding the rat AT1aR coding region in the pBluescript II vector (Stratagene) was linearized with EcoR1 and transcribed in vitro with T7 RNA polymerase to yield a 380 bp protected cRNA fragment after hybridization with 3 μg of total RNA followed by RNase digestion according to the RPA III protocol (Ambion). The probe for β-actin was generated from pTRI-Actin-Mouse with T7 RNA polymerase and yielded a 245 bp cRNA fragment. Radioactive signals were detected by a phosphoimager after electrophoresis on a 5% acrylamide gel.

Real-time PCR: Total RNA was extracted using TRIzol reagent (Life Technologies). First strand cDNA was made from total RNA using iScript cDNA synthesis kit (BioRad) with MMLV RNase H+reverse transcriptase, oligo(dT) and random hexamers. Quantitations of specific mRNAs and 18S rRNA (for control) were performed by real-time PCR using the ABI Prism 7700 Sequence Detection System (Perkin Elmer Applied Biosystems). The PCR reaction mixture consisted of RNase free water, TaqMan Universal PCR Master Mix (Perkin Elmer Applied Biosystems) and 300 nM specific primers and 10 μM probe (Forward primers: 119F (E1,2,3), 5′-CCA CAT TCC CTG AGT TAA CAT ATG A-3′ (SEQ ID NO. 78) and 114F (E1,3), 5′-CTC TGC CAC ATT CCC TGG TC-3′(SEQ ID NO. 79); Reverse primer: 310R (E1,2,3 & E1,3), 5′-TCT TTT GAT ACC ATC TTC AGC AGA A-3′) (SEQ ID NO. 80); and Probe: 232T (E1,2,3 & E1,3), 6 FAM-TCG AAT AGT GTC TGA GAC CAA CTC AAC CCA-TAMRA) (SEQ ID NO. 81), and cDNA samples. PCR conditions were optimized for the probe (232T) and both sets of primers (119F & 310R and 114F & 310R) using control cDNAs. The specificity of these primers were confirmed in CHO cells stably expressing E1,3 or E1,2,3. That is, Applicants did not detect any amplified products using E1,3 specific primers in the E1,2,3 expressing cells and vice versa. The expression of 18S rRNA, E1,3 and E1,2,3 mRNA in each sample was quantitated using respective primers. PCR reactions without reverse transcription were included to control for contamination by genomic DNA. The standard curves for 18S rRNA, E1,3 and E1,2,3 mRNA were made from a series of ten times dilutions (53, 54, 55, 56, 57, and 58) for each cDNA. The cell and tissue levels of these cDNAs were calculated based on the standard curves.

Inositol phosphate (IP) assay: CHO cells stably expressing WT and QM were cultured to 70% confluence in 24-well plates before treated for 16 hours with DMEM media containing 3 μCi/ml myo-[3H]-inositol (Amersham). Ang II-induced IP production was assayed as described (8).

In vitro translation (IVT): WT and mutated cDNAs were subcloned into the pcDNA5/FRT expression vector (Invitrogen). Plasmid DNAs were linearized by Xho I digestion and in vitro transcribed into capped RNA (T7 mMessage mMachine, Ambion). RNA (1 μg) was translated in wheat germ extracts (Promega) in the presence of 35S-methionine (Amersham), as described (9).

Polynomal distribution analysis. Polysome analysis was performed as described (3) and is based on the principle that the largest polysomes are denser and therefore, will sediment faster through a sucrose gradient than monosomes or free ribosomal subunits not bound to mRNA (10). The amount of WT or QM mRNA in each fraction was determined by RNase protection assay as described (3). The same probe was used in the RPA assay for WT and QM because it bound to an identical region in both transcripts. Sample variation in cytoplasmic levels of AT1R mRNA was controlled by normalizing the AT1R mRNA recovered in each fraction to the total amount of AT1R mRNA recovered from the entire fractionation.

RNase Protection

Statistics: Data are expressed as means±SEM. Statistical significance of the differences between groups was determined by student's t test.

Results

Effect of disrupting all four upstream AUGs on AT1R densities

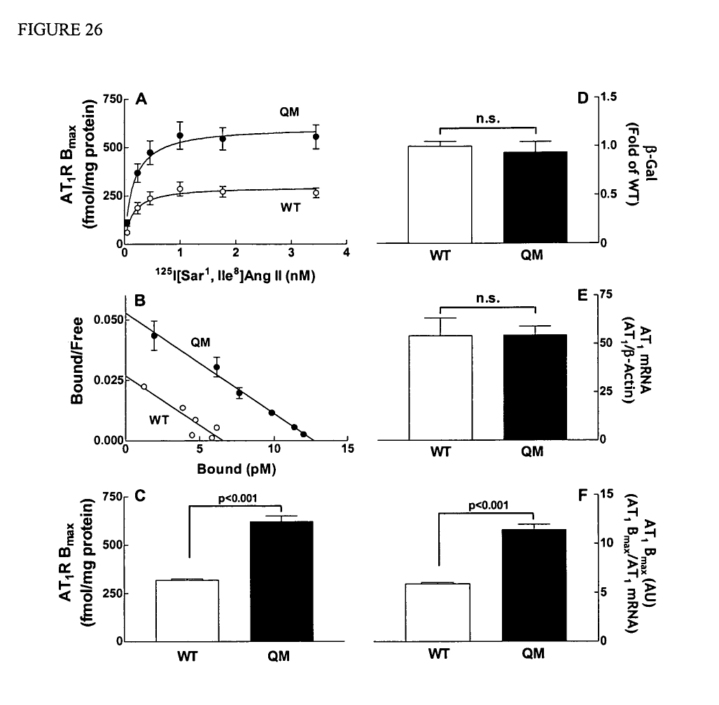

There are 4 upstream AUGs in the 5′LS of the E1,2,3 AT1aR mRNA transcript (WT) (FIG. 25). All four were disrupted by site-directed mutagenesis to create QM (FIG. 25). To determine the functional differences between WT and QM, A10 cells were transiently transfected with WT or QM plasmid DNA before radioligand saturation isotherms was performed (FIG. 26A). Scatchard plots (FIG. 26B) of saturation isotherms revealed that AT1R densities (Bmax) were 2.0-fold higher in A10 cells transfected with QM compared to WT [Bmax (fmol/mg): WT, 319±7.8 vs QM, 621±30, n=3, p<0.001] (FIG. 26C). The ligand binding affinities were indistinguishable between WT and QM [Kd (nM): WT, 0.14±0.02 vs QM, 0.17±0.04]. In B-GAL co-transfection experiments, no differences in B-GAL transfection efficiencies were observed [Arbitrary units: WT, 1.0±0.05; QM, 0.93±0.11] (FIG. 26D). When AT1aR mRNA levels were determined by RNase protection assay, no differences in AT1aR mRNA levels were found between WT and QM (FIG. 26E). Accordingly, when WT and QM Bmax values were normalized to mRNA levels, QM still expressed 2-fold higher AT1R densities than WT (FIG. 26F).

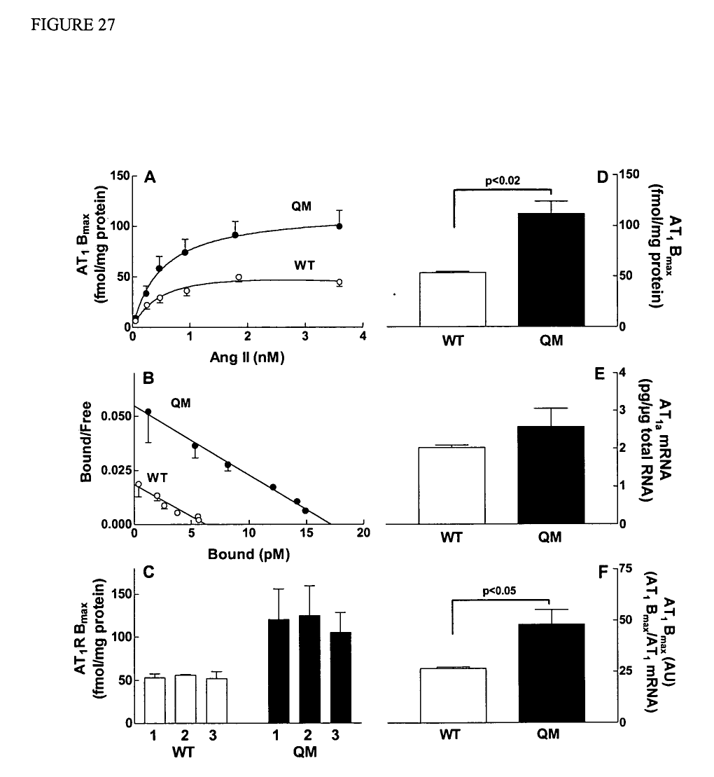

To further characterize the QM mutant, QM and WT plasmids were stably transfected into CHO cells. Three independent and randomly selected clones were isolated for each plasmid. Saturation isotherms (FIG. 27A) and Scatchard plots (FIG. 27B) were performed on each clone. All three individual QM clones expressed higher AT1R densities than all three WT clones (FIG. 27C). When the Bmax vales from all three WT and QM clones were averaged (FIG. 27D), QM exhibited 2.1-fold higher AT1R densities compared to WT [Bmax (fmol/mg): WT, 54±1.2 vs QM, 112±12, n=3, p<0.02] (FIG. 27D). No differences in AT1R mRNA levels were detected by real-time PCR (FIG. 27E). When WT and QM Bmax values were normalized to AT1R mRNA levels, QM expressed 1.8-fold higher AT1R densities than WT expressing cells (FIG. 27F).

Effect of Disrupting All 4 Upstream AUGs on IP Signaling

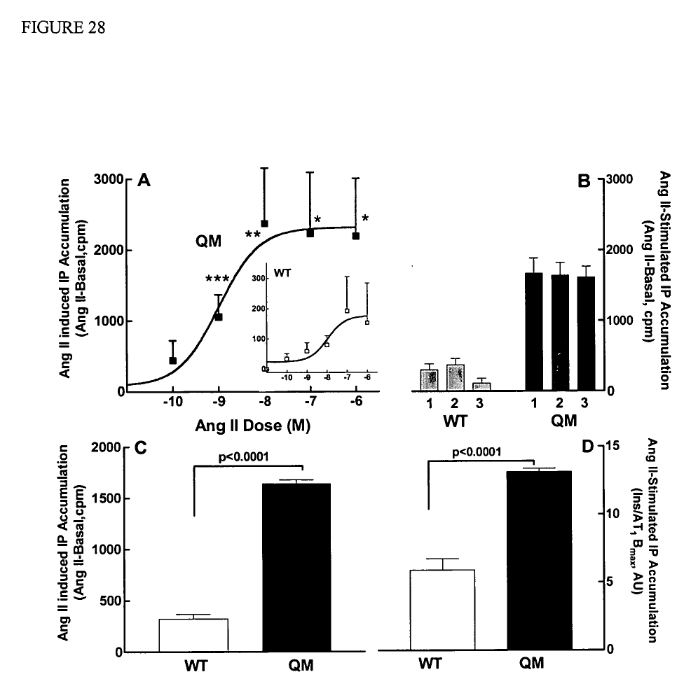

To determine if the differences in receptor densities affected Ang II signal transduction pathways, Ang II-induced IP production was determined in the CHO cells stably expressing WT and QM. An Ang II dose response curve on CHO cells expressing WT (FIG. 28A, inset) and QM (FIG. 28A) showed that 100 nM Ang II resulted in maximum accumulation of IP after 20 minutes. All three QM clones produced significantly more IP in response to 100 nM Ang II stimulation than all three WT clones (FIG. 28B); QM expressing cells produced 5-fold higher levels of IP compared to the WT clones [IP (cpm): WT, 322±47 vs QM, 1642±37, p<0.0001](FIG. 28C). When normalized to AT1R Bmax, QM expressing cells produced 2.2-fold higher levels of IP than WT expressing cells (FIG. 28D).

Effect of Disrupting all 4 Upstream AUGs on Translation

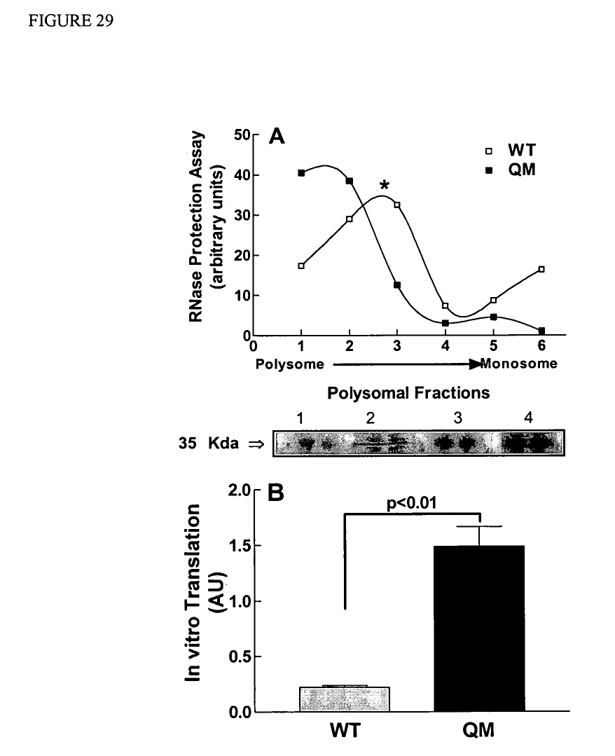

Polysome distribution profiles of AT1R mRNA were performed on WT and QM expressing CHO cells to compare WT and QM translational efficiencies in cells. The majority of the AT1R mRNA was located in fractions 2 and 3 in the WT expressing cells while the AT1R mRNA was shifted to the denser and more actively translated polysome fractions (fractions 1 and 2) in the QM expressing cells (FIG. 29A). To determine if QM was also translated in vitro more rapidly than WT, capped WT and QM RNAs were IVT in wheat germ extracts (FIG. 29B, inset). These IVT assays showed that 5.7-fold more AT1R protein was synthesized by QM compared to WT (FIG. 29B).

Relative Contribution of Upstream AUGs in E1 and E2 on Inhibition of AT1R Densities and IVT

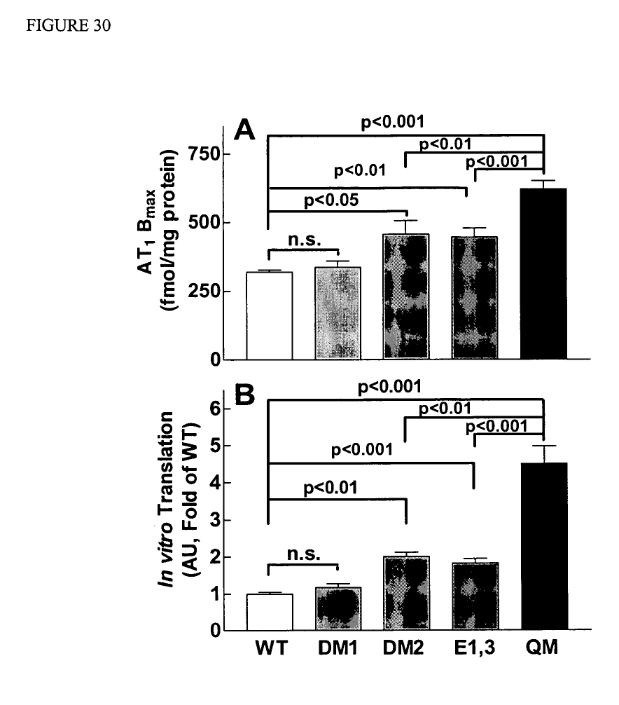

Applicants used site-directed mutagenesis to create DM1, in which the 2 AUGs in E1 were disrupted and DM2, in which the 2 AUGs in E2 were mutated (FIG. 25). Radioligand binding analysis showed that AT1R densities increased by 1.4-fold in DM2 expressing cells compared to WT, whereas no differences in AT1R densities were observed between DM1 and WT transfected cells (FIG. 30A). The levels of AT1R expression in DM2 expressing cells were not as high as QM; QM resulted in 1.4-fold higher receptor levels than DM2 and 2.0-fold higher levels than WT.

To determine the contribution of AUGs in El and E2 to translational inhibition, IVT assays were performed. No differences in AT1R translational efficiency were observed between the DM1 and WT transcripts whereas DM2 resulted in a 2.0-fold increase in AT1R protein levels (FIG. 30B). The level of IVT in DM2 expressing cells was not as high as QM; QM resulted in 2.3-fold higher levels than DM2 and 4.5-fold higher levels than WT.

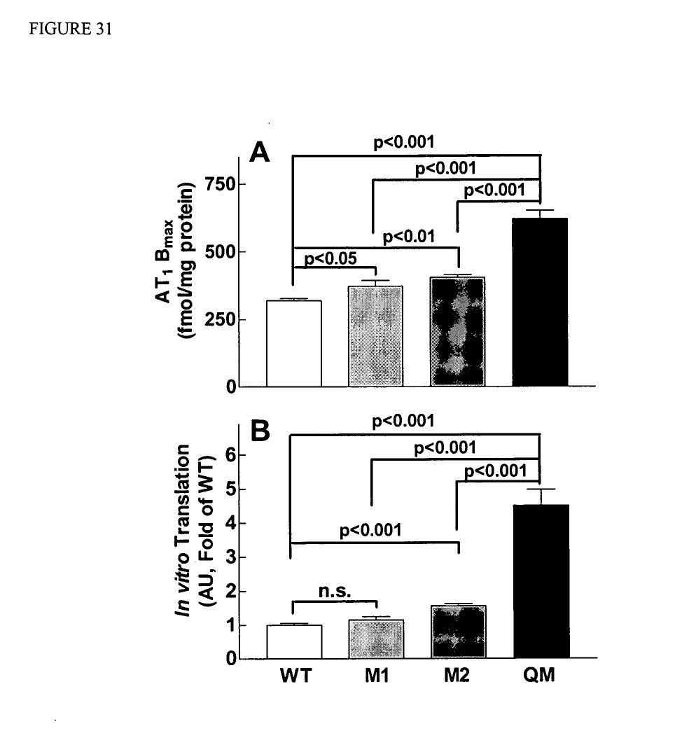

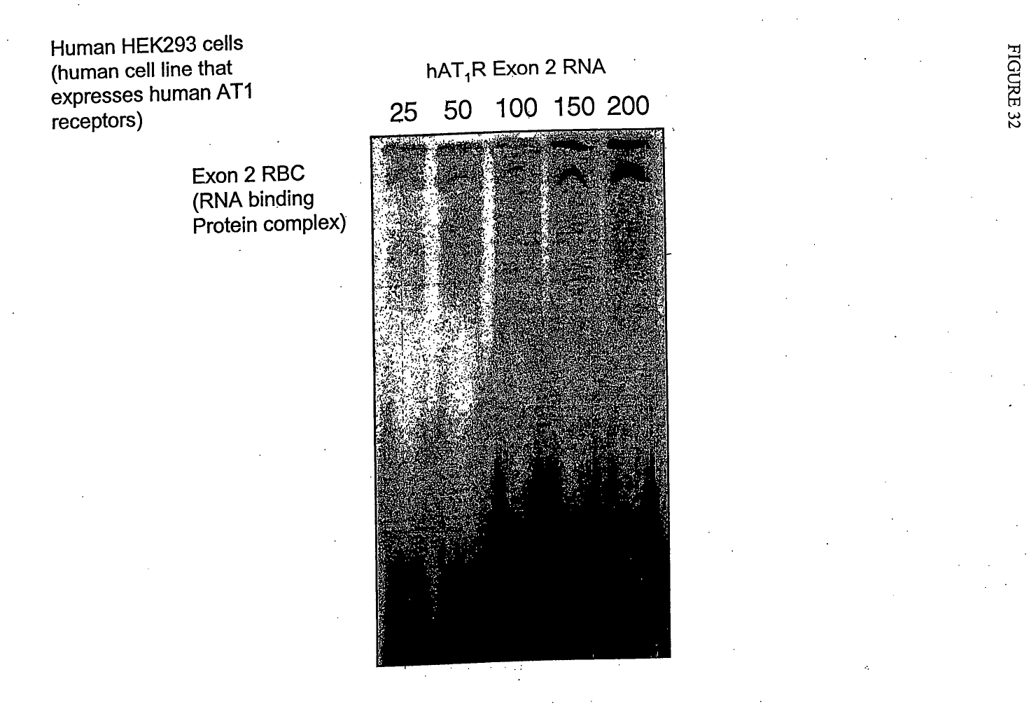

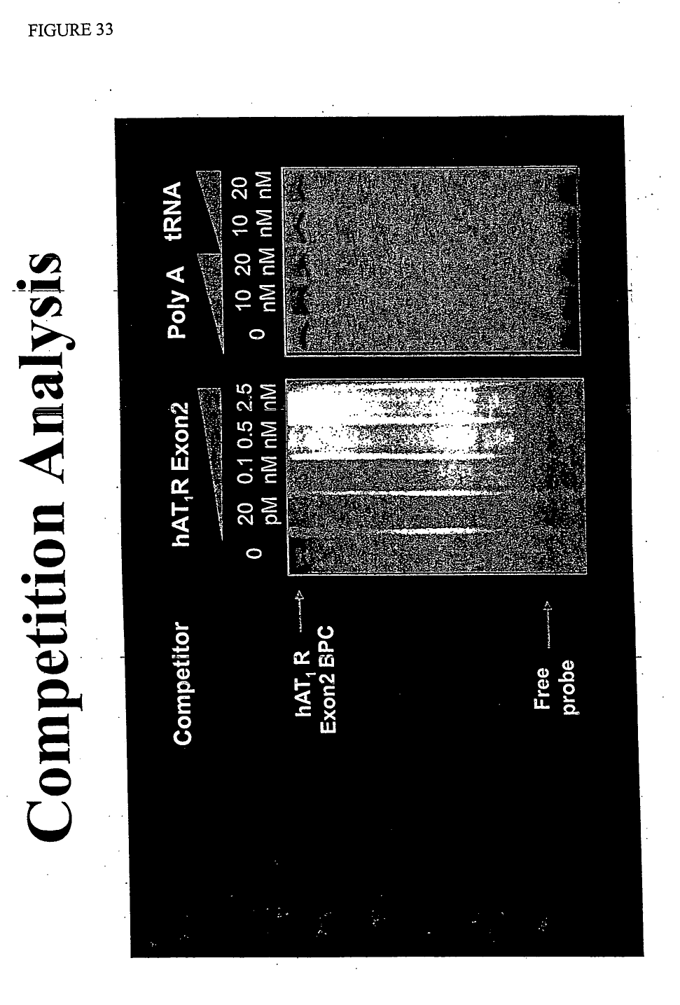

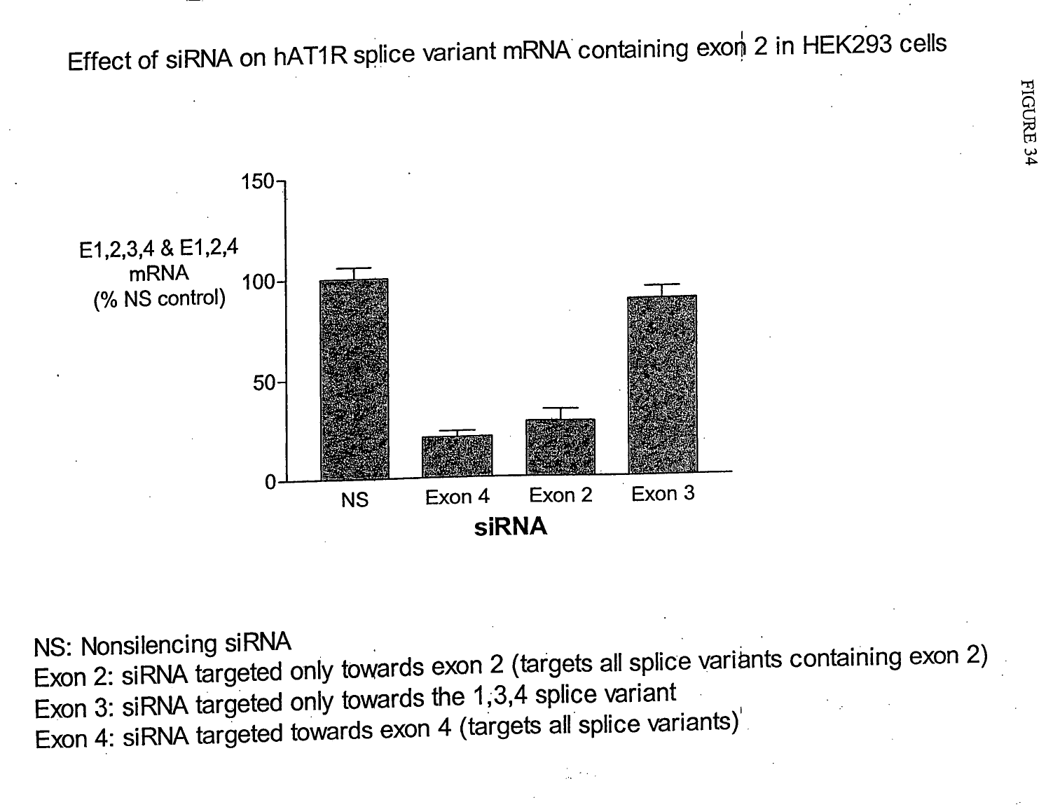

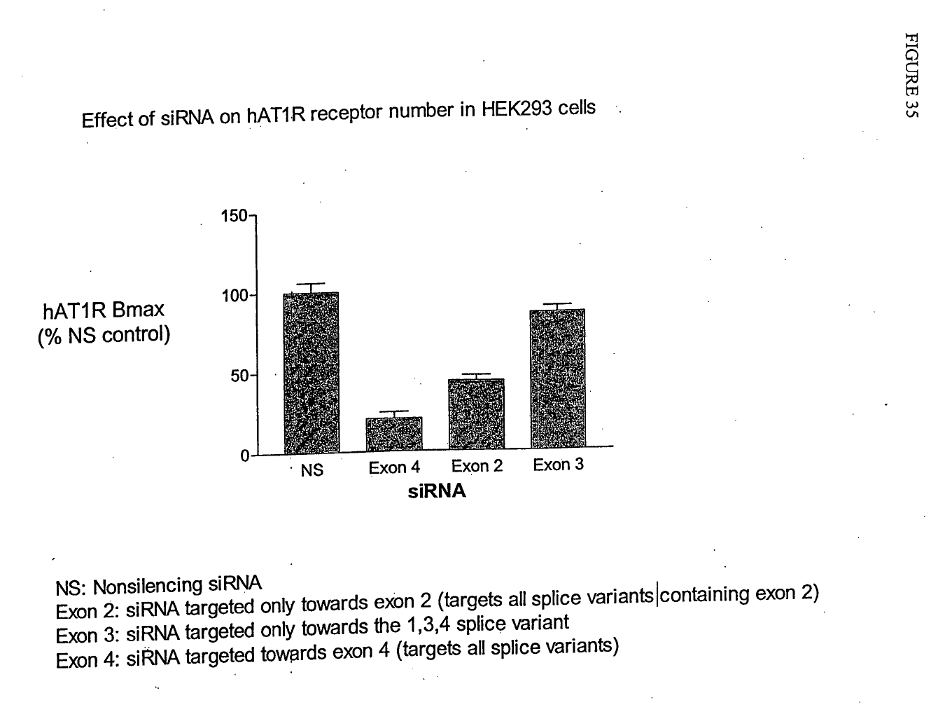

To further dissect the contribution of individual upstream AUGs in inhibiting AT1R expression, the two AUGs in E2 were individually mutated by site-directed mutagenesis to create M1 and M2 (FIG. 25). Radioligand binding studies showed an incremental increase in AT1R densities for M1 (1.1-fold) and a 1.3-fold increase for M2 (FIG. 31A). IVT assays showed a similar trend; M2 resulted in a 1.6-fold increase in IVT whereas IVT of M1 was indistinguishable from WT (FIG. 31B). Since relief from translational repression was only observed in M2 and only this 4th AUG was in optimal Kozak consensus sequence (the +4 position is G and the −3 position is A (1)), Applicants investigated if initiation could occur at this 4th AUG by disrupting the in frame stop codon (TAA-TAT) (SEQ ID NO. 82), which is also in frame with the down stream open reading frame encoding the AT1R. A larger protein encompassing the extra 44 amino acids, however, was not detectable by IVT (data not shown).

Comparison of the Effects of DM2 and the Splice Variant, E1,3, on AT1R Expression and IVT

The two AT1aR splice variants (E1,2,3 and E1,3) differ only in the length of their 5′LS. Thus, both transcripts code for identical proteins. Applicants recently showed that E2 contains an inhibitory RNA cis element, which results in reduced AT1R expression and signaling (5). As found previously, radioligand binding assays showed that AT1R densities in E1,3 expressing cells were 1.4-fold higher than WT expressing cells, which was indistinguishable from DM2 (FIG. 30A). IVT assays showed a similar result; the E1,3 transcript was translated 2.0-fold more efficiently than WT and no differences were observed between the levels of IVT for DM2 and E1,3 (FIG. 30B).

Discussion

Applicants show that disruption of all four AUGs leads to marked increases in AT1R binding in both transiently transfected and stably transfected cells. The finding that β-gal was equivalently expressed in WT and QM co-transfection experiments and that at least 3 independent and randomly selected stable clones of QM expressed higher levels of AT1R binding when compared to 3 independent and randomly selected WT clones rules out the likelihood that differences in transfection efficiencies or different sites of integration account for these results. Scatchard analysis of radioligand binding studies indicates that the increase in AT1R binding in cells expressing QM compared to WT arises from an increase in receptor density rather than increased receptor affinity, which is consistent with the fact that WT and QM code for identical proteins. The increase in AT1R densities in QM expressing cells in unlikely due to increased QM mRNA levels since AT1R densities were still significantly higher after normalization to AT1R mRNA levels in both transiently and stably expressing cells.

The functional significance of the 2-fold increase in AT I aR densities in QM expressing cells is illustrated by the observation that Ang II-stimulated IP production is markedly higher in QM compared to WT expressing cells. This data also support studies showing a close correlation between AT1R density and signal transduction in vascular smooth muscle cells (11). The finding that in response to Ang II stimulation, QM expressing cells produce more IP than WT expressing cells when normalized to AT1R Bmax is consistent with the expected amplification of signaling that occurs in signal transduction cascades.

The observation that QM mRNA was associated with heavier polysome fractions (and thus with more actively translated mRNAs) than WT mRNA suggests that upstream AUGs are inhibitory to translational efficiency in cells. This conclusion is further supported by IVT assays in which QM is translated in vitro with greater efficiency than WT.

Mutagenesis studies show that disruption of both AUGs in El offers no liberation from the 5′LS inhibition of AT1R expression. However, the fact that the DM2 mutant results in AT1R expression levels that are half the levels of QM indicates that the AUGs in El contribute. One possibility is that part of the effect of QM involves a conformation change in the 5′LS that only occurs when all four AUGs are disrupted. Thus, even though disruption of the AUGs in El had no effect on AT1R binding or translation, their disruption contributed to QM efficient translation.

Disruption of the 2 AUGs in E2 completely relieves the effects of the E2 inhibitory cis element on receptor expression and signaling (5). These findings suggest that the 3rd and 4th AUGs comprise a major component of the inhibitory cis element within E2 and thus, may be the RNA cis elements that contribute to control of AT1R regulation by alternative splicing. By controlling the degree of alternative splicing of E1,2,3, an additional level of control is available by which the cell can tightly regulate the expression and function of the AT1R. Applicants recently showed that splicing is regulated in a tissue-specific manner and that the splice ratio of E1,3 to E1,2,3 tightly correlates with tissue specific differences in AT1R expression (12). Thus, alternative splicing of the AT1R is one more mechanism by which AT1R expression can be controlled in addition to regulation by other mechanisms including transcription, mRNA stability, RNA binding proteins, receptor desensitization, ligand-mediated receptor internalization and receptor recycling (3,13-15).

Although DM2 and E1,3 result in the same levels of AT1R expression, this finding does not rule out the presence of additional inhibitory cis elements within E2; it is possible that inhibitory cis elements exist which are distinct from the two AUGs but are disrupted by forming DM2. In this regard, Applicants have recently reported that deletion of the loop in a putative hairpin in E2 markedly relieved the translational repression of E2 (12).

The finding that a longer AT1R was not detected by IVT when the in frame stop codon in E2 was disrupted suggests the 4th upstream AUG inhibits AT1aR expression by ribosomal pausing rather than by initiation, termination and re-initiation. In this regard, the rat AT1aR is distinct from the human AT1R, in which initiation can occur at an upstream AUG in the E1,3,4 splice variant resulting in a longer form of the receptor that is functionally distinct from the short form (16). The finding that M2 caused a significant increase in IVT while M1 or the 2 AUGs in E1 (DM1) did not, suggests ribosomal pausing is greatest at this 4th AUG and is consistent with the observation that this 4th AUG (FIG. 25) is the only one in optimal Kozak consensus sequence.

In summary, these data indicate that upstream AUGs in both E1 and E2 in the 5′LS of the AT1aR act cumulatively to repress receptor expression and signaling by inhibiting translation. The upstream AUGs in E1 require the presence of the upstream AUGs in E2 to be inhibitory whereas the upstream AUGs in E2 are inhibitory in and of themselves. However, the inhibitory effects of AUGs in E2 are amplified by the presence of the upstream AUGs in E1, suggesting 5′LS secondary structure is also important to the translational repression. Translational repression by these upstream AUGs is most likely due to ribosomal pausing rather than initiation since a longer form of the AT1R was not detected when the in frame stop codon in E2 was disrupted. In addition, these data suggest that the 3rd and 4th upstream AUGs are part of the inhibitory cis element present in E2 and therefore, may be responsible for regulating AT1aR expression by alternative splicing.

### References Cited in Example 14

1. Kozak, M. (1999) Gene 234, 187-208
2. Meijer, H. A., and Thomas, A. A. (2002) Biochem J 367, 1-11
3. Wu, Z., et. al. (2003) Endocrinology 144, 3251-3261
4. Sandberg, K., and Ji, H. (2000) Semin Nephrol 20, 402-416.
5. Zhang, Y., et. al. (2003) FASEB Annual Meeting of Experimental Biology, Abstract #7802
6. Tian, Y., et. al. (1996) Am J Physiol 270, E831-839
7. Ji, H., et. al. (1994) J Biol. Chem. 269, 16533-16536
8. Vanderheyden, P. M., Fierens, et. al. (1999) British Journal of Pharmacology 126, 1057-1065
9. Ji, H., et. al. (2000) Meth Mol Med 51, 171-192
10. Davies, E., and Abe, S. (1995) Methods Cell Biol. 50, 209-222
11. Lassegue, B., et. al. (1995) Mol. Pharmacol. 48, 601-609
12. Zhang, Y., et. al. (2004) (Example 12 and Gene in press)
13. Thekkumkara, T. J., et. al. (1998) Biochem. J 329, 255-264
14. Xu, K., and Murphy, T. J. (2000) J Biol Chem 275, 7604-7611
15. Thomas, W. G. (1999) Regul Pept 79, 9-23
16. Martin, M. M., et. al. (2001) Molecular & Cellular Endocrinology 183, 81-91

### Example 15

### Translational Control of Human Angiotensin Type 1 Receptor (AT1R) Splice Variants in HEK 293 Cells

Representative human siRNA designs that would target various human AT1 receptor splice variants are listed below.

Targeting all splice variants (E1,2,3,4; E1,3,4; E1,2,4; E1,4)

3′-GAAGCCUGCACCAUGUUUUdTdT-5′
5′-dTdTCUUCGGACGUGGUACAAAA-3′
3′-CCUGUACGCUAGUGUGUUUdTdT-5′
5′-dTdTGCACAUGCGAUCACACAAA-3′

Targeting exon 3-containing splice variants (E1,2,3,4; E1,3,4)

3′-GUCAACUGACAGUCCAAAGdTdT-5′
5′-dTdTCAGUUGACUGUCAGGUUUC-3′
3′-CUGACAGUCCAAAGGCUCCdTdT-5′
5′-dTdTGACUGUCAGGUUUCCGAGG-3′

Targeting exon 2-containing transcripts (E1,2,3,4; E1,2,4)

3′-UGGCUGGGUUUUUAUCUGAdTdT-5′
5′-dTdTACCGACCCAAAAAUAGACU-3′
3′-CUCACUGAUGCCAUCCCAGdTdT-5′
5′-dTdTGAGUGACUACGGUAGGGUC-3′
3′-GCUGGGUUUUUAUCUGAAUUU-5′
5′-ACCGACCCAAAAAUAGACUUA-3′

Targeting the E1,3,4 splice variant only

3′-GGCGCGGAUGAAGAAAAUGUU-5′
5′-CCCCGCGCCUACUUCUUUUAC-3′

Targeting the E1,2,4 splice variant only

3′-GCACCAGGUGUAUUUGAUAUU-5′
5′-GCCGUGGUCCACAUAAACUAU-3′
3′-UCGGCACCAGGUGUAUUUGUU-5′
5′-UCAGCCGUGGUCCACAUAAAC-3′

Targeting the E1,4 splice variant only

3′-GCGCGGGUGUAUUUGAUAUUU-5′
5′-CCCGCGCCCACAUAAACUAUA-3′
3′-GGGCGCGGGUGUAUUUGAUUU-5′
5′-GCCCCGCGCCCACAUAAACUA-3′

Targeting the E1,2,3,4 splice variant only

3′-GUCGGCACCAGAUGAAGAAdTdT-5′
5′-dTdTCAGCCGUGGUCUACUUCUU-3′
3′-GCACCAGAUGAAGAAAAUGUU-5′
5′-GCCGUGGUCUACUUCUUUUAC-3′

The publications and other materials used herein to illuminate the background of the invention, and in particular, cases to provide additional details respecting the practice, are incorporated by reference in full.

Equivalents

Those skilled in the art will recognize, or be able to ascertain using no more than routine experimentation, many equivalents to the specific embodiments of the invention described herein. Such equivalents are intended to be encompassed by the following claims.

1. An expression vector comprising a nucleic acid encoding an siRNA or a hairpin RNA that specifically targets a region of an AT1R gene.

2. The expression vector of claim 1, wherein the AT1R gene is a human AT1R gene.

3. The expression vector of claim 1, wherein the AT1R gene is a rat AT1R gene.

4. The expression vector of claim 1, wherein the region is either an exon of the AT1R gene or a juncture between two different exons of the AT1R gene.

5. The expression vector of claim 1, wherein the siRNA or the hairpin RNA specifically targets a splice variant of the AT1R gene.

6. An isolated siRNA or a hairpin RNA that specifically targets a splice variant of an AT1R gene.

7. An expression vector comprising a nucleic acid encoding an antisense sequence that attenuates the expression of a splicing variant of an AT1R gene.

8. An isolated antisense nucleotide sequence that specifically targets a splice variant of an AT1R gene.

9. A method of modulating angiotensin II-mediated signaling in a cell, comprising contacting a cell with an agent that modulates translational efficiencies of one or more splicing variant of an AT1R gene.

10. The method of claim 9, wherein the AT1R gene is a human AT1R gene.

11. The method of claim 9, wherein the splicing variant is a naturally occurring splicing variant of the AT1R gene.

12. The method of claim 9, wherein the splicing variant is a recombinantly generated splicing variant of the AT1R gene and comprises one or more mutation.

13. The method of claim 9, wherein the cell is a mammalian cell.

14. The method of claim 9, wherein the cell is a human cell.

15. A method of screening for an agent that reduces angiotensin II-mediated signaling in a cell, comprising identifying an agent that decreases translational efficiency of one or more splicing variant of an AT1R gene.

16. The method of claim 15, wherein the AT1R gene is a human AT1R gene.

17. The method of claim 15, wherein said agents are siRNAs.

18. The method of claim 15, wherein said agents are small molecules.

19. The method of claim 15, wherein said agents modulate the activities of RNA binding proteins that interact with 5′ leader sequence of an AT1R mRNA.

20. A method of treating or preventing an angiotensin II-mediated disorder in a subject, comprising administering to the subject a therapeutically effective amount of an agent that modulates translational efficiency of one or more splicing variant of an AT1R gene.

21. The method of claim 20, wherein the disorder is hypertension.

22. The method of claim 20, wherein the subject is known to have or suspected of having hypertension.

23. A pharmaceutical preparation comprising an agent that modulates the alternative splicing resulting in AT1R transcripts, and promotes the alternative splicing resulting in a specific AT1R transcript.


SEQUENCE LISTING (117 sequences)

SEQ ID NO: 1 - RNA, 21 nt, Artificial Sequence
  sense strand of siRNA example
cuggucaaguggauuucgauu

SEQ ID NO: 2 - RNA, 21 nt, Artificial Sequence
  antisense strand of siRNA example
ucgaaauccacuugaccaggg

SEQ ID NO: 3 - DNA, 25 bp, Artificial Sequence
  forward primer 119F
ccacattccctgagttaacatatga

SEQ ID NO: 4 - DNA, 20 bp, Artificial Sequence
  forward primer 114F
ctctgccacattccctggtc

SEQ ID NO: 5 - DNA, 25 bp, Artificial Sequence
  reverse primer 310R
tcttttgataccatcttcagcagaa

SEQ ID NO: 6 - DNA, 30 bp, Artificial Sequence
  Probe 232T
tcgaatagtgtctgagaccaactcaaccca

SEQ ID NO: 7 - DNA, 25 bp, Artificial Sequence
  Forward primer 119F
ccacattccctgagttaacatatga

SEQ ID NO: 8 - DNA, 20 bp, Artificial Sequence
  Forward primer 114F
ctctgccacattccctggtc

SEQ ID NO: 9 - DNA, 25 bp, Artificial Sequence
  reverse primer 310R
tcttttgataccatcttcagcagaa

SEQ ID NO: 10 - DNA, 30 bp, Artificial Sequence; identical to SEQ ID NO: 6
  probe 232T

SEQ ID NO: 11 - DNA, 25 bp, Artificial Sequence
  forward primer 119F
ccacattccctgagttaacatatga

SEQ ID NO: 12 - DNA, 20 bp, Artificial Sequence
  forward primer 114F
ctctgccacattccctggtc

SEQ ID NO: 13 - DNA, 25 bp, Artificial Sequence
  reverse primer 310R
tcttttgataccatcttcagcagaa

SEQ ID NO: 14 - DNA, 30 bp, Artificial Sequence; identical to SEQ ID NO: 6
  probe 232T

SEQ ID NO: 15 - DNA, 21 bp, Artificial Sequence
  Target No. 1 5′ to 3′
tctgccacattccctggtcaa

SEQ ID NO: 16 - DNA, 21 bp, Artificial Sequence
  Sense strand of siRNA to Target No. 1
tgccacauugggaggugaauu

SEQ ID NO: 17 - RNA, 21 nt, Artificial Sequence
  Anti-sense strand of siRNA to Target No. 1
uugaccugggaauguggcaga

SEQ ID NO: 18 - DNA, 21 bp, Artificial Sequence
  Target No. 2 5′ to 3′
ctgccacattccctggtcaag

SEQ ID NO: 19 - RNA, 21 nt, Artificial Sequence
  Sense strand of siRNA to Target No. 2
gccacauucccuggucaaguu

SEQ ID NO: 20 - RNA, 21 nt, Artificial Sequence
  Anti-sense strand of siRNA to Target No. 2
cuugaccugggaauguggcag

SEQ ID NO: 21 - DNA, 21 bp, Artificial Sequence
  Target No. 3  5′ to 3′
tgccacattccctggtcaagt

SEQ ID NO: 22 - RNA, 21 nt, Artificial Sequence
  Sense strand of siRNA to Target No. 3
ccacauucccuggucaaguuu

SEQ ID NO: 23 - RNA, 21 nt, Artificial Sequence
  Anti-sense strand of siRNA to Target No. 3
acuugaccugggaauguggca

SEQ ID NO: 24 - DNA, 21 bp, Artificial Sequence
  Target No. 4 5′ to 3′
gccacattccctggtcaagtg

SEQ ID NO: 25 - RNA, 21 nt, Artificial Sequence
  Sense strand of siRNA to Target No. 4
cacauucccuggucaaguguu

SEQ ID NO: 26 - RNA, 21 nt, Artificial Sequence
  Anti-sense strand of siRNA to Target No. 4
gacuugaccugggaauguggc

SEQ ID NO: 27 - DNA, 21 bp, Artificial Sequence
  Target No. 5 5′ to 3′
ccacattccctggtcaagtgg

SEQ ID NO: 28 - RNA, 21 nt, Artificial Sequence
  Sense strand of siRNA to Target No. 5
acauucccuggucaagugguu

SEQ ID NO: 29 - RNA, 21 nt, Artificial Sequence
  Anti-sense strand of siRNA to Target No. 5
ccacuugaccugggaaugugg

SEQ ID NO: 30 - DNA, 21 bp, Artificial Sequence
  Target No. 6 5′ to 3′
cacattccctggtcaagtgga

SEQ ID NO: 31 - RNA, 21 nt, Artificial Sequence
  Sense strand of siRNA to Target No. 6
cauucccuggucaaguggauu

SEQ ID NO: 32 - DNA, 21 bp, Artificial Sequence
  Anti-sense strand of siRNA to Target No. 6
tccacuugaccugggaaugug

SEQ ID NO: 33 - DNA, 21 bp, Artificial Sequence
  Target No. 7 5′ to 3′
acattccctggtcaagtggat

SEQ ID NO: 34 - RNA, 21 nt, Artificial Sequence
  Sense strand of siRNA to Target No. 7
auucccuggucaaguggauuu

SEQ ID NO: 35 - DNA, 21 bp, Artificial Sequence
  Anti-sense strand of siRNA to Target No. 7
atccacuugaccugggaaugu

SEQ ID NO: 36 - DNA, 21 bp, Artificial Sequence
  Target No. 8 5′ to 3′
cattccctggtcaagtggatt

SEQ ID NO: 37 - RNA, 21 nt, Artificial Sequence
  Sense strand of siRNA to Target No. 8
uucccuggucaaguggauuuu

SEQ ID NO: 38 - DNA, 21 bp, Artificial Sequence
  Anti-sense strand of siRNA to Target No. 8
aatccacuugaccugggaaug

SEQ ID NO: 39 - DNA, 21 bp, Artificial Sequence
  Target No. 9 5′ to 3′
attccctggtcaagtggattt

SEQ ID NO: 40 - RNA, 21 nt, Artificial Sequence
  Sense strand of siRNA to Target No. 9
ucccuggucaaguggauuuuu

SEQ ID NO: 41 - DNA, 21 bp, Artificial Sequence
  Anti-sense strand of siRNA to Target No. 9
aaatccacuugaccugggaau

SEQ ID NO: 42 - DNA, 21 bp, Artificial Sequence
  Target No. 10 5′ to 3′
ttccctggtcaagtggatttc

SEQ ID NO: 43 - RNA, 21 nt, Artificial Sequence
  Sense strand of siRNA to Target No. 10
cccuggucaaguggauuucuu

SEQ ID NO: 44 - DNA, 21 bp, Artificial Sequence
  Anti-sense strand of siRNA to Target No. 10
gaaatccacuugaccugggaa

SEQ ID NO: 45 - DNA, 21 bp, Artificial Sequence
  Target No. 11 5′ to 3′
tccctggtcaagtggatttcg

SEQ ID NO: 46 - RNA, 21 nt, Artificial Sequence
  Sense strand of siRNA to Target No. 11
ccuggucaaguggauuucguu

SEQ ID NO: 47 - DNA, 21 bp, Artificial Sequence
  Anti-sense strand of siRNA to Target No. 11
cgaaatccacuugaccuggga

SEQ ID NO: 48 - DNA, 21 bp, Artificial Sequence
  Target No. 12 5′ to 3′
ccctggtcaagtggatttcga

SEQ ID NO: 49 - RNA, 21 nt, Artificial Sequence
  Sense strand of siRNA to Target No. 12
cuggucaaguggauuucgauu

SEQ ID NO: 50 - DNA, 21 bp, Artificial Sequence
  Anti-sense strand of siRNA to Target No. 12
ucgaaatccacuugaccuggg

SEQ ID NO: 51 - DNA, 21 bp, Artificial Sequence
  Target No. 13 5′ to 3′
cctggtcaagtggatttcgaa

SEQ ID NO: 52 - RNA, 21 nt, Artificial Sequence
  Sense strand of siRNA to Target No. 13
uggucaaguggauuucgaauu

SEQ ID NO: 53 - DNA, 21 bp, Artificial Sequence
  Anti-sense strand of siRNA to Target No. 13
uucgaaatccacuugaccugg

SEQ ID NO: 54 - DNA, 22 bp, Artificial Sequence
  Target No. 14 5′ to 3′
acattccctggtcaagtggatt

SEQ ID NO: 55 - RNA, 22 nt, Artificial Sequence
  Sense strand of siRNA to Target No. 14
auucccuggucaaguggauuuu

SEQ ID NO: 56 - DNA, 22 bp, Artificial Sequence
  Anti-sense strand of siRNA to Target No. 14
aatccacuugaccugggaaugu

SEQ ID NO: 57 - DNA, 22 bp, Artificial Sequence
  Target No. 15 5′ to 3′
cattccctggtcaagtggattt

SEQ ID NO: 58 - RNA, 22 nt, Artificial Sequence
  Sense strand of siRNA to Target No. 15
uucccuggucaaguggauuuuu

SEQ ID NO: 59 - DNA, 22 bp, Artificial Sequence
  Anti-sense strand of siRNA to Target No. 15
aaatccacuugaccugggaaug

SEQ ID NO: 60 - DNA, 23 bp, Artificial Sequence
  Target No. 16 5′ to 3′
cacattccctggtcaagtggatt

SEQ ID NO: 61 - RNA, 23 nt, Artificial Sequence
  Sense strand of siRNA to Target No. 16
gauucccuggucaaguggauuuu

SEQ ID NO: 62 - DNA, 23 bp, Artificial Sequence
  Anti-sense strand of siRNA to Target No. 16
aatccacuugaccugggaaugug

SEQ ID NO: 63 - DNA, 23 bp, Artificial Sequence
  Target No. 17 5′ to 3′
cattccctggtcaagtggatttc

SEQ ID NO: 64 - RNA, 23 nt, Artificial Sequence
  Sense strand of siRNA to Target No. 17
uucccuggucaaguggauuucuu

SEQ ID NO: 65 - DNA, 23 bp, Artificial Sequence
  Anti-sense strand of siRNA to Target No. 17
gaaatccacuugaccugggaaug

SEQ ID NO: 66 - DNA, 24 bp, Artificial Sequence
  Target No. 18 5′ to 3′
ccacattccctggtcaagtggatt

SEQ ID NO: 67 - RNA, 24 nt, Artificial Sequence
  Sense strand of siRNA to Target No. 18
agauucccuggucaaguggauuuu

SEQ ID NO: 68 - DNA, 24 bp, Artificial Sequence
  Anti-sense strand of siRNA to Target No. 18
aatccacuugaccugggaaugugg

SEQ ID NO: 69 - DNA, 24 bp, Artificial Sequence
  Target No. 19 5′ to 3′
cattccctggtcaagtggatttcg

SEQ ID NO: 70 - RNA, 24 nt, Artificial Sequence
  Sense strand of siRNA to Target No. 19
uucccuggucaaguggauuucguu

SEQ ID NO: 71 - DNA, 24 bp, Artificial Sequence
  Anti-sense strand of siRNA to Target No. 19
cgaaatccacuugaccugggaaug

SEQ ID NO: 72 - DNA, 25 bp, Artificial Sequence
  Target No. 20 5′ to 3′
gccacattccctggtcaagtggatt

SEQ ID NO: 73 - RNA, 25 nt, Artificial Sequence
  Sense strand of siRNA to Target No. 20
cagauucccuggucaaguggauuuu

SEQ ID NO: 74 - DNA, 25 bp, Artificial Sequence
  Anti-sense strand of siRNA to Target No. 20
aatccacuugaccugggaauguggc

SEQ ID NO: 75 - DNA, 25 bp, Artificial Sequence
  Target No. 21 5′ to 3′
cattccctggtcaagtggatttcga

SEQ ID NO: 76 - RNA, 25 nt, Artificial Sequence
  Sense strand of siRNA to Target No. 21
uucccuggucaaguggauuucgauu

SEQ ID NO: 77 - DNA, 25 bp, Artificial Sequence
  Anti-sense strand of siRNA to Target No. 21
ucgaaatccacuugaccugggaaug

SEQ ID NO: 78 - RNA, 21 nt, Artificial Sequence
  sense strand of control siRNA
uucccuggucaaguggauuuu

SEQ ID NO: 79 - RNA, 21 nt, Artificial Sequence
  sense strand of control siRNA
aauccacuugaccagggaaug

SEQ ID NO: 80 - DNA, 21 bp, Artificial Sequence
  sense strand of control siRNA
ccucuacgccaguguguuctt

SEQ ID NO: 81 - DNA, 21 bp, Artificial Sequence
  sense strand of control siRNA
gaacacacuggcguagaggtt

SEQ ID NO: 82 - RNA, 21 nt, Artificial Sequence
  sense strand of control siRNA
aucgucgguagauugccuuuu

SEQ ID NO: 83 - RNA, 21 nt, Artificial Sequence
  sense strand of control siRNA
aaggcaaucuaccgacgauug

SEQ ID NO: 84 - DNA, 21 bp, Artificial Sequence
  sense strand of control siRNA
uucuccgaacgugucacgutt

SEQ ID NO: 85 - DNA, 21 bp, Artificial Sequence
  sense strand of control siRNA
acgugacacguucggagaatt

SEQ ID NO: 86 - DNA, 21 bp, Artificial Sequence
  antisense strand of siRNA
ttuuuuguaccacguccgaag

SEQ ID NO: 87 - DNA, 21 bp, Artificial Sequence
  sense strand of siRNA
ttcuucggacgugguacaaaa

SEQ ID NO: 88 - DNA, 21 bp, Artificial Sequence
  antisense strand of siRNA
ttuuugugugaucgcaugucc

SEQ ID NO: 89 - DNA, 21 bp, Artificial Sequence
  sense strand of siRNA
ttgcacaugcgaucacacaaa

SEQ ID NO: 90 - DNA, 21 bp, Artificial Sequence
  antisense strand of siRNA
ttgaaaccugacagucaacug

SEQ ID NO: 91 - DNA, 21 bp, Artificial Sequence
  sense strand of siRNA
ttcaguugacugucagguuuc

SEQ ID NO: 92 - DNA, 21 bp, Artificial Sequence
  antisense strand of siRNA
ttccucggaaaccugacaguc

SEQ ID NO: 93 - DNA, 21 bp, Artificial Sequence
  sense strand of siRNA
ttgacugucagguuuccgagg

SEQ ID NO: 94 - DNA, 21 bp, Artificial Sequence
  antisense strand of siRNA
ttagucuauuuuugggucggu

SEQ ID NO: 95 - DNA, 21 bp, Artificial Sequence
  sense strand of siRNA
ttaccgacccaaaaauagacu

SEQ ID NO: 96 - DNA, 21 bp, Artificial Sequence
  antisense strand of siRNA
ttgacccuaccguagucacuc

SEQ ID NO: 97 - DNA, 21 bp, Artificial Sequence
  sense strand of siRNA
ttgagugacuacgguaggguc

SEQ ID NO: 98 - RNA, 21 nt, Artificial Sequence
  antisense strand of siRNA
uuuaagucuauuuuugggucg

SEQ ID NO: 99 - RNA, 21 nt, Artificial Sequence
  sense strand of siRNA
accgacccaaaaauagacuua

SEQ ID NO: 100 - RNA, 21 nt, Artificial Sequence
  antisense strand of siRNA
uuguaaaagaaguaggcgcgg

SEQ ID NO: 101 - RNA, 21 nt, Artificial Sequence
  sense strand of siRNA
ccccgcgccuacuucuuuuac

SEQ ID NO: 102 - RNA, 21 nt, Artificial Sequence
  antisense strand of siRNA
uuauaguuuauguggaccacg

SEQ ID NO: 103 - RNA, 21 nt, Artificial Sequence
  sense strand of siRNA
gccgugguccacauaaacuau

SEQ ID NO: 104 - RNA, 21 nt, Artificial Sequence
  antisense strand of siRNA
uuguuuauguggaccacggcu

SEQ ID NO: 105 - RNA, 21 nt, Artificial Sequence
  sense strand of siRNA
ucagccgugguccacauaaac

SEQ ID NO: 106 - RNA, 21 nt, Artificial Sequence
  antisense strand of siRNA
uuuauaguuuaugugggcgcg

SEQ ID NO: 107 - RNA, 21 nt, Artificial Sequence
  sense strand of siRNA
cccgcgcccacauaaacuaua

SEQ ID NO: 108 - RNA, 21 nt, Artificial Sequence
  antisense strand of siRNA
uuuaguuuaugugggcgcggg

SEQ ID NO: 109 - RNA, 21 nt, Artificial Sequence
  sense strand of siRNA
gccccgcgcccacauaaacua

SEQ ID NO: 110 - DNA, 21 bp, Artificial Sequence
  antisense strand of siRNA
ttaagaaguagaccacggcug

SEQ ID NO: 111 - DNA, 21 bp, Artificial Sequence
  sense strand of siRNA
ttcagccguggucuacuucuu

SEQ ID NO: 112 - RNA, 21 nt, Artificial Sequence
  antisense strand of siRNA
uuguaaaagaaguagaccacg

SEQ ID NO: 113 - RNA, 21 nt, Artificial Sequence
  sense strand of siRNA
gccguggucuacuucuuuuac

SEQ ID NO: 114 - DNA, 25 bp, Artificial Sequence
  forward primer 119F
ccacattccctgagttaacatatga

SEQ ID NO: 115 - DNA, 20 bp, Artificial Sequence
  forward primer 114F
ctctgccacattccctggtc

SEQ ID NO: 116 - DNA, 25 bp, Artificial Sequence
  reverse primer 310R
tcttttgataccatcttcagcagaa

SEQ ID NO: 117 - DNA, 30 bp, Artificial Sequence; identical to SEQ ID NO: 6
  probe 232T